SEQUENCE LISTING (170 sequences)

SEQ ID NO: 1 - RNA, 19 nt, synthetic construct
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3: mod_base = OTHER; note = 2'-Fluoro nucleotide
  modified_base 4: mod_base = OTHER; note = 2'O-Methyl nucleotide
  modified_base 5: mod_base = OTHER; note = 2'-Fluoro nucleotide
  modified_base 6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9: mod_base = OTHER; note = 2'-Fluoro nucleotide
  modified_base 10: mod_base = OTHER; note = 2'O-Methyl nucleotide
  modified_base 11: mod_base = OTHER; note = 2'-Fluoro nucleotide
  modified_base 12: mod_base = OTHER; note = 2'O-Methyl nucleotide
  modified_base 13: mod_base = OTHER; note = 2'-Fluoro nucleotide
  modified_base 14: mod_base = OTHER; note = 2'O-Methyl nucleotide
  modified_base 15: mod_base = OTHER; note = 2'-Fluoro nucleotide
  modified_base 16: mod_base = OTHER; note = 2'O-Methyl nucleotide
  modified_base 17: mod_base = OTHER; note = 2'-Fluoro nucleotide
  modified_base 18: mod_base = OTHER; note = 2'O-Methyl nucleotide
  modified_base 19: mod_base = OTHER; note = 2'-Fluoro nucleotide
  source 1..19: mol_type = other RNA; note = siRNA1 sense strand
ctaccagcagtcgatgaaa

SEQ ID NO: 2 - RNA, 19 nt, synthetic construct
  modified_base 1: mod_base = OTHER; note = 2'O-Methyl nucleotide phosphorothioate
  modified_base 2: mod_base = OTHER; note = 2'O-Methyl nucleotide phosphorothioate
  modified_base 3: mod_base = OTHER; note = 2'-Fluoro nucleotide
  modified_base 4: mod_base = OTHER; note = 2'O-Methyl nucleotide
  modified_base 5: mod_base = OTHER; note = 2'-Fluoro nucleotide
  modified_base 6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9: mod_base = OTHER; note = 2'-Fluoro nucleotide
  modified_base 10: mod_base = OTHER; note = 2'O-Methyl nucleotide
  modified_base 11: mod_base = OTHER; note = 2'-Fluoro nucleotide
  modified_base 12: mod_base = OTHER; note = 2'O-Methyl nucleotide
  modified_base 13: mod_base = OTHER; note = 2'-Fluoro nucleotide
  modified_base 14: mod_base = OTHER; note = 2'O-Methyl nucleotide
  modified_base 15: mod_base = OTHER; note = 2'-Fluoro nucleotide
  modified_base 16: mod_base = OTHER; note = 2'O-Methyl nucleotide
  modified_base 17: mod_base = OTHER; note = 2'-Fluoro nucleotide
  modified_base 18: mod_base = OTHER; note = 2'O-Methyl nucleotide
  modified_base 19: mod_base = OTHER; note = 2'-Fluoro nucleotide
  source 1..19: mol_type = other RNA; note = siRNA2 sense strand
gaaacgggccatttcataa

SEQ ID NO: 3 - RNA, 19 nt, synthetic construct
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3: mod_base = OTHER; note = 2'-Fluoro nucleotide
  modified_base 4: mod_base = OTHER; note = 2'O-Methyl nucleotide
  modified_base 5: mod_base = OTHER; note = 2'-Fluoro nucleotide
  modified_base 6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9: mod_base = OTHER; note = 2'-Fluoro nucleotide
  modified_base 10: mod_base = OTHER; note = 2'O-Methyl nucleotide
  modified_base 11: mod_base = OTHER; note = 2'-Fluoro nucleotide
  modified_base 12: mod_base = OTHER; note = 2'O-Methyl nucleotide
  modified_base 13: mod_base = OTHER; note = 2'-Fluoro nucleotide
  modified_base 14: mod_base = OTHER; note = 2'O-Methyl nucleotide
  modified_base 15: mod_base = OTHER; note = 2'-Fluoro nucleotide
  modified_base 16: mod_base = OTHER; note = 2'O-Methyl nucleotide
  modified_base 17: mod_base = OTHER; note = 2'-Fluoro nucleotide
  modified_base 18: mod_base = OTHER; note = 2'O-Methyl nucleotide
  modified_base 19: mod_base = OTHER; note = 2'-Fluoro nucleotide
  source 1..19: mol_type = other RNA; note = AHSA1 siRNA sense strand
tctcgtggccttaatgaaa

SEQ ID NO: 4 - protein, 220 aa, Homo sapiens
MSMGLEITGTALAVLGWLGTIVCCALPMWRVSAFIGSNIITSQNIWEGLWMNCVVQSTGQMQCKVYDSLLALPQDLQAARALIVVAILLAAFGLLVALVGAQCTNCVQDDTAKAKITIVAGVLFLLAALLTLVPVSWSANTIIRDFYNPVVPEAQKREMGAGLYVGWAAAALQLLGGALLCCSCPPREKKYTATKVVYSAPRSTGPGASLGTGYDRKDYV

SEQ ID NO: 5 - RNA, 21 nt, synthetic construct
  modified_base 1: mod_base = OTHER; note = 2'-Fluoro; phosphorothioate
  modified_base 2: mod_base = OTHER; note = 2'-Fluoro; phosphorothioate
  modified_base 3: mod_base = OTHER; note = 2'O-Methyl nucleotide
  modified_base 4: mod_base = OTHER; note = 2'-Fluoro nucleotide
  modified_base 5: mod_base = OTHER; note = 2'O-Methyl nucleotide
  modified_base 6: mod_base = OTHER; note = 2'-Fluoro nucleotide
  modified_base 7: mod_base = OTHER; note = 2'O-Methyl nucleotide
  modified_base 8: mod_base = OTHER; note = 2'-Fluoro nucleotide
  modified_base 9: mod_base = OTHER; note = 2'O-Methyl nucleotide
  modified_base 10: mod_base = OTHER; note = 2'-Fluoro nucleotide
  modified_base 11: mod_base = OTHER; note = 2'O-Methyl nucleotide
  modified_base 12: mod_base = OTHER; note = 2'-Fluoro nucleotide
  modified_base 13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17: mod_base = OTHER; note = 2'O-Methyl nucleotide
  modified_base 18: mod_base = OTHER; note = 2'-Fluoro nucleotide
  modified_base 19: mod_base = OTHER; note = 2'O-methyl nucleotide phosphorothioate
  modified_base 20: mod_base = OTHER; note = 2'O-methyl nucleotide phosphorothioate
  modified_base 21: mod_base = OTHER; note = 2'O-Methyl nucleotide
  source 1..21: mol_type = other RNA; note = siRNA1 anti-sense strand
tttcatcgactgctggtagtt

SEQ ID NO: 6 - RNA, 21 nt, synthetic construct
  modified_base 1: mod_base = OTHER; note = 2'-Fluoro; phosphorothioate
  modified_base 2: mod_base = OTHER; note = 2'-Fluoro; phosphorothioate
  modified_base 3: mod_base = OTHER; note = 2'O-Methyl nucleotide
  modified_base 4: mod_base = OTHER; note = 2'-Fluoro nucleotide
  modified_base 5: mod_base = OTHER; note = 2'O-Methyl nucleotide
  modified_base 6: mod_base = OTHER; note = 2'-Fluoro nucleotide
  modified_base 7: mod_base = OTHER; note = 2'O-Methyl nucleotide
  modified_base 8: mod_base = OTHER; note = 2'-Fluoro nucleotide
  modified_base 9: mod_base = OTHER; note = 2'O-Methyl nucleotide
  modified_base 10: mod_base = OTHER; note = 2'-Fluoro nucleotide
  modified_base 11: mod_base = OTHER; note = 2'O-Methyl nucleotide
  modified_base 12: mod_base = OTHER; note = 2'-Fluoro nucleotide
  modified_base 13: mod_base = OTHER; note = 2'-Fluoro nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17: mod_base = OTHER; note = 2'O-Methyl nucleotide
  modified_base 18: mod_base = OTHER; note = 2'-Fluoro nucleotide
  modified_base 19: mod_base = OTHER; note = 2'O-methyl nucleotide phosphorothioate
  modified_base 20: mod_base = OTHER; note = 2'O-methyl nucleotide phosphorothioate
  modified_base 21: mod_base = OTHER; note = 2'O-Methyl nucleotide
  source 1..21: mol_type = other RNA; note = siRNA2 anti-sense strand
ttatgaaatggcccgtttctt

SEQ ID NO: 7 - DNA, 20 bp, synthetic construct
  source 1..20: mol_type = other DNA; note = Primer mEef1a1_F
cgttctttttcgcaacgggt

SEQ ID NO: 8 - DNA, 20 bp, synthetic construct
  source 1..20: mol_type = other DNA; note = Primer mEef1a1_R
ttgccggaatctacgtgtcc

SEQ ID NO: 9 - DNA, 20 bp, synthetic construct
  source 1..20: mol_type = other DNA; note = Primer mCldn3_F
gcacccaccaagatcctcta

SEQ ID NO: 10 - DNA, 20 bp, synthetic construct
  source 1..20: mol_type = other DNA; note = Primer mCldn3_R
tcgtctgtcaccatctggaa

SEQ ID NO: 11 - DNA, 26 bp, synthetic construct
  source 1..26: mol_type = other DNA; note = Primer mCyp7a1_F
agcaactaaacaacctgccagtacta

SEQ ID NO: 12 - DNA, 21 bp, synthetic construct
  source 1..21: mol_type = other DNA; note = Primer mCyp7a1_R
gtccggatattcaaggatgca

SEQ ID NO: 13 - DNA, 20 bp, synthetic construct
  source 1..20: mol_type = other DNA; note = Primer mCyp7b1_F
aattggacagcttggtctgc

SEQ ID NO: 14 - DNA, 20 bp, synthetic construct
  source 1..20: mol_type = other DNA; note = Primer mCyp7b1_R
ttctcggatgatgctggagt

SEQ ID NO: 15 - DNA, 20 bp, synthetic construct
  source 1..20: mol_type = other DNA; note = Primer mCyp27a1_F
gtggacaacctcctttggga

SEQ ID NO: 16 - DNA, 21 bp, synthetic construct
  source 1..21: mol_type = other DNA; note = Primer mCyp27a1_R
ttgctctccttgtgcgatgaa

SEQ ID NO: 17 - DNA, 20 bp, synthetic construct
  source 1..20: mol_type = other DNA; note = Primer mGsta1_F
ggctttcaagattcagtgaa

SEQ ID NO: 18 - DNA, 21 bp, synthetic construct
  source 1..21: mol_type = other DNA; note = Primer mGsta1_R
tagccaggatcaacaattgct

SEQ ID NO: 19 - DNA, 22 bp, synthetic construct
  source 1..22: mol_type = other DNA; note = Primer mCyp4a14_F
cccaaaggtatcacagccacaa

SEQ ID NO: 20 - DNA, 21 bp, synthetic construct
  source 1..21: mol_type = other DNA; note = Primer mCyp4a14_R
cagcaattcaaagcggagcag

SEQ ID NO: 21 - DNA, 21 bp, synthetic construct
  source 1..21: mol_type = other DNA; note = Primer mOst1b_F
agatgcggctccttggaatta

SEQ ID NO: 22 - DNA, 22 bp, synthetic construct
  source 1..22: mol_type = other DNA; note = Primer mOst1b_R
tggctgcttctttcgatttctg

SEQ ID NO: 23 - DNA, 24 bp, synthetic construct
  source 1..24: mol_type = other DNA; note = Primer mOatp1a1_F
gccaacgcaagatccaacagagtg

SEQ ID NO: 24 - DNA, 22 bp, synthetic construct
  source 1..22: mol_type = other DNA; note = Primer mOatp1a1_R
tcgggccaacaatcttccccat

SEQ ID NO: 25 - DNA, 22 bp, synthetic construct
  source 1..22: mol_type = other DNA; note = Primer mOatp1b2_F
tggaaggcatagggtaggcggt

SEQ ID NO: 26 - DNA, 22 bp, synthetic construct
  source 1..22: mol_type = other DNA; note = Primer mOatp1b2_R
tgggcagctttgcttggatgct

SEQ ID NO: 27 - DNA, 21 bp, synthetic construct
  source 1..21: mol_type = other DNA; note = Primer mNtcp_F
aatccaagctgcagacgcacc

SEQ ID NO: 28 - DNA, 23 bp, synthetic construct
  source 1..23: mol_type = other DNA; note = Primer mNtcp_R
gcatcttctgttgcagcagcctt

SEQ ID NO: 29 - DNA, 21 bp, synthetic construct
  source 1..21: mol_type = other DNA; note = Primer mMDR2_F
tggccgatgtgtgtgagtaca

SEQ ID NO: 30 - DNA, 18 bp, synthetic construct
  source 1..18: mol_type = other DNA; note = Primer mMDR2_R
tgcctggcaccaaaaggt

SEQ ID NO: 31 - DNA, 20 bp, synthetic construct
  source 1..20: mol_type = other DNA; note = Primer mMrp3_F
gggctgccttgccctgctac

SEQ ID NO: 32 - DNA, 20 bp, synthetic construct
  source 1..20: mol_type = other DNA; note = Primer mMrp3_R
ccgagggccgtcttgagcct

SEQ ID NO: 33 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
caacatcatcacgtcgcagaa

SEQ ID NO: 34 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
accaactgcgtgcaggacgaa

SEQ ID NO: 35 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
ggccaacaccattatccggga

SEQ ID NO: 36 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
cgcagaagcgcgagatgggca

SEQ ID NO: 37 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
cagaagcgcgagatgggcgca

SEQ ID NO: 38 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
cgcgagaagaagtacacggca

SEQ ID NO: 39 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
accaaggtcgtctactccgca

SEQ ID NO: 40 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
aaggtcgtctactccgcgcca

SEQ ID NO: 41 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
aggtcgtctactccgcgccga

SEQ ID NO: 42 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
ggtcgtctactccgcgccgca

SEQ ID NO: 43 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
ctgggcacaggctacgaccga

SEQ ID NO: 44 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
tgggcacaggctacgaccgca

SEQ ID NO: 45 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
ggcacaggctacgaccgcaaa

SEQ ID NO: 46 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
gcacaggctacgaccgcaaga

SEQ ID NO: 47 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
ctacgaccgcaaggactacga

SEQ ID NO: 48 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
acgaccgcaaggactacgtca

SEQ ID NO: 49 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
cgaccgcaaggactacgtcta

SEQ ID NO: 50 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
ccgcaaggactacgtctaaga

SEQ ID NO: 51 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
ctacgtctaagggacagacga

SEQ ID NO: 52 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
acgtctaagggacagacgcaa

SEQ ID NO: 53 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
cgtctaagggacagacgcaga

SEQ ID NO: 54 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
ctggagcgcgcaccaggccaa

SEQ ID NO: 55 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
ttgcgggccgggcagtcgaca

SEQ ID NO: 56 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
gccgggcagtcgacttcggga

SEQ ID NO: 57 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
ccagggaccaacctgcatgga

SEQ ID NO: 58 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
atggactgtgaaacctcacca

SEQ ID NO: 59 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
gtgaccgccaatacttgacca

SEQ ID NO: 60 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
gaccgccaatacttgaccaca

SEQ ID NO: 61 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
ccaatacttgaccaccccgta

SEQ ID NO: 62 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
aatacttgaccaccccgtcga

SEQ ID NO: 63 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
atacttgaccaccccgtcgaa

SEQ ID NO: 64 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
acttgaccaccccgtcgagca

SEQ ID NO: 65 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
accccgtcgagccccatcgga

SEQ ID NO: 66 - RNA, 21 nt, synthetic construct
  source 1..21: mol_type = other RNA
  modified_base 1: mod_base = OTHER; note = 2'O-methyl nucleotide phosphorothioate
  modified_base 2: mod_base = OTHER; note = 2'O-methyl nucleotide phosphorothioate
  modified_base 3..6: mod_base = OTHER; note = 2'O-Methyl nucleotide
  modified_base 7: mod_base = OTHER; note = 2'-Fluoro nucleotide
  modified_base 8: mod_base = OTHER; note = 2'O-Methyl nucleotide
  modified_base 9..11: mod_base = OTHER; note = 2'-Fluoro nucleotide
  modified_base 12..21: mod_base = OTHER; note = 2'O-Methyl nucleotide
cccgtcgagccccatcgggca

SEQ ID NO: 67 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
ttctgcgacgtgatgatgttgct

SEQ ID NO: 68 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
ttcgtcctgcacgcagttggtgc

SEQ ID NO: 69 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
tcccggataatggtgttggccga

SEQ ID NO: 70 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
tgcccatctcgcgcttctgcgcc

SEQ ID NO: 71 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
tgcgcccatctcgcgcttctgcg

SEQ ID NO: 72 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
tgccgtgtacttcttctcgcgtg

SEQ ID NO: 73 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
tgcggagtagacgaccttggtgg

SEQ ID NO: 74 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
tggcgcggagtagacgaccttgg

SEQ ID NO: 75 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
tcggcgcggagtagacgaccttg

SEQ ID NO: 76 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
tgcggcgcggagtagacgacctt

SEQ ID NO: 77 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
tcggtcgtagcctgtgcccaggc

SEQ ID NO: 78 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
tgcggtcgtagcctgtgcccagg

SEQ ID NO: 79 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
tttgcggtcgtagcctgtgccca

SEQ ID NO: 80 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
tcttgcggtcgtagcctgtgccc

SEQ ID NO: 81 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
tcgtagtccttgcggtcgtagcc

SEQ ID NO: 82 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
tgacgtagtccttgcggtcgtag

SEQ ID NO: 83 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
tagacgtagtccttgcggtcgta

SEQ ID NO: 84 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
tcttagacgtagtccttgcggtc

SEQ ID NO: 85 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
tcgtctgtcccttagacgtagtc

SEQ ID NO: 86 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
ttgcgtctgtcccttagacgtag

SEQ ID NO: 87 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
tctgcgtctgtcccttagacgta

SEQ ID NO: 88 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
ttggcctggtgcgcgctccagct

SEQ ID NO: 89 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
tgtcgactgcccggcccgcaaag

SEQ ID NO: 90 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
tcccgaagtcgactgcccggccc

SEQ ID NO: 91 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
tccatgcaggttggtccctgggc

SEQ ID NO: 92 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
tggtgaggtttcacagtccatgc

SEQ ID NO: 93 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
tggtcaagtattggcggtcaccc

SEQ ID NO: 94 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
tgtggtcaagtattggcggtcac

SEQ ID NO: 95 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
tacggggtggtcaagtattggcg

SEQ ID NO: 96 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
tcgacggggtggtcaagtattgg

SEQ ID NO: 97 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
ttcgacggggtggtcaagtattg

SEQ ID NO: 98 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
tgctcgacggggtggtcaagtat

SEQ ID NO: 99 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
tccgatggggctcgacggggtgg

SEQ ID NO: 100 - RNA, 23 nt, synthetic construct
  source 1..23: mol_type = other RNA
  modified_base 1: mod_base = OTHER; note = 2'O-methyl nucleotide phosphorothioate
  modified_base 2: mod_base = OTHER; note = 2'-Fluoro nucleotide phosphorothioate
  modified_base 3..5: mod_base = OTHER; note = 2'O-Methyl nucleotide
  modified_base 6: mod_base = OTHER; note = 2'-Fluoro nucleotide
  modified_base 7..13: mod_base = OTHER; note = 2'O-Methyl nucleotide
  modified_base 14: mod_base = OTHER; note = 2'-Fluoro nucleotide
  modified_base 15: mod_base = OTHER; note = 2'O-Methyl nucleotide
  modified_base 16: mod_base = OTHER; note = 2'-Fluoro nucleotide
  modified_base 17..20: mod_base = OTHER; note = 2'O-methyl nucleotide
  modified_base 21..22: mod_base = OTHER; note = 2'O-methyl nucleotide phosphorothioate
  modified_base 23: mod_base = OTHER; note = 2'O-methyl nucleotide
tgcccgatggggctcgacggggt

SEQ ID NO: 101 - RNA, 21 nt, synthetic construct
  source 1..21: mol_type = other RNA
caacatcatcacgtcgcagaa

SEQ ID NO: 102 - RNA, 21 nt, synthetic construct
  source 1..21: mol_type = other RNA
accaactgcgtgcaggacgaa

SEQ ID NO: 103 - RNA, 21 nt, synthetic construct
  source 1..21: mol_type = other RNA
ggccaacaccattatccggga

SEQ ID NO: 104 - RNA, 21 nt, synthetic construct
  source 1..21: mol_type = other RNA
cgcagaagcgcgagatgggca

SEQ ID NO: 105 - RNA, 21 nt, synthetic construct
  source 1..21: mol_type = other RNA
cagaagcgcgagatgggcgca

SEQ ID NO: 106 - RNA, 21 nt, synthetic construct
  source 1..21: mol_type = other RNA
cgcgagaagaagtacacggca

SEQ ID NO: 107 - RNA, 21 nt, synthetic construct
  source 1..21: mol_type = other RNA
accaaggtcgtctactccgca

SEQ ID NO: 108 - RNA, 21 nt, synthetic construct
  source 1..21: mol_type = other RNA
aaggtcgtctactccgcgcca

SEQ ID NO: 109 - RNA, 21 nt, synthetic construct
  source 1..21: mol_type = other RNA
aggtcgtctactccgcgccga

SEQ ID NO: 110 - RNA, 21 nt, synthetic construct
  source 1..21: mol_type = other RNA
ggtcgtctactccgcgccgca

SEQ ID NO: 111 - RNA, 21 nt, synthetic construct
  source 1..21: mol_type = other RNA
ctgggcacaggctacgaccga

SEQ ID NO: 112 - RNA, 21 nt, synthetic construct
  source 1..21: mol_type = other RNA
tgggcacaggctacgaccgca

SEQ ID NO: 113 - RNA, 21 nt, synthetic construct
  source 1..21: mol_type = other RNA
ggcacaggctacgaccgcaaa

SEQ ID NO: 114 - RNA, 21 nt, synthetic construct
  source 1..21: mol_type = other RNA
gcacaggctacgaccgcaaga

SEQ ID NO: 115 - RNA, 21 nt, synthetic construct
  source 1..21: mol_type = other RNA
ctacgaccgcaaggactacga

SEQ ID NO: 116 - RNA, 21 nt, synthetic construct
  source 1..21: mol_type = other RNA
acgaccgcaaggactacgtca

SEQ ID NO: 117 - RNA, 21 nt, synthetic construct
  source 1..21: mol_type = other RNA
cgaccgcaaggactacgtcta

SEQ ID NO: 118 - RNA, 21 nt, synthetic construct
  source 1..21: mol_type = other RNA
ccgcaaggactacgtctaaga

SEQ ID NO: 119 - RNA, 21 nt, synthetic construct
  source 1..21: mol_type = other RNA
ctacgtctaagggacagacga

SEQ ID NO: 120 - RNA, 21 nt, synthetic construct
  source 1..21: mol_type = other RNA
acgtctaagggacagacgcaa

SEQ ID NO: 121 - RNA, 21 nt, synthetic construct
  source 1..21: mol_type = other RNA
cgtctaagggacagacgcaga

SEQ ID NO: 122 - RNA, 21 nt, synthetic construct
  source 1..21: mol_type = other RNA
ctggagcgcgcaccaggccaa

SEQ ID NO: 123 - RNA, 21 nt, synthetic construct
  source 1..21: mol_type = other RNA
ttgcgggccgggcagtcgaca

SEQ ID NO: 124 - RNA, 21 nt, synthetic construct
  source 1..21: mol_type = other RNA
gccgggcagtcgacttcggga

SEQ ID NO: 125 - RNA, 21 nt, synthetic construct
  source 1..21: mol_type = other RNA
ccagggaccaacctgcatgga

SEQ ID NO: 126 - RNA, 21 nt, synthetic construct
  source 1..21: mol_type = other RNA
atggactgtgaaacctcacca

SEQ ID NO: 127 - RNA, 21 nt, synthetic construct
  source 1..21: mol_type = other RNA
gtgaccgccaatacttgacca

SEQ ID NO: 128 - RNA, 21 nt, synthetic construct
  source 1..21: mol_type = other RNA
gaccgccaatacttgaccaca

SEQ ID NO: 129 - RNA, 21 nt, synthetic construct
  source 1..21: mol_type = other RNA
ccaatacttgaccaccccgta

SEQ ID NO: 130 - RNA, 21 nt, synthetic construct
  source 1..21: mol_type = other RNA
aatacttgaccaccccgtcga

SEQ ID NO: 131 - RNA, 21 nt, synthetic construct
  source 1..21: mol_type = other RNA
atacttgaccaccccgtcgaa

SEQ ID NO: 132 - RNA, 21 nt, synthetic construct
  source 1..21: mol_type = other RNA
acttgaccaccccgtcgagca

SEQ ID NO: 133 - RNA, 21 nt, synthetic construct
  source 1..21: mol_type = other RNA
accccgtcgagccccatcgga

SEQ ID NO: 134 - RNA, 21 nt, synthetic construct
  source 1..21: mol_type = other RNA
cccgtcgagccccatcgggca

SEQ ID NO: 135 - RNA, 23 nt, synthetic construct
  source 1..23: mol_type = other RNA
ttctgcgacgtgatgatgttgct

SEQ ID NO: 136 - RNA, 23 nt, synthetic construct
  source 1..23: mol_type = other RNA
ttcgtcctgcacgcagttggtgc

SEQ ID NO: 137 - RNA, 23 nt, synthetic construct
  source 1..23: mol_type = other RNA
tcccggataatggtgttggccga

SEQ ID NO: 138 - RNA, 23 nt, synthetic construct
  source 1..23: mol_type = other RNA
tgcccatctcgcgcttctgcgcc

SEQ ID NO: 139 - RNA, 23 nt, synthetic construct
  source 1..23: mol_type = other RNA
tgcgcccatctcgcgcttctgcg

SEQ ID NO: 140 - RNA, 23 nt, synthetic construct
  source 1..23: mol_type = other RNA
tgccgtgtacttcttctcgcgtg

SEQ ID NO: 141 - RNA, 23 nt, synthetic construct
  source 1..23: mol_type = other RNA
tgcggagtagacgaccttggtgg

SEQ ID NO: 142 - RNA, 23 nt, synthetic construct
  source 1..23: mol_type = other RNA
tggcgcggagtagacgaccttgg

SEQ ID NO: 143 - RNA, 23 nt, synthetic construct
  source 1..23: mol_type = other RNA
tcggcgcggagtagacgaccttg

SEQ ID NO: 144 - RNA, 23 nt, synthetic construct
  source 1..23: mol_type = other RNA
tgcggcgcggagtagacgacctt

SEQ ID NO: 145 - RNA, 23 nt, synthetic construct
  source 1..23: mol_type = other RNA
tcggtcgtagcctgtgcccaggc

SEQ ID NO: 146 - RNA, 23 nt, synthetic construct
  source 1..23: mol_type = other RNA
tgcggtcgtagcctgtgcccagg

SEQ ID NO: 147 - RNA, 23 nt, synthetic construct
  source 1..23: mol_type = other RNA
tttgcggtcgtagcctgtgccca

SEQ ID NO: 148 - RNA, 23 nt, synthetic construct
  source 1..23: mol_type = other RNA
tcttgcggtcgtagcctgtgccc

SEQ ID NO: 149 - RNA, 23 nt, synthetic construct
  source 1..23: mol_type = other RNA
tcgtagtccttgcggtcgtagcc

SEQ ID NO: 150 - RNA, 23 nt, synthetic construct
  source 1..23: mol_type = other RNA
tgacgtagtccttgcggtcgtag

SEQ ID NO: 151 - RNA, 23 nt, synthetic construct
  source 1..23: mol_type = other RNA
tagacgtagtccttgcggtcgta

SEQ ID NO: 152 - RNA, 23 nt, synthetic construct
  source 1..23: mol_type = other RNA
tcttagacgtagtccttgcggtc

SEQ ID NO: 153 - RNA, 23 nt, synthetic construct
  source 1..23: mol_type = other RNA
tcgtctgtcccttagacgtagtc

SEQ ID NO: 154 - RNA, 23 nt, synthetic construct
  source 1..23: mol_type = other RNA
ttgcgtctgtcccttagacgtag

SEQ ID NO: 155 - RNA, 23 nt, synthetic construct
  source 1..23: mol_type = other RNA
tctgcgtctgtcccttagacgta

SEQ ID NO: 156 - RNA, 23 nt, synthetic construct
  source 1..23: mol_type = other RNA
ttggcctggtgcgcgctccagct

SEQ ID NO: 157 - RNA, 23 nt, synthetic construct
  source 1..23: mol_type = other RNA
tgtcgactgcccggcccgcaaag

SEQ ID NO: 158 - RNA, 23 nt, synthetic construct
  source 1..23: mol_type = other RNA
tcccgaagtcgactgcccggccc

SEQ ID NO: 159 - RNA, 23 nt, synthetic construct
  source 1..23: mol_type = other RNA
tccatgcaggttggtccctgggc

SEQ ID NO: 160 - RNA, 23 nt, synthetic construct
  source 1..23: mol_type = other RNA
tggtgaggtttcacagtccatgc

SEQ ID NO: 161 - RNA, 23 nt, synthetic construct
  source 1..23: mol_type = other RNA
tggtcaagtattggcggtcaccc

SEQ ID NO: 162 - RNA, 23 nt, synthetic construct
  source 1..23: mol_type = other RNA
tgtggtcaagtattggcggtcac

SEQ ID NO: 163 - RNA, 23 nt, synthetic construct
  source 1..23: mol_type = other RNA
tacggggtggtcaagtattggcg

SEQ ID NO: 164 - RNA, 23 nt, synthetic construct
  source 1..23: mol_type = other RNA
tcgacggggtggtcaagtattgg

SEQ ID NO: 165 - RNA, 23 nt, synthetic construct
  source 1..23: mol_type = other RNA
ttcgacggggtggtcaagtattg

SEQ ID NO: 166 - RNA, 23 nt, synthetic construct
  source 1..23: mol_type = other RNA
tgctcgacggggtggtcaagtat

SEQ ID NO: 167 - RNA, 23 nt, synthetic construct
  source 1..23: mol_type = other RNA
tccgatggggctcgacggggtgg

SEQ ID NO: 168 - RNA, 23 nt, synthetic construct
  source 1..23: mol_type = other RNA
tgcccgatggggctcgacggggt

SEQ ID NO: 169 - protein, 220 aa, Homo sapiens
DIVMSQSPSSLAVSVGEKVTMSCKSSPSLLYSNNQKNYLAWYQQKPGQSPKLLIYWASTRESGVPDRFTGSGSGTDFTLTISSVKAEDLAVYYCQQYYSFPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 170 - protein, 451 aa, Homo sapiens
EVQLQQSGPELVKPGGSMKISCKASGYSFTGYTMNWVKQSHGKNLEWIGLINPYNDGTNYNQKFKGKATLTVDKSSSTAYMELLSLTSEDSAVYYCSKKGGGYGGTWFAYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

(19) United States
(12) Patent Application Publication
(10) Pub. No.: US 2025/0382622 A1
(43) Pub. Date: Dec. 18, 2025

(54) METHODS FOR PREVENTION, DELAY OF PROGRESSION OR TREATMENT OF CHOLESTASIS AND/OR FIBROSIS ASSOCIATED WITH CHOLESTASIS

(71) Applicant: UNIVERSITÄT BERN, Bern (CH)

(72) Inventors: Felix BAIER, Ostermundigen (CH); Deborah KEOGH-STROKA, Arni (CH); Daniel CANDINAS VON ALBERTINI, Bern (CH); Nicolas MELIN, Saint Louis (FR)

(21) Appl. No.: 18/877,523

(22) PCT Filed: Jun. 28, 2023
(86) PCT No.: PCT/EP2023/067574

§ 371 Date: Dec. 20, 2024

(30) Foreign Application Priority Data
Jun. 30, 2022 (EP) 22182112.7

(51) Int. Cl. C12N 15/113; A61K 47/54; A61P 1/16; C07K 16/28

(52) U.S. Cl. CPC C12N 15/1138; A61K 47/549; A61P 1/16; C07K 16/28

(57) ABSTRACT

The present invention relates to a method of prevention, delay of progression or treatment of cholestasis and/or fibrosis associated with cholestasis.

### FIELD OF THE INVENTION

The present invention relates to a method of prevention, delay of progression or treatment of cholestasis and/or fibrosis associated with cholestasis.

### BACKGROUND OF THE INVENTION

Cholestasis is characterized by a reduction or stagnation of bile flow in the liver. This can be caused either by obstruction of extrahepatic bile ducts, or by intrahepatic defects in bile synthesis or circulation. The aetiology includes drug intoxications, alcoholic or viral hepatitis, biliary atresia, gallstones and genetic diseases. Cholestatic liver diseases are a major personal and economic burden for patients and society; liver dysfunction due to cholestasis accounts for about 10% of all liver transplantations that are performed in Europe¹. Responsible for this intriguingly high number is the distressing absence of effective treatment options for cholestatic diseases like primary sclerosing cholangitis (PSC) or primary biliary cirrhosis (PBC). The current first line of treatment involves the administration of the immunomodulatory- and bicarbonate secretion stimulating drug ursodeoxycholic acid (UDCA), which improves transplantation free survival in about 60% of PBC patients and shows only limited efficacy on PSC patients². New therapeutic approaches are focused on immunomodulatory strategies³⁻⁵, microbiota alterations^{6,7} or activation of FGF19/FXR signalling for anti-fibrotic effects and improvements of bile acid export and detoxification⁸⁻¹⁰. Several groups therefore suggested PPAR agonists for the treatment of cholestasis¹¹⁻¹³ to increase the efflux of toxic bile acids. At present, the treatment of chronic cholestatic diseases is difficult and often ineffective. New treatments regarding cholestasis and/or fibrosis associated with cholestasischolestasis and/or fibrosis associated with cholestasis are necessary in order to meet the high medical need.

### SUMMARY OF THE INVENTION

It has now unexpectedly been found by the inventors of the present application that Claudin-3 inhibition is a possible therapeutic approach for treating cholestasis and/or fibrosis associated with cholestasis. Using siRNA targeting Claudin-3 promising amelioration of cholestatic liver injury could be achieved. Taking these unexpected findings into account, the inventors herewith provide the present invention in its following aspects.

In a first aspect, the present invention provides an agent which inhibits the expression and/or activity of Claudin-3 for use in a method of prevention, delay of progression or treatment of cholestasis and/or fibrosis associated with cholestasis.

In a second aspect, the present invention provides a composition comprising an agent which inhibits the expression and/or activity of Claudin-3 and a pharmaceutically acceptable carrier for use in a method of prevention, delay of progression or treatment of cholestasis and/or fibrosis associated with cholestasis.

In a third aspect, the present invention provides a dosage form for the prevention, delay of progression or treatment of cholestasis and/or fibrosis associated with cholestasis, comprising an agent which inhibits the expression and/or activity of Claudin-3 or a composition comprising said agent, and a pharmaceutically acceptable carrier.

In a fourth aspect, the present invention provides a siRNA targeting Claudin-3.

### BRIEF DESCRIPTION OF THE FIGURES

FIG. 1. Claudin-3 knockout leads to impaired bile acid metabolism, diluted bile and impaired uptake of the fluorescent tracer FITC Dextran. (A) Metascape analysis. (B) Differential gene expression analysis comparing native Cldn3^{+/+} and Cldn3^{-/-} liver tissue (n=3,) (C) RT-qPCR for genes involved in bile acid synthesis (n=4-6, students t-test). (D) Quantification of total bile acid levels in gallbladder bile (n=3, students t-test). (E) Quantification of bile autofluorescence (excitation 489 nm/emission 534 nm, n=4/5, students t-test). (F) Schematic drawing showing the setup to quantify bile flow and FITC Dextran (40 kDa) blood biliary passage over time (IV, intra-vital) (Scheme contains modified vector images from smart.servier.com, under Creative Commons Attribution 3.0 unported license) (G) Quantification of bile flow as an average over a period of 20 min (n=4, students t-test). (H) Quantification of intravitally injected FITC Dextran (40 kDa) blood-biliary-passage over time (0-30 minutes; n=3-5 per timepoint, 33-40 minutes; n=1 students t-test, *P<0.05, **P<0.01, ns; non-significant). (I) Picture of mice injected with FITC Dextran (40 kDa), 20 minutes post IV injection. Fluorescent FITC Dextran (40 kDa) signal (white) was less prominent in Cldn3^{-/-} livers. Timeseries images acquired with IVIS® Spectrum system (n=1). (J) Quantification of the FITC Dextran (40 kDa) signal in the timeseries.
(K) Orthoslice images from z-stacks of mouse livers (25 um thick), 20 minutes post IV injection of FITC Dextran (40 kDa, FITC signal in light grey). (L) Quantification of FITC Dextran signal in z-stacks of the livers from (K) (n=3-4, students t-test). (FD-40; FITC Dextran (40 kDa), RFU; relative fluorescence unit)

FIG. 2. Absence of claudin-3 protects from obstructive cholestatic liver injury. (A) Photographs of livers pre- and post-bile duct ligation (B) Haematoxylin and eosin stained liver tissue pre- and post-bile duct ligation. Encircled areas highlight necrotic areas. (C) Quantification of tissue necrosis (n=5 for control; 2 days; 7 days, n=3/4 for 12 days, students t-test, **P<0.01). (D) Serum ALT levels. (E) Serum AST levels. (F) Serum ALP levels. (serum analysis: n=5 for control, 2 days, 7 days and n=3-4 for 12 days, students t-test). (G) Bacterial translocation quantified as colony forming units (CFU) per gram liver tissue (n=9-7 for 2 days and n=4 for 7 days, students t-test, +/+=Cldn3^{+/+}, −/−=Cldn3^{-/-}). (H) FACS analysis of immune cell populations at 7 days post bile duct ligation (n=3-4, students t-test).

FIG. 3. Reduced bile acid concentration in liver and gallbladder bile following common bile duct ligation (BDL) (A) Photographs of bile collected from the gallbladder 7 days following BDL (representative image from n=5). (B) TBA quantification in bile following BDL. (C) TBA quantification in liver bile extracts following BDL. (D) TBA quantification in serum following BDL (n=5 for 7 days and n=3-4 for 12 days, students t-test). (E) Quantification of total bilirubin levels in serum following BDL (n=5 for 2- and 7 days and n=3-4 for 12 days, students t-test). (F) Quantification of bile plugs in kidney tissue at 7 days post BDL. Quantification is based on Haematoxylin and eosin stained kidney sections, where bile plugs appear as yellow casts. Values reported as integrated density of yellow color per mm² of tissue (n=3/4, students t test). See material and methods for details.

FIG. 4. Bile acid quantification and composition in Cldn3^{+/+} and Cldn3′ liver tissue and serum, pre- and post BDL. (A) LC-MS/MS quantification of liver tissue bile acids in control- and 7 days post BDL (n=12 for control, n=8 for 7 days post BDL, students t test) (B) LC-MS/MS quantification of bile acid subtypes in control liver. Data reported as mean percentage from whole bile acid pool. (n=12, students t test comparing Cldn3^{+/+} to Cldn3^{-/-}). (C) LC-MS/MS quantification of bile acid subtypes in liver 7 days post BDL. Data reported as mean percentage from whole bile acid pool. (n=8, students t test). (D) LC-MS/MS quantification of serum bile acids in control- and 7 days post BDL (n=11 for control, n=5 for 7 days post BDL, students t test) (E) LC-MS/MS quantification of bile acid subtypes in control serum. Data reported as mean percentage from whole bile acid pool. (n=11, students t test). (F) LC-MS/MS quantification of bile acid subtypes in serum 7 days post BDL. Data reported as mean percentage from whole bile acid pool. (n=5, students t test).

FIG. 5. Lower expression of BA synthesis- and importer genes in cholestatic Cldn3^{-/-} liver. (A) Differential gene expression analysis of liver tissue at 2 days post BDL (n=3, annotated genes P<0.05) (B) RT-qPCR analysis of genes involved in bile acid synthesis. (C) Quantification of serum cholesterol levels. (D) RT-qPCR analysis of genes involved in bile acid import. (E) RT-qPCR analysis of genes involved in bile component export. (For B-E, (n=5 for 2- and 7 days and n=3-4 for 12 days, students t-test).

FIG. 6. Cldn3^{-/-} mice have increased periductal oedemaHistopathological analysis of portal fields post BDL. (A) Haematoxilin & Eosin stainings showing the periportal regions at the indicated times following BDL. (B) Scoring of periductal oedema. (n=6/4 for controls, n=5 for two- and seven days, n=4/3 for twelve days post BDL. Box plots show the mean±SD. Students t test (PV, portal vein; BD, bile duct; BI, bile infarct, HA, hepatic artery).

FIG. 7. Cldn12′-animals are not protected from cholestatic liver injury (A) Representative liver photographs and haematoxylin and eosin-stained tissue at 2 days post BDL. (B) Quantification if tissue necrosis 2 days post BDL (n=5, encircled areas highlight tissue necrosis, students t-test, ns; not significant). (C) Total bile acid quantification in liver bile extracts. (D) Total bile acid quantification in serum. (For C and D, n=3/4 in controls and n=4 at 2 days post BDL, students t-test).

FIG. 8. Claudin-3 loss protects from intrahepatic cholestasis. (A) Scheme for the acute two days ANIT challenge model. (B) Representative photographs of livers 2 days post ANIT challenge. (C) Representative images of Haematoxylin and eosin-stained liver sections 2 days post ANIT challenge (n=6, encircled areas highlight tissue necrosis). (D) Quantification of necrotic tissue area 2 days post ANIT challenge (n=6, students t-test). (E) Serum ALT, AST, ALP and bilirubin levels pre- and two days post ANIT challenge (n=6, students t-test, b.d.; below detection). (F) Liver and serum total bile acid levels 2 days post ANIT challenge (n=6, students t-test). (G) RT-qPCR expression analysis of Gsta1, Cyp4a14 and Cyp7b1 pre- and two days post ANIT challenge (n=6, students t-test). (H) Scheme for the 10 days ANIT challenge model. (I) Masson trichrome staining 10 days post ANIT gavage (encircled areas highlight high collagen staining, BI; bile infarct). (J) Quantification of collagen levels in (I) (n=8-9, students t-test) (K) Scheme for the ANIT 0.1% feeding model. (L) Representative images of Haematoxylin and eosin-stained liver sections 4 weeks post Corn oil or ANIT feeding (encircled areas highlight tissue necrosis). (M) Quantification of tissue necrosis (N) ALP, ALT, AST levels in the ANIT feeding model. (O) Serum total bilirubin and cholesterol quantification. (P) Sirius red stained liver tissues of the ANIT feeding model. Arrow indicates exemplary a region of positive stained tissue, appearing darker in the grayscale images. (Q) Quantification of the Sirius red staining (For FIG. 1-Q, n=5/6 for corn oil and n=7 for ANIT 0.1%, students t-test).

FIG. 9. GalNAc siRNA targeting of claudin-3 achieves modest amelioration of cholestatic injury (A) Scheme showing the procedure of the in vivo GalNAc siRNA BDL experiment. (B) Western blot showing claudin-3 protein levels in control or claudin-3 inhibiting-GalNAc siRNA treated mice pre- and post BDL (C; control, A; AHSA1 siRNA). (C) Results of claudin-3 mRNA and protein quantification from control or GalNAc siRNA treated mice pre- or post BDL (n=4 for mRNA, protein: control n=2, post BDL n=4). (D) Serum markers of cholestasis and liver injury after BDL and anti claudin-3 GalNAc siRNA injection. P values were calculated comparing the corresponding condition to the BDL control group (student's t test) (E) Haematoxylin and eosin-stained liver sections 2 days post BDL with or without claudin-3 GalAc siRNA injection. (F) Scheme showing the procedure of the in vivo GalNAc siRNA ANIT experiment. (G) ALP serum marker of cholestasis and liver injury after anti claudin-3 GalNAc siRNA injection and ANIT gavage. P values were calculated comparing the corresponding condition to the ANIT AHSA1 control siRNA group (student's t test). (H) Haematoxylin and eosin-stained liver sections 2 days post ANIT with or without claudin-3 GalAc siRNA injection. (For D and E: n=3 for control, n=3 for BDL+control surgery, n=4 for BDL+GalNAc siRNA groups, for G and H: n=3 for AHSA1 group, n=5 for siRNA1 group, n=4 for No ANIT control group. Students t-test).

FIG. 10. UB-VS003 anti claudin-3 antibody binding against SNU449, Hepa1-6, and human- and mouse hepatocytes (h-Heps or m-Heps, respectively), at indicated dilutions, measured by FACS.

FIG. 11. UB-VS003-GalNAc induced human claudin-3 knockdown in human hepatocytes, measured by western blot.

### DETAILED DESCRIPTION OF THE INVENTION

As outlined above, the present invention relates to a method of prevention, delay of progression or treatment of cholestasis and/or fibrosis associated with cholestasis.

Thus, in a first aspect the present invention provides an agent which inhibits the expression and/or activity of Claudin-3 for use in a method of prevention, delay of progression or treatment of cholestasis and/or fibrosis associated with cholestasis.

For the purposes of interpreting this specification, the following definitions will apply and whenever appropriate, terms used in the singular will also include the plural and vice versa. It is to be understood that the terminology used herein is for the purpose of describing particular embodiments only and is not intended to be limiting. The terms “comprising”, “having”, and “including” are to be construed as open-ended terms (i.e., meaning “including, but not limited to,”) unless otherwise noted.

Features, integers, characteristics, agents, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The term “cholestasis” as used herein refers to any condition in which the release of bile from the liver is blocked. The blockage can occur in the liver (intrahepatic cholestasis) or in the bile ducts (extrahepatic cholestasis). Physiologically, ‘cholestasis’ denotes an impairment of bile flow and failure to secrete the inorganic and organic constituents of bile. In particular, cholestasis arises from molecular and ultrastructural changes that impair the entry of small organic molecules, inorganic salts, proteins and ultimately water into the biliary space. Clinically, the physical findings of jaundice and pruritus are accompanied by elevated serum concentrations of bilirubin, bile salts and alkaline phosphatase (ALP). Cholestasis can be caused by extrahepatic issues, such as a gallstone or tumour blocking the flow of bile outside of the liver. But it can also have intrahepatic causes like viral diseases, genetic disorders and bile duct strictures. Bile constitutes the primary pathway for elimination of bilirubin, excess cholesterol (both as free cholesterol and as bile salts) and xenobiotics that are insufficiently water soluble to be excreted into urine. A fundamental driver of bile formation is hepatocellular secretion of bile salts into the canalicular space, which entrains secretion of phosphatidylcholine and cholesterol from the hepatocyte. Fluid secretion by hepatocytes and by downstream cholangiocytes lining the biliary tree jointly contributes to the several litres of bile secreted per day by the human liver. Bile facilitates the digestion and absorption of lipids from the gut. Because bile formation requires well-functioning hepatocytes and an intact biliary tree, this process is readily disrupted.

The term “fibrosis associated with cholestasis” refers to any fibrotic liver disease that is associated to cholestasis and/or that is initially caused by liver cholestasis.

The term “agent which inhibits the expression and/or activity of Claudin-3” as used herein refers to any biological or chemical agent which permits inhibition of the expression and/or inhibition of the activity of Claudin-3 e.g. by reducing or disrupting interactions of Claudin-3 or its gene with other biomolecules, such as but not limited to protein-protein interaction, ligand-receptor interaction, or protein-nucleic acid interaction. Such agents include, but are not limited to, antibodies, protein-binding agents, nucleic acid molecules, small molecules, recombinant proteins, peptides, aptamers, avimers and protein-binding derivatives, or fragments thereof. Activity of Claudin-3 can be inhibited, e.g., by antibodies binding Claudin-3 e.g. antibodies binding at least one of the extracellular domains of Claudin-3 or toxins which bind to Claudin-3. Expression of Claudin-3 can be inhibited, e.g., by a DNA targeting agent (e.g., CRISPR system, TALE, Zinc finger protein) or an RNA targeting agent (e.g., inhibitory nucleic acid molecules). Inhibition of expression of Claudin-3 comprises a decrease of expression of at least 10%, preferably of at least 40% in the presence of the agent compared to the expression of Claudin-3 without the agent. Inhibition of activity of Claudin-3 comprises a decrease of activity of at least 10%, preferably of at least 40% in the presence of the agent compared to the activity of Claudin-3 without the agent.

Claudins as referred herein are a family of integral membrane proteins that make up TJs, which are the chief intercellular junctions that act as permeability barriers and confer polarity to epithelial cells by demarcating the membrane upper and lower regions. Currently, the mammalian claudin family comprises 27 proteins, and many alternative splicing claudin proteins are expressed in various tissues. In the past decade, the crystal structures of this protein family have been gradually elucidated. Claudins are tetratransmembrane proteins, including four transmembrane domains (TM1-4), the intracellular N and C termini, and two extracellular loops (ECL1 and ECL2). ECL1 contains four β-strands and an extracellular helix (ECH), and ECL2 contains a B-strand and cell surface-exposed transmembrane 3 domain. The ECLs are involved in the formation of interactions between claudin strands and determine the gate function of claudin-based TJs by two variable regions. Claudin-3 was originally termed rat ventral prostate 1 protein (RVP1), and Clostridium perfringens enterotoxin receptor 2 (CPETR2). It was reclassified as claudin-3 on the basis of cDNA similarity with claudins-1 and -2, and antibody studies that showed it to be expressed at tight junctions. The term “Claudin-3” as used herein refers to the mammalian, preferably to the human Claudin-3 with the Uniprot (www.uniprot.org) identifier Uniprot. 015551 for the human sequence as shown in SEQ ID NO: 4: MSMGLEITGTALAVLGWLGT IVCCALPMWR VSAFIGSNII TSQNIWEGLW MNCVVQSTGQMQCKVYDSLL ALPQDLQAAR ALIVVAILLA AFGLLVALVG AQCTNCVQDD TAKAKITIVAGVLFLLAALL TLVPVSWSAN THIRDFYNPVVPEAQKREMGAGLYVGWAAAALQLLGGALLCCSCPPREKK YTATKVVYSA PRSTGPGASL GTGYDRKDYV.

The term “toxin which binds to Claudin-3” as used herein refers to a peptide that binds to Claudin-3, like CPE (Clostridium perfringens enterotoxin) or a fragment or variant thereof, such as C-terminal fragments of Clostridium perfringens enterotoxin (cCPE), for example as shown in doi: 10.1074/jbc.M111.312165. By “fragment or variant thereof” in relation to the CPE is meant that the fragment or variant (such as a cCPE analogue, derivative or mutant) is capable of binding to a extracellular domain of claudin-3, in order to inhibit claudin-3 binding to another protein. Such variants include naturally occurring allelic variants and non-naturally occurring variants. CPE is the major virulence determinant for C. perfringens.

“Antibodies”, also synonymously called “immunoglobulins” (Ig), are generally comprising four polypeptide chains, two heavy (H) chains and two light (L) chains, and are therefore multimeric proteins, or an equivalent Ig homologue thereof (e.g., a camelid nanobody, which comprises only a heavy chain, single domain antibodies (dAbs) which can be either be derived from a heavy or light chain); including full length functional mutants, variants, or derivatives thereof (including, but not limited to, murine, chimeric, humanized and fully human antibodies, which retain the essential epitope binding features of an Ig molecule, and including dual specific, bispecific, multispecific, and dual variable domain immunoglobulins; Immunoglobulin molecules can be of any class (e.g., IgG, IgE, IgM, IgD, IgA, and IgY), or subclass (e.g., IgG1, IgG2, IgG3, IgG4, IgA1, and IgA2) and allotype. The antibody used in the present invention is preferably a monoclonal antibody or a fragment thereof and comprises modified antibody formats and antibody mimetics, in particular a human or humanized antibody.

An “antibody fragment”, as used herein, relates to a molecule comprising at least one polypeptide chain derived from an antibody that is not full length, including, but not limited to (i) a Fab fragment, which is a monovalent fragment consisting of the variable light (VL), variable heavy (VH), constant light (CL) and constant heavy 1 (CHI) domains; (ii) a F(ab′) 2 fragment, which is a bivalent fragment comprising two Fab fragments linked by a disulfide bridge at the hinge region; (iii) a heavy chain portion of a Fab (Fa) fragment, which consists of the VH and CHI domains; (iv) a variable fragment (Fv) fragment, which consists of the VL and VH domains of a single arm of an antibody, (v) a domain antibody (dAb) fragment, which comprises a single variable domain; (vi) an isolated complementarity determining region (CDR); (vii) a single chain FvFragment (scFv); (viii) a diabody, which is a bivalent, bispecific antibody in which VH and VL domains are expressed on a single polypeptide chain, but using a linker that is too short to allow for pairing between the two domains on the same chain, thereby forcing the domains to pair with the complementarity domains of another chain and creating two antigen binding sites; and (ix) a linear antibody, which comprises a pair of tandem Fv segments (VH-CH1-VH-CH1) which, together with complementarity light chain polypeptides, form a pair of antigen binding regions; and (x) other non-full length portions of immunoglobulin heavy and/or light chains, or mutants, variants, or derivatives thereof, alone or in any combination.

The term “modified antibody format”, as used herein, encompasses antibody-drug-conjugates, Polyalkylene oxide-modified scFv, Monobodies, Diabodies, Camelid Antibodies, Domain Antibodies, bi- or trispecific antibodies, IgA, or two IgG structures joined by a J chain and a secretory component, shark antibodies, new world primate framework+non-new world primate CDR, IgG4 antibodies with hinge region removed, IgG with two additional binding sites engineered into the CH3 domains, antibodies with altered Fc region to enhance affinity for Fc gamma receptors, dimerised constructs comprising CH3+VL+VH, and the like.

The term “antibody mimetic”, as used herein, refers to proteins not belonging to the immunoglobulin family, and even non-proteins such as aptamers, or synthetic polymers. Some types have an antibody-like beta-sheet structure. Potential advantages of “antibody mimetics” or “alternative scaffolds” over antibodies are better solubility, higher tissue penetration, higher stability towards heat and enzymes, and comparatively low production costs.

The term “conjugation” or “conjugated”, as used herein, relates to the covalent or non-covalent binding of a molecule to another molecule. Covalent binding includes formation of a covalent bond. Non-covalent binding includes p-p (aromatic) interactions, van der Waals interactions, H-bonding interactions, and ionic interactions. A conjugate comprising covalent binding of the present invention is e.g. a N-acetylgalactosamine (GalNAc) siRNA conjugate wherein siRNA, e.g. siRNA targeting Claudin-3 is covalently bound to N-acetylgalactosamine (GalNAc), preferably covalently bound to one-to five moieties of N-acetylgalactosamine (GalNAc), more preferably covalently bound to three moieties of N-acetylgalactosamine (GalNAc). A conjugate comprising non-covalent binding of the present invention is e.g. lipid nanoparticle, a liposome or a adenovirus comprising an silencing RNA targeting Claudin-3.

The terms “nucleic acid”, “nucleic acid sequence,”, “nucleic acid molecule, “polynucleic acid sequence,” “nucleotide sequence,” and “nucleotide acid sequence” are used herein interchangeably and have the identical meaning herein and refer to preferably DNA or RNA. In some embodiments, a nucleic acid sequence is a polymer comprising or consisting of nucleotide monomers, which are covalently linked to each other by phosphodiester-bonds of a sugar/phosphate-backbone. The term “nucleic acid sequence” also encompasses modified nucleic acid sequences, such as base-modified, sugar-modified or backbone-modified etc., DNA or RNA.

The term “nucleic acid targeting a gene or mRNA”, as used herein, refers to at least one nucleic acid sequence encoding or comprising a nucleic acid like small interfering RNA (siRNA), short or small harpin RNA (shRNA), microRNA (miRNA), piwi-interacting RNA (piRNA), and long non-coding RNA (lncRNA). A small interfering RNA (siRNA) is capable of binding to a target gene or a target messenger RNA (mRNA). In some embodiments, siRNAs as used herein may be processed from a dsRNA or a shRNA. Thus the term “siRNA” as used herein, may encompass a siRNA according to the invention and a molecule, in particular a dsRNA molecule, from which a siRNA according to the invention can be generated within a mammalian cell by the RNA interference pathway. The RNA may be made by synthetic chemical and enzymatic methodology known to one of ordinary skill in the art, or by the use of recombinant technology, or may be isolated from natural sources, or by a combination thereof. The RNA may optionally comprise unnatural and naturally occurring nucleoside modifications known in the art such as e.g., N1-Methylpseudouridine also referred as methylpseudouridine. In some embodiments, the nucleic acid targeting a gene or mRNA of the present invention comprises multiple copies of siRNAs that can target one mRNA.

The term “siRNA binding Claudin-3”, as used herein, relates to a small interfering RNA (siRNA) capable of binding to a target messenger RNA (mRNA) of Claudin-3. siRNAs as used herein may comprise a double-stranded RNA (dsRNA) region, a hairpin structure, a loop structure, or any combinations thereof. In some embodiments, siRNAs may comprise at least one shRNA, at least one dsRNA region, or at least one loop structure. In some embodiments, siRNAs may be processed from a dsRNA or an shRNA. In some embodiments, siRNAs may be processed or cleaved by an endogenous protein, such as DICER, from an shRNA. In some embodiments, a hairpin structure or a loop structure may be cleaved or removed from an siRNA. For example, a hairpin structure or a loop structure of an shRNA may be cleaved or removed. In some embodiments, RNAs described herein may be made by synthetic, chemical, or enzymatic methodology known to one of ordinary skill in the art, made by recombinant technology known to one of ordinary skill in the art, or isolated from natural sources, or made by any combinations thereof. The RNA may comprise modified or unmodified nucleotides or mixtures thereof, e.g., the RNA may optionally comprise chemical and naturally occurring nucleoside modifications known in the art. In some embodiments, siRNA may comprise a nucleic acid sequence comprising a sense siRNA strand. In some embodiments, siRNA may comprise a nucleic acid sequence comprising an anti-sense siRNA strand. In some embodiments, siRNA may comprise a nucleic acid sequence comprising a sense siRNA strand and a nucleic acid sequence comprising an anti-sense siRNA strand.

The term “compound which facilitates delivery of the agent to the liver” or “compound which facilitates delivery of the siRNA to the liver” as used herein, relates to e.g. a sugar, a lipid nanoparticle, a liposome, or a adenovirus. A compound which facilitates delivery of the siRNA to the liver is e.g. N-acetylgalactosamine (GalNAc), which is preferred.

The terms “individual,” “subject” or “patient” are used herein interchangeably. In certain embodiments, the subject is a mammal. Mammals include, but are not limited to primates (including human and non-human primates). In a preferred embodiment, the subject is a human.

The term “pharmaceutically acceptable carrier” as used herein refers to carriers that are suitable for use with humans and/or animals without undue adverse side effects (such as toxicity, irritation, and allergic response) commensurate with a reasonable benefit/risk ratio. “Carriers” can be solvents, suspending agents or vehicles, for delivering the instant agents to a subject.

The term “about” as used herein refers to +/−10% of a given measurement.

Thus, in a first aspect the present invention provides an agent which inhibits the expression and/or activity of Claudin-3 for use in a method of prevention, delay of progression or treatment of cholestasis and/or fibrosis associated with cholestasis.

Agent which Inhibits the Expression and/or Activity of Claudin-3

In one embodiment the agent which inhibits the expression and/or activity of Claudin-3 is a siRNA targeting Claudin-3 or an antibody or a fragment thereof which binds to Claudin-3.

In one embodiment the agent which inhibits the expression and/or activity of Claudin-3 is an agent which inhibits the expression of Claudin-3.

In a further embodiment the agent which inhibits the expression of Claudin-3 is a nucleic acid targeting a gene or mRNA coding for Claudin-3.

Nucleic acids targeting a gene coding for Claudin-3 or targeting a mRNA coding for Claudin-3 can be e.g. at least one nucleic acid sequence encoding or comprising a nucleic acid like small interfering RNA (siRNA), short or small harpin RNA (shRNA), microRNA (miRNA), piwi-interacting RNA (piRNA), and long non-coding RNA (lncRNA). Preferably the agent which inhibits the expression of Claudin-3 is a small interfering RNA (siRNA) targeting Claudin-3 i.e. a siRNA capable of binding to a gene encoding Claudin-3 or a target messenger RNA (mRNA) encoding Claudin-3, more preferably a small interfering RNA (siRNA) capable of binding to a target messenger RNA (mRNA) encoding Claudin-3. Preferably the siRNA targeting Claudin-3 comprises 10-50, more preferably 15-40, even more preferably 17-24 nucleotides. In certain embodiments, the siRNA according to the invention comprises 17, 18, 19, 20, 21, 22, 23, 24, 25, 26 or 27 nucleotides.

In an even more preferred embodiment, the siRNA targeting Claudin-3 comprises the sequence as shown in SEQ ID NO:1 (sense strand), siRNA comprising the sequence as shown in SEQ ID NO: 2 (sense strand), siRNA which is at least 95% identical, more preferably 96%, 97%, 98%, 99% or 100% identical to the siRNA comprising the sequence as shown in SEQ ID NO: 1 and siRNA which is at least 95% identical, more preferably 96%, 97%, 98%, 99% or 100% identical to the siRNA comprising the sequence as shown in SEQ ID NO: 2.

In a preferred embodiment, the siRNA targeting Claudin-3 is characterized by a sequence reverse complementary to SEQ ID NO: 1 or by a sequence reverse complementary to SEQ ID NO: 2. Thus in one embodiment the sequence reverse complementary to SEQ ID NO:1 is the sequence as shown in SEQ ID NO: 5 (anti-sense strand) and the sequence reverse complementary to SEQ ID NO:2 is the sequence as shown in SEQ ID NO: 6 (anti-sense strand).

In a particular preferred embodiment the siRNA targeting Claudin-3 is selected from the group consisting of siRNA comprising the sequence as shown in any of SEQ ID NOs: 1, 2, 5, 6 or 33-168 or siRNA which is at least 95% identical, more preferably 96%, 97%, 98%, 99% or 100% identical to the siRNA comprising the sequence as shown in any of SEQ ID NO: 1, 2, 5, 6 or 33-168.

In a more particular preferred embodiment the siRNA targeting Claudin-3 is selected from the group consisting of siRNA comprising the sequence as shown in any of SEQ ID NOs: 33-168 or siRNA which is at least 95% identical, more preferably 96%, 97%, 98%, 99% or 100% identical to the siRNA comprising the sequence as shown in any of SEQ ID NO: 33-168.

In an even more particular preferred embodiment the siRNA targeting Claudin-3 selected from the group consisting of siRNA comprising the sequence as shown in any of SEQ ID NOs: 33-100 or siRNA which is at least 95% identical, more preferably 96%, 97%, 98%, 99% or 100% identical to the siRNA comprising the sequence as shown in any of SEQ ID NO: 33-100.

In a further particular preferred embodiment the siRNA targeting Claudin-3 is selected from the group consisting of siRNA comprising the sequence as shown in any of SEQ ID NOs: 1, 2, 33-66 or 101-134 siRNA which is at least 95% identical, more preferably 96%, 97%, 98%, 99% or 100% identical to the siRNA comprising the sequence as shown in any of SEQ ID NO: 1, 2, 33-66 or 101-134.

In a further more particular preferred embodiment the siRNA targeting Claudin-3 is selected from the group consisting of siRNA comprising the sequence as shown in any of SEQ ID NOs: 33-66 or 101-134 or siRNA which is at least 95% identical, more preferably 96%, 97%, 98%, 99% or 100% identical to the siRNA comprising the sequence as shown in any of SEQ ID NO: 33-66 or 101-134.

In a further even particular preferred embodiment the siRNA targeting Claudin-3 selected from the group consisting of siRNA comprising the sequence as shown in any of SEQ ID NOs: 33-66 or siRNA which is at least 95% identical, more preferably 96%, 97%, 98%, 99% or 100% identical to the siRNA comprising the sequence as shown in any of SEQ ID NO: 33-66.

In the context of the present specification, the terms sequence identity and percentage of sequence identity refer to the values determined by comparing two aligned sequences. Methods for alignment of sequences for comparison are well-known in the art. Alignment of sequences for comparison may be conducted by the local homology algorithm of Smith and Waterman, Adv. Appl. Math. 2:482 (1981), by the global alignment algorithm of Needleman and Wunsch, J. Mol. Biol. 48:443 (1970), by the search for similarity method of Pearson and Lipman, Proc. Nat. Acad. Sci. 85:2444 (1988) or by computerized implementations of these algorithms, including, but not limited to: CLUSTAL, GAP, BESTFIT, BLAST, FASTA and TFASTA. Software for performing BLAST analyses is publicly available, e.g., through the National Center for Biotechnology-Information (http://blast.ncbi.nlm.nih.gov/).

In one embodiment the agent which inhibits the expression and/or activity of Claudin-3 is an agent which inhibits the activity of Claudin-3.

In a further embodiment the agent which inhibits the activity of Claudin-3 is selected from the group consisting of an antibody or a fragment thereof which binds to Claudin-3 and a toxin which binds to Claudin-3. Preferably the toxin which binds to Claudin-3 is Clostridium perfringens enterotoxin (CPE) or a fragment or variant thereof.

In a preferred embodiment the agent which inhibits the activity of Claudin-3 is an antibody or a fragment thereof which binds to Claudin-3, preferably a monoclonal antibody or a fragment thereof which binds to Claudin-3, even more preferably an antibody or a fragment thereof which binds to an extracellular domain of Claudin-3, in particular a monoclonal antibody or a fragment thereof which binds to an extracellular domain of Claudin-3. In a further preferred embodiment the antibody or a fragment thereof which binds to Claudin-3 is a human or humanized antibody, more preferably a monoclonal human antibody. In a particular preferred embodiment the antibody or a fragment thereof which binds to Claudin-3 comprises a light chain comprising the amino acid sequence as shown in SEQ ID NO: 169 and/or a heavy chain comprising the amino acid sequence as shown in SEQ ID NO: 170.

In a more particular preferred embodiment the agent which inhibits the activity of Claudin-3 is an antibody or a fragment thereof, preferably a monoclonal antibody or a fragment thereof which binds to the extracellular loop 1 and/or 2 (ECL1 and/or ECL2) of claudin-3.

In one embodiment the agent which inhibits the expression and/or activity of Claudin-3 is conjugated to a compound which facilitates delivery of the agent to the liver. A compound which facilitates delivery of the agent to the liver can be e.g. N-acetylgalactosamine (GalNAc), a lipid nanoparticle, a liposome or an adenovirus.

In a preferred embodiment the antibody or a fragment thereof which binds to Claudin-3 is conjugated to a compound which facilitates delivery of the antibody to the liver, even more preferably, the antibody or a fragment thereof which binds to Claudin-3 is a N-acetylgalactosamine (GalNAc) antibody conjugate.

In a preferred embodiment the siRNA targeting Claudin-3 is conjugated to a compound which facilitates delivery of the siRNA to the liver. In a more preferred embodiment the agent is a N-acetylgalactosamine (GalNAc) siRNA conjugate i.e. a conjugate of a siRNA targeting Claudin-3 and N-acetylgalactosamine (GalNAc). Usually such a conjugate comprises 1-5, preferably 3 moieties of GalNAc covalently bound to one moiety of siRNA.

### Compositions

As outlined above, the invention also relates to a composition comprising an agent which inhibits the expression and/or activity of Claudin-3 and optionally a pharmaceutically acceptable carrier for use in a method of prevention, delay of progression or treatment of cholestasis and/or fibrosis associated with cholestasis.

The term “composition” refers usually to a fixed-dose combination (FDC) that includes the compound which inhibits the expression and/or activity of Claudin-3 in a single dosage form, having a predetermined combination of respective dosages.

The composition further may be used as add-on therapy. As used herein, “add-on” or “add-on therapy” means an assemblage of reagents for use in therapy, the subject receiving the therapy begins a first treatment regimen of one or more reagents prior to beginning a second treatment regimen of one or more different reagents in addition to the first treatment regimen, so that not all of the reagents used in the therapy are started at the same time.

The amount of the agent which inhibits the expression and/or activity of Claudin-3 to be administered will vary depending upon factors such as the particular agent, disease condition and its severity, according to the particular circumstances surrounding the case, including, e.g., the specific agent which inhibits the expression and/or activity of Claudin-3 being administered, the route of administration, the condition being treated, the target area being treated, and the subject or host being treated.

In one embodiment, the invention provides a composition comprising an agent which inhibits the expression and/or activity of Claudin-3, wherein said agent which inhibits the expression and/or activity of Claudin-3 is present in a therapeutically effective amount.

The expression “effective amount” or “therapeutically effective amount” as used herein refers to an amount capable of invoking one or more of the following effects in a subject receiving the composition of the present invention: (i) dilution and/or—detoxification of the bile, which contains a higher amount of water than before the start of the therapy (ii) decrease of the concentration of liver- and/or blood bile acids (iii) decreased levels of the cholestasis marker alkaline phosphatase (ALP) in the blood (iv) less tissue necrosis in the liver (v) improved quality of life (vi) longer symptom free survival (vii) longer liver transplant free survival (viii) ameliorated cholestasis symptoms like jaundice or pruritus (ix) increased survival rate of patients with cholestasis and/or fibrosis associated with cholestasis.

Determination of a therapeutically effective amount is well within the capability of those skilled in the art, especially in light of the detailed disclosure provided herein.

In one embodiment, the invention provides a composition comprising agent which inhibits the expression and/or activity of Claudin-3, wherein the amount of said agent which inhibits the expression and/or activity of Claudin-3 in the composition is from about 0.1 mg to about 10 g.

### Formulations and Modes of Administration

A composition according to the invention is, preferably, suitable for subcutaneous, intra-venous or oral administration to a subject and comprises a therapeutically effective amount of the active ingredient and one or more suitable pharmaceutically acceptable carrier.

Compositions, like pharmaceutical compositions, which are preferred can be formulated in a conventional manner using one or more pharmaceutically acceptable inactive ingredients, like pharmaceutical carriers that facilitate processing of the active compounds into preparations that can be used pharmaceutically. A proper formulation is dependent upon the route of administration chosen and a summary of pharmaceutical compositions can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999). If not indicated otherwise, a composition according to the invention is prepared in a manner known per se, e.g. by means of conventional mixing, granulating, coating, dissolving or lyophilizing processes. In preparing a composition for an oral dosage form, any of the usual pharmaceutical media may be employed, for example water, glycols, oils, alcohols, carriers, such as starches, sugars, or microcrystalline cellulose, diluents, granulating agents, lubricants, binders, disintegrating agents and the like.

In one embodiment, the invention provides a composition comprising an agent which inhibits the expression and/or activity of Claudin-3 and at least one pharmaceutically acceptable carrier, wherein the composition is a solution.

In one embodiment, the invention provides a composition comprising an agent which inhibits the expression and/or activity of Claudin-3 and at least one pharmaceutically acceptable carrier, wherein the composition is, wherein the composition is a tablet or a capsule, preferably a tablet.

### Dosing Regimen

An exemplary treatment regime entails administration once daily, twice daily, three times daily, every second day, twice per week, once per week. The composition of the invention is usually administered on multiple occasions. Intervals between single dosages can be, for example, less than a day, daily, every second day, twice per week, or weekly. The composition of the invention may be given as a continuous uninterrupted treatment. The composition of the invention may also be given in a regime in which the subject receives cycles of treatment interrupted by a drug holiday or period of non-treatment. Thus, the composition of the invention may be administered according to the selected intervals above for a continuous period of one week or a part thereof, for two weeks, for three weeks, for four weeks, for five weeks or for six weeks and then stopped for a period of one week, or a part thereof, for two weeks, for three weeks, for four weeks, for five weeks, or for six weeks. The composition of the treatment interval and the non-treatment interval is called a cycle. The cycle may be repeated one or more times. Two or more different cycles may be used in combination for repeating the treatment one or more times. Intervals can also be irregular as indicated by measuring blood levels of said agent which inhibits the expression and/or activity of Claudin-3 in the patient. In a preferred embodiment, the composition according to the invention is administered once daily. In an exemplary treatment the agent which inhibits the expression and/or activity of Claudin-3 can be administered from 0.1 mg-10 g per day.

Using an Agent which Inhibits the Expression and/or Activity of Claudin-3 or a Composition Thereof to Prevent, Delay of Progression or Treat Cholestasis and/or Fibrosis Associated with Cholestasis

The present invention provides an agent which inhibits the expression and/or activity of Claudin-3 or a composition thereof as described herein, for use in a method of prevention, delay of progression or treatment cholestasis and/or fibrosis associated with cholestasis in a subject.

Also provided is the use of an agent which inhibits the expression and/or activity of Claudin-3 or a composition thereof as described herein for the manufacture of a medicament for the prevention, delay of progression or treatment of cholestasis and/or fibrosis associated with cholestasis in a subject.

Also provided is the use of an agent which inhibits the expression and/or activity of Claudin-3 or a composition thereof as described herein for the prevention, delay of progression or treatment of cholestasis and/or fibrosis associated with cholestasis in a subject.

Also provided is a method of prevention, delay of progression or treatment of cholestasis and/or fibrosis associated with cholestasis in a subject, comprising administering to said subject a therapeutically effective amount of an agent which inhibits the expression and/or activity of Claudin-3 or a composition thereof, as described herein.

The terms “treatment”/“treating” as used herein includes: (1) delaying the appearance of clinical symptoms of the state, disorder or condition developing in an animal, particularly a mammal and especially a human, that may be afflicted with or predisposed to the state, disorder or condition but does not yet experience or display clinical or subclinical symptoms of the state, disorder or condition; (2) inhibiting the state, disorder or condition (e.g. arresting, reducing or delaying the development of the disease, or a relapse thereof in case of maintenance treatment, of at least one clinical or subclinical symptom thereof); and/or (3) relieving the condition (i.e. causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

As used herein, “delay of progression” means increasing the time from symptoms to worsening of symptoms of cholestasis and/or fibrosis associated with cholestasis and includes reversing or inhibition of disease progression. “Inhibition” of disease progression or disease complication in a subject means preventing or reducing the disease progression and/or disease complication in the subject.

Preventive treatments comprise prophylactic treatments. In preventive applications, the composition of the invention is administered to a subject suspected of having, or at risk for developing cholestasis and/or fibrosis associated with cholestasis. In therapeutic applications, the composition is administered to a subject such as a patient already suffering from cholestasis and/or fibrosis associated with cholestasis s, in an amount sufficient to cure or at least partially arrest the symptoms of the disease. Amounts effective for this use will depend on the severity and course of the disease, previous therapy, the subject's health status and response to the drugs, and the judgment of the treating physician.

In the case wherein the subject's condition does not improve, the composition of the invention may be administered chronically, which is, for an extended period of time, including throughout the duration of the subject's life in order to ameliorate or otherwise control or limit the symptoms of the subject's disease or condition.

In the case wherein the subject's status does improve, the composition may be administered continuously; alternatively, the dose of drugs being administered may be temporarily reduced or temporarily suspended for a certain length of time (i.e., a “drug holiday”).

Once improvement of the patient's condition has occurred, a maintenance dose of the composition of the invention is administered if necessary. Subsequently, the dosage or the frequency of administration, or both, is optionally reduced, as a function of the symptoms, to a level at which the improved disease is retained.

In one embodiment cholestasis is intrahepatic cholestasis or extrahepatic cholestasis, preferably intrahepatic cholestasis. In a preferred embodiment cholestasis is intrahepatic cholestasis wherein the intrahepatic cholestasis is acute intrahepatic cholestasis or chronic intrahepatic cholestasis. In an even more preferred embodiment cholestasis is chronic intrahepatic cholestasis, preferably chronic intrahepatic cholestasis selected from the group consisting of primary biliary cholangitis (PBC) and primary sclerosing cholangitis (PSC).

In one embodiment cholestasis is a disease selected from the group consisting of obstructive cholestasis, nonobstructive cholestasis, bile duct diseases and defects in biliary function, more preferably bile duct diseases, in particular malignant bile duct obstructions.

In a preferred embodiment cholestasis and/or fibrosis associated with cholestasis are selected from the group consisting of chronic intrahepatic cholestasis and bile duct diseases.

In a more preferred embodiment cholestasis and/or fibrosis associated with cholestasis are selected from the group consisting of primary biliary cholangitis (PBC), primary sclerosing cholangitis (PSC) and malignant bile duct obstructions.

Obstructive cholestasis can be associated with or can be caused by gallstones, pancreatic cancer, bile duct adenocarcinoma, stricture of the common bile duct, biliary atresia like extrahepatic biliary atresia, common bile duct obstruction caused by like biliary sludge or gallstones, choledochal cyst with biliary sludge and/or inspissated bile/mucous plug.

Nonobstructive cholestasis can be associated with or can be caused by viral hepatitis (hepatitis B hepatitis C), toxic effects of drugs or nutrition, paraneoplastic syndrome like Hodgkin lymphoma, Wilson disease, familial cholestatic syndromes, intrahepatic cholestasis of pregnancy, infiltrative disorders like amyloidosis, ormetastatic cancer, and/or cirrhosis (any cause), bacterial infection like infections with gram-negative enteric bacteraemia, syphilis, listeria, toxoplasma, or sepsis or endotoxaemia caused by bacterial infection, viral infections with cytomegalovirus, herpesvirus (includes simplex, zoster, parvovirus B19, adenovirus), rubella, reovirus, and enteroviruses.

Bile duct diseases can be associated with or can be caused by primary biliary cholangitis, primary sclerosing cholangitis, Graft-versus-host disease (acute and chronic), transplant rejection (acute and chronic), Transplant: infarction of the biliary tree secondary to hepatic artery obstruction, vanishing bile duct syndromes caused by toxic effects of drugs such as e.g. ibuprofen or chlorpromazine.

Defects in biliary function can be associated with or can be caused by alpha-1-antitrypsin storage disease, cystic fibrosis, galactosaemia, tyrosinaemia, fatty acid oxidation defects, lipid storage disorders, glycogen storage disorders, peroxisomal disorders, bile acid biosynthetic defects, PFIC1, PFIC2, PFIC3 (PFIC, Progressive familial intrahepatic cholestasis), pucity of bile ducts like Alagille syndrome or nonsyndromic paucity of bile duct syndromes.

In a preferred embodiment cholestasis is a disease selected from the group consisting of obstructive cholestasis associated with or caused by stricture of the common bile duct, biliary atresia like extrahepatic biliary atresia and/or common bile duct obstruction caused by like biliary sludge or gallstones, preferably obstructive cholestasis associated with or caused by stricture of the common bile duct; nonobstructive cholestasis associated with or caused by toxic effects of drugs, Wilson disease and/or familial cholestatic syndromes; bile duct diseases associated with or caused by primary biliary cholangitis, primary sclerosing cholangitis and/or vanishing bile duct syndromes caused by toxic effects of drugs such as e.g. ibuprofen or chlorpromazine; and defects in biliary function associated with or caused by bile acid biosynthetic defects, PFIC1, PFIC2, PFIC3.

In a more preferred embodiment cholestasis is obstructive cholestasis. In an even more preferred embodiment cholestasis is obstructive cholestasis associated with or caused by stricture of the common bile duct or biliary atresia like extrahepatic biliary atresia.

In one embodiment fibrosis associated with cholestasis is a disease where the fibrosis is at least in part induced by an unresolved cholestasis, preferably fibrosis associated with cholestasis is a liver disease where the fibrosis is at least in part induced by a unresolved cholestasis. Fibrosis associated with cholestasis is usually selected from the diseases or conditions selected from the group consisting of fibrosis due to hepatitis A Virus infection associated with cholestasis, fibrosis due to hepatitis B Virus infection associated with cholestasis, fibrosis due to hepatitis C Virus infection associated with cholestasis, fibrosis due to Epstein-Barr-Virus infection associated with cholestasis, fibrosis due to hemochromatosis associated with cholestasis, fibrosis due to autoimmune hepatitis associated with cholestasis, fibrosis due to secondary biliary cirrhosis associated with cholestasis, fibrotic due to alcoholic liver disease associated with cholestasis and/or clinical jaundice, fibrosis due to drug induced liver disease associated with cholestasis, fibrosis caused by metabolic syndromes, e.g., impaired glucose metabolism, fat accumulation or impaired lipid metabolism, associated with cholestasis, liver fibrosis due to Non-alcoholic fatty liver disease (NAFLD) that associated with cholestasis, liver fibrosis caused by dysregulated iron- and copper homeostasis, associated with non-alcoholic fatty liver disease associated with cholestasis, liver fibrosis due to Non-alcoholic steatohepatitis (NASH) associated with cholestasis, fibrosis due to radiation induced liver disease associated with cholestasis, fibrosis due to Wilson disease associated with cholestasis, fibrosis due to al-Antitrypsin deficiency associated with cholestasis, and fibrosis due to Sinusoidal obstruction syndrome associated with cholestasis. Preferably, the present invention provides a treatment for fibrosis causing metabolic diseases like NAFLD or NASH, in particular a treatment for fibrosis causing liver diseases using the agent as described herein.

In a further aspect the present invention provides a dosage form for the prevention, delay of progression or treatment cholestasis and/or fibrosis associated with cholestasis, comprising an agent which inhibits the expression or activity of Claudin-3 or a composition comprising said agent, and optionally a pharmaceutically acceptable carrier.

In a further aspect, the present invention provides a siRNA targeting Claudin-3. In a preferred embodiment the present invention provides siRNAs targeting Claudin-3 selected from the group consisting of siRNA comprising the sequence as shown in SEQ ID NO:1, siRNA comprising the sequence as shown in SEQ ID NO: 2, siRNA which is at least 95% identical, more preferably 96%, 97%, 98%, 99% or 100% identical to the siRNA comprising the sequence as shown in SEQ ID NO:1 and siRNA which is at least 95% identical, more preferably 96%, 97%, 98%, 99% or 100% identical to the siRNA comprising the sequence as shown in SEQ ID NO: 2.

In a further embodiment the present invention provides siRNAs targeting Claudin-3 selected from the group consisting of siRNA comprising the sequence as shown in SEQ ID NO:5, siRNA comprising the sequence as shown in SEQ ID NO: 6, siRNA which is at least 95% identical, more preferably 96%, 97%, 98%, 99% or 100% identical to the siRNA comprising the sequence as shown in SEQ ID NO:5 and siRNA which is at least 95% identical, more preferably 96%, 97%, 98%, 99% or 100% identical to the siRNA comprising the sequence as shown in SEQ ID NO: 6.

In a preferred embodiment the present invention provides siRNAs targeting Claudin-3 selected from the group consisting of siRNA comprising the sequence as shown in any of SEQ ID NOs: 1, 2, 5, 6 or 33-168 or siRNA which is at least 95% identical, more preferably 96%, 97%, 98%, 99% or 100% identical to the siRNA comprising the sequence as shown in any of SEQ ID NO: 1, 2, 5, 6 or 33-168.

In a more preferred embodiment the present invention provides siRNAs targeting Claudin-3 selected from the group consisting of siRNA comprising the sequence as shown in any of SEQ ID NOs: 33-168 or siRNA which is at least 95% identical, more preferably 96%, 97%, 98%, 99% or 100% identical to the siRNA comprising the sequence as shown in any of SEQ ID NO: 33-168.

In an even more preferred embodiment the present invention provides siRNAs targeting Claudin-3 selected from the group consisting of siRNA comprising the sequence as shown in any of SEQ ID NOs: 33-100 or siRNA which is at least 95% identical, more preferably 96%, 97%, 98%, 99% or 100% identical to the siRNA comprising the sequence as shown in any of SEQ ID NO: 33-100.

In a particular embodiment the present invention provides siRNAs targeting Claudin-3 is selected from the group consisting of siRNA comprising the sequence as shown in any of SEQ ID NOs: 1, 2, 33-66 or 101-134 or siRNA which is at least 95% identical, more preferably 96%, 97%, 98%, 99% or 100% identical to the siRNA comprising the sequence as shown in any of SEQ ID NO: 1, 2, 33-66 or 101-134.

In a more particular embodiment the present invention provides siRNAs targeting Claudin-3 is selected from the group consisting of siRNA comprising the sequence as shown in any of SEQ ID NOs: 33-66 or 101-134 or siRNA which is at least 95% identical, more preferably 96%, 97%, 98%, 99% or 100% identical to the siRNA comprising the sequence as shown in any of SEQ ID NO: 33-66 or 101-134.

In an even more particular embodiment the present invention provides siRNAs targeting Claudin-3 selected from the group consisting of siRNA comprising the sequence as shown in any of SEQ ID NOs: 33-66 or siRNA which is at least 95% identical, more preferably 96%, 97%, 98%, 99% or 100% identical to the siRNA comprising the sequence as shown in any of SEQ ID NO: 33-66.

In a further aspect, the present invention provides an antibody or a fragment thereof which binds to Claudin-3. In one embodiment the antibody or a fragment thereof which binds to Claudin-3 is a human antibody. In a preferred embodiment the antibody or a fragment thereof which binds to Claudin-3 comprises a light chain comprising the amino acid sequence as shown in SEQ ID NO: 169 and/or a heavy chain comprising the amino acid sequence as shown in SEQ ID NO: 170. In a further embodiment the antibody or a fragment thereof which binds to Claudin-3 is conjugated to a compound which facilitates delivery of the antibody to the liver. In a further preferred embodiment the antibody or a fragment thereof which binds to Claudin-3 is a N-acetylgalactosamine (GalNAc) antibody conjugate. A particular preferred antibody is a human antibody which binds to Claudin-3, more particular a human antibody which binds to Claudin-3 which comprises a light chain comprising the amino acid sequence as shown in SEQ ID NO: 169 and/or a heavy chain comprising the amino acid sequence as shown in SEQ ID NO: 170 and which is a N-acetylgalactosamine (GalNAc) antibody conjugate.

### EXAMPLES

The present examples are intended to illustrate the present invention without restricting it.

### Materials and Methods

### Animal Housing and Ethical Approval

12-18 week old C57BL/6J background CLDN3^{+/+} or CLDN3^{-/-} mice (˜18-22 g) were kept in a 12 h light-cycle controlled room and fed standard diet laboratory diet. All mouse experiments were performed with the approval of the Veterinary Office of the Canton Bern (permit BE37/20), according to the guidelines of good animal practice as de fined by the Office of Laboratory Animal Welfare and adhering to the standards of the national centre for the replacement, refinement and reduction of animals in research guidelines (https://www.nc3rs.org.uk/arrive-guidelines).

### Bile Flow, and FITC Dextran Passage to Bile

The integrity of the paracellular barrier of hepatocyte tight junction was measured as previously described¹⁴. Quantification of the blood- to bile passage of FITC dextran (40 kDa) was measured. In anaestetized mice, the common bile duct was ligated, and the gallbladder was canulated. Bile was subsequently collected for 20 minutes to determine the bile flow rate. Then, 400 μl of FITC dextran (40 kDa, 25 mg/ml in saline) were injected into the inferior vena cava. Bile was collected from the canulation for 40 minutes, in 3-minute fractions. The liver was surgically removed and weighted. Bile was diluted 1/50 in water, and FITC fluorescence was measured at excitation 489 nm/emission 534 nm, using a Tecan Spark® plate reader. Data is reported as relative fluorescent units (RFU) per μl bile per minute per g liver.

### Quantification of the Uptake of FITC Dextran

In anaestetized mice, the common bile duct was ligated and 300 μl of FITC dextran (40 kDa, 6.25 mg/ml in saline) were injected via the tail vein. FITC fluorescence was acquired in a timeseries imaging using the IVIS® Spectrum system (Perkin Elmer). One image was acquired each 30 seconds, for a total of 45 minutes. Regions of interest with same area size were drawn around the liver. The FITC signal intensity was quantified in each image using the device software. Data is reported as total radiant efficiency [p/s]/[μW/cm²]. Using the same experimental setup, mouse livers were harvested at 20 minutes post FITC dextran (40 kDa) injection and cryosections (25 μm) were made. Sections were stained with DAPI (D9542, diluted 1:2000; Sigma-Aldrich) for 15 minutes and mounted with fluorescence mounting medium (H-1000; Vectorlabs, Burlin-game, CA). Confocal Z-stack images were acquired in periportal liver regions, to visualize the uptake of FITC Dextran (LSM 710; Zeiss, 20× objective, Oberkochen, Germany). FITC signal was quantified in complete Z-stacks of the same area and thickness, using ImageJ software (version 1.48; National Institutes of Health, Bethesda, MD). Data reported as total signal intensity (integrated density).

### Bile Duct Ligation

BDL operations were performed as described previously¹⁵. In brief, anesthetized mice were given analgesia with buprenorphine (0.1 ug/g body weight, Temgesic®) and a transverse laparotomy was performed. The common bile duct was exposed and double ligated with 7-0 silk (Sofsilk™, Covidien/Medtronic, Dublin, Ireland) and cut in between the ligatures. The laparotomy was closed with 6-0 Prolene® suture (Ethicon, Bridgewater, New Jersey, United states). Buprenorphine analgesia was repeated if indicated based on animal health scoring.

### Hematoxylin & Eosin Staining

Liver paraffin sections were deparaffinized stained with Haematoxylin (Merck, Cat. #HX43078349) for six minutes, and differentiated in HCL-ALC (1:1) performing three dips. Slides were incubated in Eosin (Fluka Chemical Corp, Cat. #45240) for 3 minutes followed by dehydration and mounting with Eukitt® (Kindler).

### Sirius Red Staining and Quantification

Slides were dewaxed, hydratated and placed in pre-warmed Bouin's fixative (Sigma-Aldrich, Cat. No. HT-10-1-32) for 20 minutes at 54° C. After a wash in running water, slides were with Weigert's iron hematoxylin (Sigma-Aldrich, Cat. No. HAT 10-79) for 5 minutes. After a wash with running tap water for 5 minutes, slides were destained using HCl-EtOH (5 ml HCl 37% in 1 liter of EtOH 70%), followed by another wash in water for 5 minutes. Slides were then stained picro-sirius (0.5 g in 1 L of Bouin's solution, Sigma-Aldrich Cat. No. 365548) for 5 minutes. Followed by two 5-minute washes in 0.5% acetic acid diluted in water, slides were dehydrated and mounted with Eukitt (Kindler). Images were acquired using a slide scanner (Panoramic 250 Flash III, 3DHISTECH) and the stainings were quantified with ImageJ (v1.52n, National Institutes of Health, Bethesda, MD). Data analysis was performed blinded. For each mouse liver, twelve randomly chosen regions of 10× magnified images of the same size were quantified. Unmodified images only were used, and all five liver lobes were covered within the analysis. The Sirius red signal was separated from the other channels using the deconvolution setting “Azon-Mallory”. In the red channel, the threshold was set. After conversion of the threshold adjusted image into an 8-bit image, the signal intensity was measured as integrated density. Results are represented as average integrated density per mm².

### Masson Trichrome Staining and Quantification

Masson trichrome staining. Paraffin-embedded liver tissue was dewaxed and placed in Bouin's fixative (HT10-1-32; Sigma-Aldrich) at 56° C. for 10 minutes. After washing slides in tap water and distilled H 2 O, slides were stained with hematoxylin (HT10-79; Sigma-Aldrich) for 5 minutes. After washing in running tap water and distilled H₂O, slides were destained once with HCl-alcohol (1:1) and rinsed again in distilled H 2 O. Next, slides were put in Biebrich scarlet-scid fuchsin (HT151-250ML; Sigma-Aldrich) diluted 1:2 in 1% acetic acid (K45741563 425; Dr. Grogg Chemie, Stettlen, Switzerland) for 1 minute. Slides were rinsed and stained with phosphomolybdic-phosphotungstic acid (HT153-250ML and HT152-250ML; Sigma) 1:1 for 5 minutes. Slides then were stained with Aniline Blue (HT154-250ML; Sigma) for 20 minutes. After a last rinse, slides were put in 0.75% acetic acid, dehydrated, and mounted with Eukitt (Kindler). Images were acquired using a slide scanner (Panoramic 250 Flash III, 3DHISTECH) and the stainings were quantified with ImageJ (v1.52n, National Institutes of Health, Bethesda, MD). For each mouse liver, six randomly chosen regions of 7× magnified images of the same size were quantified. Unmodified images only were used. The blue collagen signal was separated from the other channels using the deconvolution setting “Masson Trichrome”. In the blue channel, the threshold was set. After conversion of the threshold adjusted image into an 8-bit image, the signal intensity was measured as integrated density. Results are represented as average integrated density.

### Quantification of Tissue Necrosis

Sections of liver tissue prior and after BDL were stained with haematoxylin/eosin and images were taken using a bright-field microscope (Panoramic 250 Flash III, 3DHISTECH). Sections for quantification covered an average surface of 75 mm² per individual. Necrotic parenchymal areas were manually encircled. Data is reported as percentage of necrotic area over the entire section.

### Measurement of Bilirubin ALT, AST, ALP, Cholesterol and Total Bilirubin in Serum

The liver injury markers ALT and AST were measured on a Cobas 8000 modular analyzer using the module C502 (Roche, Switzerland). ALP and total bilirubin likewise were measured on the Cobas 8000, using the module C702 (Roche, Switzerland). All measurements were performed following the manufacturer's instructions.

### Quantification of Kidney Bile Plugs

7 days post BDL kidneys were sectioned entirely, and tissue was stained with haematoxylin/eosin. Images were taken using a bright-field microscope (Panoramic 250 Flash III, 3DHISTECH). ImageJ software was used to deconvolute the yellow-colored bile plugs from the pink haematxilin/eosin staining. The yellow color was transformed to a black and white 8-bit image, and the signal intensity was measured as integrated density. At least 10 different images, each covering 3,034 mm² of kidney tissue, were taken per mouse and results were averaged. Data is reported as integrated density per mm² kidney tissue.

### Scoring of Liver Periductal Oedema

Entire haematoxilin and eosin-stained liver sections were scored for periductal oedema based on an arbitrary score of 0 (no oedema) to 10 (highest amount of oedema observed). Only periportal areas were considered. Periductal oedema was defined as a prominent cuff with swollen connective tissue and an enlarged extracellular matrix around the bile duct.

### Assessment of Bacterial Translocation

Liver and spleen were harvested under sterile conditions after two- or seven-days post BDL. After weighing the tissue, sterilized PBS and stainless-steel beads were added into the samples, following by homogenizing the tissue by Tissuelyser (Quiagen) for 5 min at 30 Hz. The samples were then plated on lysogeny borth (LB) agar plates and cultured under aerobic conditions as described previously 16 (48 h at 37° C.). After the incubation, the bacterial colonies were counted manually, normalized to the weight of the organs to obtain the results as colony forming units. For the data visualization, the obtained CFU/g were normalized using a log transformation.

### Flow Cytometry

Antibodies used for fluorescence-activated cell sorting are listed in below. Livers were put in Roswell Park Memorial Institute (RPMI) 1640 Medium, supplemented with GlutaMAX and digested with collagenase D (Merck), 0.05% collagenase IV (Worthington Biochemical) and DNase I (Sigma-Aldrich) and Dispase for 25 min at 37° C. The digested liver-tissue was poured through a 100 μm cell strainer (BD Falcon) and washed in 50 ml RPMI at 4° C. After centrifugation at 300 g for 5 min, the supernatant was discarded and refilled with 30 ml RPMI. After repeating the centrifugation step at 300 g for 5 min discarding the supernatant, 5 ml Red Blood Cell (RBC) Lysis Buffer was added per sample and filtered through 40 μm cell strainer and incubated at room temperature. After incubation, the cell suspension was spun down and washed in 5 ml PBS (Gibco) containing 3% fetal bovine serum and 2 mM EDTA and Hepes (staining buffer). The isolated non-parenchymal cells were then incubated with purified anti-anti-CD16/CD32 and viability dye eFluor 506 (Thermo Fisher Scientific) diluted in PBS for 20 minutes at 4 C in the dark to block non-specific binding antibodies and exclude dead cells. Following the first staining and washing the cells, the samples were stained with primary antibodies (Table 1) diluted in the staining buffer for 20 minutes at 4 C in the dark. After the final washing step 300 g for 5 min, the samples were resuspended in the staining buffer and fixed by adding IC Fixations buffer (eBioscience). The fixed samples were assessed using the flow cytometer (BD LSR Fortessa; BD Pharmingen, Inc, San Diego, CA) using the corresponding BD FACS Diva software. Data analysis was performed using FlowJo software (Treestar, Inc, Ashland, OR).

### FACS Antibodies

| Fluorescence | Cell Marker | Clone | Company | Catalog no. |
|---|---|---|---|---|
| AF594 | CD45 | 30-F11 | Biolegend | 103144 |
| PE | CD3 | 17A2 | Biolegend | 100229 |
| APC CY7 | CD19 | 6D5 | Biolegend | 115530 |
| BV711 | CD8 | 53-6.7 | biolegend | 100748 |
| Pacific Blue | CD4 | RM4-5 | biolegend | 100531 |
| APC | CD11b | M1/70 | Biolegend | 101212 |
| PE-cy7 | LY6G | RB6-8C5 | Thermofisher | 25-5931-82 |
| Percp-CY5.5 | LY6-C | HK1.4 | eBioscience | 45-5932-82 |
| BUV395 | F4/80 | RUO | BD | 565614 |

### Total Bile Acid Measurement in Serum and Liver Tissue Samples

Total bile acid levels were measured using assay kits that were purchased from Crystal Chem (Cat. #80470). Bile, serum, and total liver tissue samples were processed according to the manufacturer instructions. Final absorbance levels were measured on a Tecan Tecan Spark® plate reader.

### Bile Acid Quantification Using LC-MS/MS

The method applied was described recently¹⁷. Briefly, for quantification of bile acids, 25 μL serum samples diluted 1:4 with water, and calibrators were subjected to protein precipitation by adding 900 μL of 2-propanol and a mixture of deuterated internal standards. Extraction was performed for 30 min at 4° C. with continuous shaking, followed by centrifuging 16000 g for 10 min. Supernatants were transferred to new tubes, evaporated to dryness and reconstituted with 100 μL methanol: water (1:1, v/v). For the extraction of liver samples, 900 μL of chloroform:methanol: water (1:3:1, v/v/v) and 100 μL internal standard mixture were added to a Precellys tube containing beads and 30±5 mg of liver tissue. Samples were homogenized with a Precellys tissue homogenizer, centrifuging 16000 g for 10 min at 20° C. The supernatant was transferred to a new tube and the procedure repeated by adding 800 μL of extraction solvent. After evaporation to dryness, samples were resuspended with 200 μL methanol: water (1:1, v/v). The injection volume was in both cases 3 μL. LC-MS/MS consisted of an Agilent 1290 UPLC coupled to an Agilent 6490 triple quadrupole mass spectrometer equipped with an electrospray ionization source (Agilent Technologies, Basel, Switzerland). Chromatographic separation of bile acids was achieved using a reversed-phase column (ACQUITY UPLC BEH C18, 1.7 mm, 2.1 um, 150 mm, Waters, Wexford, Ireland)¹⁸.

### ANIT Intoxication

α-Naphtylisothiocyanate (ANIT) intoxication protocols are derived from previous publications^{19,20}. In the acute intoxication model, ANIT (Sigma-Aldrich, St. Louis, Missouri, US) was dissolved in corn oil and administered orally at 60 mg/kg bodyweight. Same volumes of corn oil were given to control animals. A single- or double dose was given for the two- or ten days models. In the chronic injury model, custom produced chow containing 0.1% ANIT (Granovit, Lucens, Switzerland) was fed ad libitum. Custom chow containing same amounts of corn oil were fed to the control groups. Groups were fed the custom chows for a total of 4 weeks.

### Immunohistochemistry

Paraffin-embedded liver tissue was sectioned at a thickness of 6 μm for conventional imaging or 30 μm for confocal z-stack imaging. Slides were deparaffinized and hydrated in a xylol and ethanol series. Antigen retrieval was performed by heat-induced epitope retrieval, cooking the slides for 10 minutes at 95° C. in citrate buffer, pH 6.0 (Sigma-Aldrich, Cat. #C9999). Nonspecific antibody binding was blocked at room temperature for one hour using a protein blocking solution (Dako, Cat. #X0909). Antibodies were prepared in antibody diluent (Dako, Cat. #S3022) at the following dilutions. Primary antibodies: Cytokeratin 7 (Novus Biologicals, Cat. #NBP1-88080), 1:200. Secondary antibodies: polyclonal rabbit anti-goat immunoglobulins/HRP (Dako, Cat. #P0449). For the development of immunohistochemistry staining, streptavidin-peroxidase (BioConcept, Cat. #71-00-38) and DAB (Sigma-Aldrich, Cat. #D4293-50SET) were used.

Primary antibodies were incubated with gentle agitation inside a wet chamber overnight at 4° C. Slides were washed for 20 minutes in PBS-Tween-20 [0.5%] (Sigma-Aldrich, Cat. #P1379) and incubated in darkness for 90 minutes with the secondary antibodies and DAPI (Sigma-Aldrich, Cat. #D9542, diluted 1:2000). After a final wash in PBS-Tween 20 [0.5%], slides were mounted. Erythrocytes were quenched in 5% H₂O₂ for 10 minutes prior to the first antibody incubation, and the staining was developed after the secondary antibody application by incubation with streptavidin-peroxidase for 30 minutes and DAB for one minute.

### Western Blot

Total protein was extracted from liver tissue or cultured cells using RIPA lysis buffer and a TissueLyser (Qiagen, TissueLyser II). Lysates were centrifuged for 15 minutes at 20000 g, and the supernatant was aliquoted. Protein concentrations were quantified by Bradford assay (Bio-Rad, Cat. #5000006) and a microplate reader. Precast gels (Bio-Rad, Cat. #456-1094) were used to separate equalized amounts of protein per sample by SDS-PAGE, under reducing conditions. Proteins were then transferred on nitrocellulose membranes (Biorad, Cat. #170-4158). Membranes were blocked with 5% w/v nonfat dry milk in PBS for 1 hour at room temperature. Primary antibodies were diluted in the blocking medium and incubated overnight at 4° C. Primary antibodies: CLDN3, (Novus Biologicals, Cat. #NBP1-35668), 1:1000; Anti-β-Actin-Peroxidase, (Sigma-Aldrich, Cat. #A3854), 1:50000; Secondary antibodies: anti-rabbit-HRP (Dako, Cat. #P0448), 1:2000.

After primary antibody incubation, membranes were washed three times 5 minutes in PBS-Tween-20 [0.1%]. Secondary antibodies were diluted in with 5% w/v nonfat dry milk in PBS, and the membranes were incubated for one hour at room temperature, followed by three washing steps for 30 minutes in total. Enhanced chemiluminescence solution (Perkin Elmer, Cat. #NEL105001EA) was added for one minute to develop the signal. Films in combination with a developer (AGFA, CURIX 60) were used to visualize the bands. The correct band size was estimated with the help of a standard protein ladder (Biorad, Cat. #161-0374).

Human siRNA Candidate Screening

Candidate siRNAs targeting human claudin-3 mRNA were first identified bioinformatically (human reference sequence used: NM_001306.4). All possible siRNAs were created, and then scored for specificity, cross-reactivity, activity, and off-targets (Axolabs, Kulmbach, Germany). The best candidates were selected and synthetized, containing stabilizing modifications of 2′-fluoro- or 2′O-methyl modifications, and phosphorothioate linkers at the indicated positions (see table 6). The synthetized candidate siRNAs were then transfected into the human PLC liver cell hepatoma cell line (ATCC Cat. No CRL-8024) using lipofectamine-3000. siRNAs were used at a final concentration of 50 nM. Two days after transfection, RNA was isolated from the cell cultures, and the levels of remaining human claudin-3 mRNA were quantified using RT-qPCR following the protocols below. 34 siRNAs with a knockdown efficacy of 50% or higher were selected for further testing (table 6). Table 7 displays the same identified human claudin-3 siRNA sequences, without their chemically stabilizing modifications.

RT-qPCR mRNA Expression Analysis

RNA from snap frozen tissue or cell cultures was extracted using NucleoZOL (Macherey-Nagel, Cat. #740404.200). cDNA was made from either 500 μg of tissue RNA using the Omniscript reverse transcriptase kit (Qiagen, Cat. #205113). Real-time qPCRs have been performed on an QuantStudio™ 7 Flex thermocycler (Applied Biosystems, Foster City, CA) using either SYBR-green or Taqman based assays, according to the manufacturers instructions. Primer sequences are listed below.

### Primers Sequences (m; Mouse, F; Forward, R; Reverse)

| Primer (Taqman) | Target | Catalogue Number | Manufacturer |
|---|---|---|---|
| Hs99999905_m1 | Human Gapdh | #4351370 | Thermo Fisher |
| Hs00265816_s1 | Human Claudin-3 | #4331182 | Thermo Fisher |
| Primer (SYBR) | Sequence 5′-3′ | Source | SEQ ID NO: |
| mEef1a1_F | CGTTCTTTTTCGCAACGGGT | NCBI Primer-BLAST | 7 |
| mEef1a1_R | TTGCCGGAATCTACGTGTCC | NCBI Primer-BLAST | 8 |
| mCldn3_F | GCACCCACCAAGATCCTCTA | 21 | 9 |
| mCldn3_R | TCGTCTGTCACCATCTGGAA | 21 | 10 |
| mCyp7a1_F | AGCAACTAAACAACCTGCCAGTACTA | 22 | 11 |
| mCyp7a1_R | GTCCGGATATTCAAGGATGCA | 22 | 12 |
| mCyp7b1_F | AATTGGACAGCTTGGTCTGC | 22 | 13 |
| mCyp7b1_R | TTCTCGGATGATGCTGGAGT | 22 | 14 |
| mCyp27a1_F | GTGGACAACCTCCTTTGGGA | 22 | 15 |
| mCyp27a1_R | TTGCTCTCCTTGTGCGATGAA | 22 | 16 |
| mGsta1_F | GGCTTTCAAGATTCAGTGAA | 23 | 17 |
| mGsta1_R | TAGCCAGGATCAACAATTGCT | 23 | 18 |
| mCyp4a14_F | CCCAAAGGTATCACAGCCACAA | 22 | 19 |
| mCyp4a14_R | CAGCAATTCAAAGCGGAGCAG | 22 | 20 |
| mOst1b_F | AGATGCGGCTCCTTGGAATTA | 24 | 21 |
| mOst1b_R | TGGCTGCTTCTTTCGATTTCTG | 24 | 22 |
| mOatp1a1_F | GCCAACGCAAGATCCAACAGAGTG | 25 | 23 |
| mOatp1a1_R | TCGGGCCAACAATCTTCCCCAT | 25 | 24 |
| mOatp1b2_F | TGGAAGGCATAGGGTAGGCGGT | 25 | 25 |
| mOatp1b2_R | TGGGCAGCTTTGCTTGGATGCT | 25 | 26 |
| mNtcp_F | AATCCAAGCTGCAGACGCACC | 25 | 27 |
| mNtcp_R | GCATCTTCTGTTGCAGCAGCCTT | 25 | 28 |
| mMDR2_F | TGGCCGATGTGTGTGAGTACA | 26 | 29 |
| mMDR2_R | TGCCTGGCACCAAAAGGT | 26 | 30 |
| mMrp3_F | GGGCTGCCTTGCCCTGCTAC | 25 | 31 |
| mMrp3_R | CCGAGGGCCGTCTTGAGCCT | 25 | 32 |

### Antibody Production and Sequence

The recombinant mouse IgG2A Kappa anti Claudin 3 antibody (UB-VS003) was generated using Expi293 cells. The antibody consists of a heavy chain (SEQ ID NO: 170) and a light chain (SEQ ID NO: 169) as displayed below.

Upon transfection of the Expi293 cells with the expression vector, the cells efficiently produced and secreted the recombinant antibody. The cell supernatant was then purified using protein A resin (Repligen, #CA-PRI-0005).

Light chain (SEQ ID NO: 169):
DIVMSQSPSSLAVSVGEKVTMSCKSSPSLLYSNNQKNYLAWYQQKPGQSPKLLIYWA
STRESGVPDRFTGSGSGTDFTLTISSVKAEDLAVYYCQQYYSFPYTFGGGTKLEIKRAD
AAPTVSIFPPSSEQLTSGGASVVCFLNNFYPKDINVKWKIDGSERQNGVLNSWTDQDS
KDSTYSMSSTLTLTKDEYERHNSYTCEATHKTSTSPIVKSFNRNEC
Heavy chain (SEQ ID NO: 170):
EVQLQQSGPELVKPGGSMKISCKASGYSFTGYTMNWVKQSHGKNLEWIGLINPYNDG
TNYNQKFKGKATLTVDKSSSTAYMELLSLTSEDSAVYYCSKKGGGYGGTWFAYWG
QGTLVTVSAAKTTAPSVYPLAPVCGDTTGSSVTLGCLVKGYFPEPVTLTWNSGSLSSG
VHTFPAVLQSDLYTLSSSVTVTSSTWPSQSITCNVAHPASSTKVDKKIEPRGPTIKPCPP
CKCPAPNLLGGPSVFIFPPKIKDVLMISLSPIVTCVVVDVSEDDPDVQISWFVNNVEVH
TAQTQTHREDYNSTLRVVSALPIQHQDWMSGKEFKCKVNNKDLPAPIERTISKPKGSV
RAPQVYVLPPPEEEMTKKQVTLTCMVTDFMPEDIYVEWTNNGKTELNYKNTEPVLD
SDGSYFMYSKLRVEKKNWVERNSYSCSVVHEGLHNHHTTKSFSRTPGK

### Primary Cells and Cell Line Culture Methods

Liver samples were obtained from patients undergoing liver resection at University Hospitals in Basel and Bern, Switzerland. Human hepatocytes and mouse hepatocytes from C57BL/6 and C57BL/6 Cldn3^{-/-} mice were isolated from the liver specimens following a two-step enzymatic perfusion protocol (doi: 10.1172/JCI115207). The viability of the isolated hepatocytes was determined by trypan blue exclusion, and only preparations of over 90% viability were used. The hepatocytes were seeded onto tissue culture plastic coated with rat tail collagen in Dulbecco's modified Eagle medium containing 10% fetal bovine serum, left to attach for 1 to 2 hours, and then washed twice with phosphate-buffered saline (PBS) to remove any remaining non-viable cells from the culture. The hepatocytes were cultured in arginine-free Williams E medium supplemented with insulin (0.015 IU/mL), hydrocortisone (5 μmol/L), penicillin (100 IU/mL), streptomycin (100 μg/mL), glutamine (2 mmol/L), and ornithine (0.4 mmol/L) for 24 hours before use.

SNU-449 cells (ATCC #2234) were grown in RPMI 1640 medium supplemented with 10% fetal bovine serum, 100 U/mL penicillin, 100 mg/mL streptomycin. Hepa1-6 (ATCC #CRL-1830) were grown in DMEM supplemented with 10% fetal bovine serum, 100 U/mL penicillin, 100 mg/mL streptomycin.

### Flow Cytometry for Human Claudin-3 Antibody Tests

Binding capacity of the Cldn3 antibody was accessed by serial dilution of the stock solution of the anti-Claudin 3 antibody (0.5 mg/ml) in facs buffer exposed to 5.105 human hepatocyte or SNU449 cells for 30 min at 4c. The cells are then rinsed with PBS and exposed to a cyanine5 conjugated secondary anti mouse IgG (Life technologies #A10524) at 1:200 for 30 min at 4c in the darc, before being rinsed with PBS. Finally, cell data were acquired on a SORP LSRII (BD Pharmingen Inc., San Diego, CA). Flow cytometric analysis was done using FlowJo software (Treestar, Inc., Ashland, OR).

### Binding Capacity Assessment of Cldn3 Antibody

To determine the binding capacity of the anti-Claudin 3 antibody, a serial dilution of the stock solution (0.5 mg/ml) was prepared in FACS buffer. The diluted antibody solutions were then exposed to 5.105 human hepatocyte or SNU449 cells for 30 minutes at 4° C.

Following the incubation period, the cells were rinsed with PBS to remove unbound antibody. Subsequently, a secondary FITC-anti-mouse IgG (ref) was applied to the cells at a dilution of 1:200. The secondary antibody was allowed to bind for 30 minutes at 4° C. in the dark. Afterward, the cells were washed with PBS to eliminate any excess secondary antibody.

Cellular data acquisition was performed using a SORP LSRII flow cytometer (BD Pharmingen Inc., San Diego, CA). The acquired data were then analyzed using FlowJo software (Treestar, Inc., Ashland, OR) for flow cytometric analysis.

### GalNAc-Anti Cldn3 Antibody Functionalization

The antibody was first buffer exchanged into PBS using a 7K Zeba size-exclusion column, which effectively removed any unwanted buffer components and ensured compatibility with subsequent reactions.

To functionalize the antibody, it was reacted with 25 equivalents of DBCO-PEG4-NHS (BroadPharm #BP-22288). The antibody solution in PBS was incubated overnight at room temperature to allow for efficient conjugation between the antibody and the DBCO-PEG4-NHS.

Following the conjugation reaction, the functionalized antibody was purified using aPBS equilibrated 7K Zeba size-exclusion column. This purification step helped to separate the conjugated antibody from any unreacted DBCO-PEG4-NHS and other impurities, resulting in a purified functionalized antibody preparation.

Next, the purified functionalized antibody was reacted with 100 equivalents of Trebler GalNAc-Azide (primetech #0079). This reaction facilitated the attachment of Galactose N acetyl (GalNAc) moieties to the functionalized antibody, enabling targeted delivery to GalNAc receptors.

After the conjugation reaction with GalNAc-Azide, the antibody was once again purified, this time using a PBS equilibrated 40K Zeba size-exclusion column. This purification step further separated the conjugated antibody from any unreacted GalNAc-Azide and other remaining impurities, yielding a purified and functionalized antibody ready for downstream applications.

### Cldn3 KD Using GalNAc-Anti Cldn3 Antibody

Human hepatocytes in 12 well plate were treated with varying concentrations of the GalNAc-anti Cldn3 antibody. Specifically, cells were treated with 0, 1, 10, and 100 nM concentrations of the antibody for a duration of 24 hours. Following the 24-hour treatment period, proteins were extracted from the treated cells using 50 μl of RIPA lysis buffer.

The extracted proteins were quantified using the Bio-Rad Protein Assay System (Bio-Rad Laboratory, Melville, NY).

### Western Blot to Test Human Claudin-3 Inhibiting Antibody Efficacy

Protein lysates were boiled in Laemmli buffer, run on BIO-RAD Mini-Protean TGX™ (10-20% Ready Gel Tris-HCl Gel System, 12-well comb #456-1095 and 10-well comb #456-1094) for 90 min at 120 Volt, and transferred on BIO-RAD Trans-Blot Turbo Mini or Midi PVDF membranes (Mini PVDF Transfer Packs #170-4156 and Midi PVDF Transfer Packs #170-4159) by semi dry Transfer (Trans-Blot Turbo Transfer System BIO-RAD). Proteins were detected using the following primary antibodies: Claudin 3 (1:300) (Novus Biologicals, Cat. #NBP1-35668) β-actin HRP-conjugated (1:5000) (Sigma-Aldrich, #2228, RRID: AB_476697). Antibodies were diluted in 5% milk and incubation was done overnight at 4° C. Following secondary antibodies were used: HRP-conjugated anti-rabbit (1:2000) (Dako, #P0448, RRID: AB_2617138). Membranes were then washed, and protein expression was analysed by chemiluminescence (Western Lightning Plus-ECL Perkin Elmer), using Fusion-FX (Vilber).

### Bile Autofluorescence Measurement

Bile obtained from gallbladders was diluted 1:50 with water. Autofluorescence was measured at excitation 489 nm/emission 534 nm, using a Tecan Spark® plate reader.

### Bulk RNA Sequencing of Tissue Samples

Total RNA was extracted from the liver with NucleoZOL, quantified by a bioanalyzer. The setting of the sequencing run included TruSeq Stranded mRNA, with paired-read ends. Read length was set to 50, and the multiplex level was 1.

### RNA-seq Alignment

fastq files were aligned to the mouse reference genome mm 10 with hisat2 and transformed into bam files with samTOOLS. The read count matrix was produced from the bam files with the featurecounts function of the R package Rsubread.

### Dimensionality Reduction

Principal Component Analysis: Read count matrix was normalized to Reads Per Million (RPMs), and log transformed, f(x)=(1+log(x)). Principal Components were computed with the R function prcomp. The data was displayed with ggplot2. Heatmap: Read count matrix was normalized to RPMs. When comparing gene expression in different conditions we normalized every gene from 0 to 1 f(x_i)=(x_i−min(x_i))/(max(x_i)−min(x_i). where max(x_i) is the max value of gene i in all conditions (analogous for min(x_i). When comparing gene expression in cell types, we normalized every cell type from 0 to 1 f(x_i)=(x_i−min(x_i))/(max(x_i)−min(x_i). where max(x_i) is the max value of cell i in all genes.

### RNA-seq Differential Expression

Differentially expressed genes were computed with R package DESeq2. Genes with an adjusted by False Discovery Rate p-value below 0.05 were considered statistically significant for further analysis.

### Enrichment Analysis

To determine the pathways to which genes were associated we used Metascape. Statistical significant genes. When more than 2000 were present in a list we took the top 2000 sorted by Fold Change.

GalNAc siRNA Constructs and GalNAc siRNA Injections

The following siRNA sequences were prepared (synthesis nomenclature: n=2′OMe-RNA, Nf=2′-Fluoro-RNA, s=phosphorothioate):

Claudin-3 targeting siRNA1:
• sense strand sequence (5′-3′):
(SEQ ID NO: 1)
csusAfcCfaGfcAfgUfcGfaUfgAfaAf
• antisense strand sequence (5′-3′):
(SEQ ID NO: 5)
UfsUfsuCfaUfcGfaCfuGfcUfgGfuAfgsusu
Claudin-3 targeting siRNA2:
• sense strand sequence (5′-3′):
(SEQ ID NO: 2)
gsasAfaCfgGfgCfcAfuUfuCfaUfaAf
• antisense strand sequence (5′-3′):
(SEQ ID NO: 6)
UfsUfsaUfgAfaAfuGfgCfcCfgUfuUfcsusu
AHSA1 control siRNA
• sense strand sequence (5′-3′):
(SEQ ID NO: 3)
uscsUfcGfuGfgCfcUfuAfaUfgAfaAf

The above indicated siRNAs were synthetized by Axolab, Kulmbach, Germany. All siRNAs were created from mouse Cldn3 mRNA sequence (NM_009902.4). The bases within the siRNAs were modified for more chemical stability using 2′Fluoro- and/or 2′O-Methyl residues and phosphorothioate linkages. Claudin-3 targeting siRNA1 (sense strand sequence), Claudin-3 targeting siRNA2 (sense strand sequence) and AHSA1 control siRNA (sense strand sequence) were conjugated with a triantennary GalNAc cluster (CPG, Primetech). siRNAs were HPLC-purified and lyophilized. For in vivo use, GalNAc siRNAs were diluted with saline solution. The siRNAs were injected subcutaneously at a concentration of 10 mg/KG bodyweight. Mice were pre-treated with the siRNAs for two- or four days prior to the experiment.

### Results

### Claudin-3 Knockout Leads to Impaired Bile Acid Metabolism, Dilution of Bile, a Higher Bile Flow Rate and Impaired Uptake of the Tracer FITC-Dextran.

By analysing our published RNAseq dataset comparing Cldn3^{+/+} and Cldn3^{-/-} mice²⁷, we found out that bile acid- and bile salt metabolism are among the top gene pathways that are lower expressed in absence of claudin-3 (FIG. 1A). The differential gene expression analysis revealed genes like Cyp27a1, that have a key function in BA synthesis, to be less expressed (FIG. 1B). We confirmed this result for Cyp27a1 and showed also lower expression of Cyp7b1 in Cldn3^{+/+} livers using RT-qPCR (FIG. 1C). We wondered whether the suppressed BA synthesis would affect the amount of total bile acids (TBA) in bile. Whereas the bile TBA levels of Cldn3^{+/+} mice were on average at 4194 μmol/L, Cldn3^{-/-} bile contained only 3932 μmol/L (P<0.01) (FIG. 1D). It has been described that bile has autofluorescent properties²⁸ that can be mostly ascribed to the abundance of bilirubin. We measured bile autofluorescence at an excitation of 489 nm and an emission of 534 nm. Cldn3^{+/+} bile had 499.7 relative fluorescence units (RFU)/μl bile/g liver, whereas Cldn3^{-/-} bile autofluorescence was surprisingly drastically lower with only 14.4 RFU/μl bile/g liver (P=0.0079) (FIG. 1E), indicating that the bile is diluted for bilirubin.

Next we aimed to determined the bile flow rate and the uptake- and paracellular permeability of the blood biliary barrier in Cldn3^{-/-} mice. We used an experimental setup that has been described previously¹⁴. In brief, a common bile duct ligation (BDL) was performed, and the gallbladder was canulated (FIG. 1F). Bile was collected over a twenty-minute period to determine the bile flow (FIG. 1G). We found that the bile flow was much faster with 3.7 μl/min/g liver in Cldn3^{-/-} mice, compared to only 0.8 μl/min/g liver in Cldn3^{+/+} mice (P=0.0036) (FIG. 1G).

In the same setup, we tested the paracellular barrier by injecting the fluorescent tracer FITC-Dextran intravenously (IV) and collecting bile in 3-minute intervals afterwards (FIG. 1H). We found that FITC-Dextran gets increasingly uptaken- and secreted into bile starting from 3 minutes on, however the abundance of FITC-Dextran was strikingly lower at any time in Cldn3^{-/-} mice (FIG. 1H). This shows that the hepatocyte paracellular barrier is not majorly impaired for FITC dextran passage

Next, we proved our hypothesis of impaired FITC dextran (40 kDa) uptake in Cldn3^{-/-} mice further. We performed a BDL and injected FITC dextran (40 kDa, 6.25 mg/ml, 300 μl volume) via the tail vein. On the anaesthetized and mice with laparotomy, we continuously imaged FITC dextran fluorescence for 45 minutes using the IVIS® Spectrum In Vivo Imaging System (PerkinElmer). For analyzing FITC dextran intensity in the liver, we selected regions of interest of the same area and determined the FITC signal using the device software. At twenty minutes post injection, a lower intensity of FITC signal can be observed in the Cldn3^{-/-} liver (FIG. 1I). Quantification of the FITC signal over time showed that FITC dextran is rapidly uptaken by Cldn3^{+/+} livers but but by Cldn3^{-/-} livers (FIG. 1J). Using the same experimental setup, we harvested livers at 20 minutes post injection and prepared 25 μm thick cryosections. The cryostection were stained with Dapi for 15 minutes and then confocal Z-stack imaging was performed (LSM 710; Zeiss, 20× objective, Oberkochen, Germany). Quantification of FITC fluorescence in the entire Z-Stacks showed that Cldn3^{-/-} livers took up significantly less FITC dextran at 20 minutes post injection (P=0.0049, n=3/4, students t test).

Collectively, our data shows that loss of claudin-3 impairs BA synthesis and leads to bile that is diluted in total bile acids and bilirubin. The bile flow of Cldn3^{-/-} mice is quicker, and the interhepatocyte paracellular permeability was not impaired for FITC dextran passage. However, Cldn3^{-/-} livers had a reduced uptake- and biliary secretion of FITC-Dextran (40 kDa).

Absence of Claudin-3 Protects from Obstructive Cholestatic Liver Injury

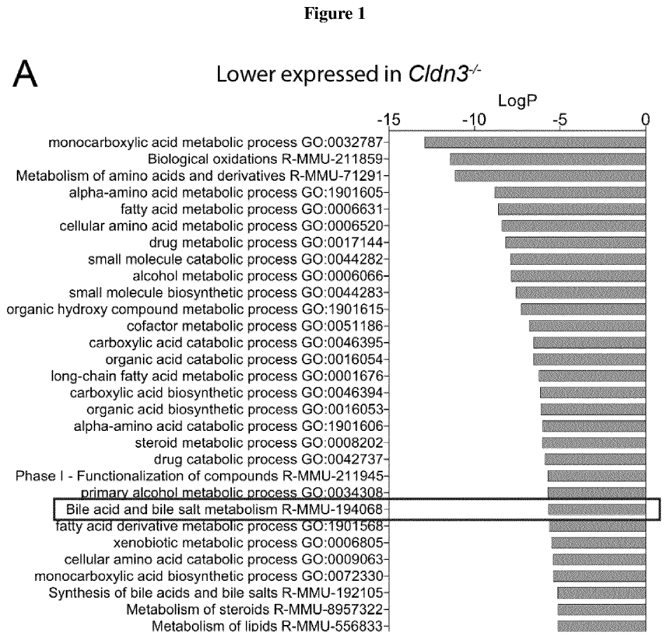

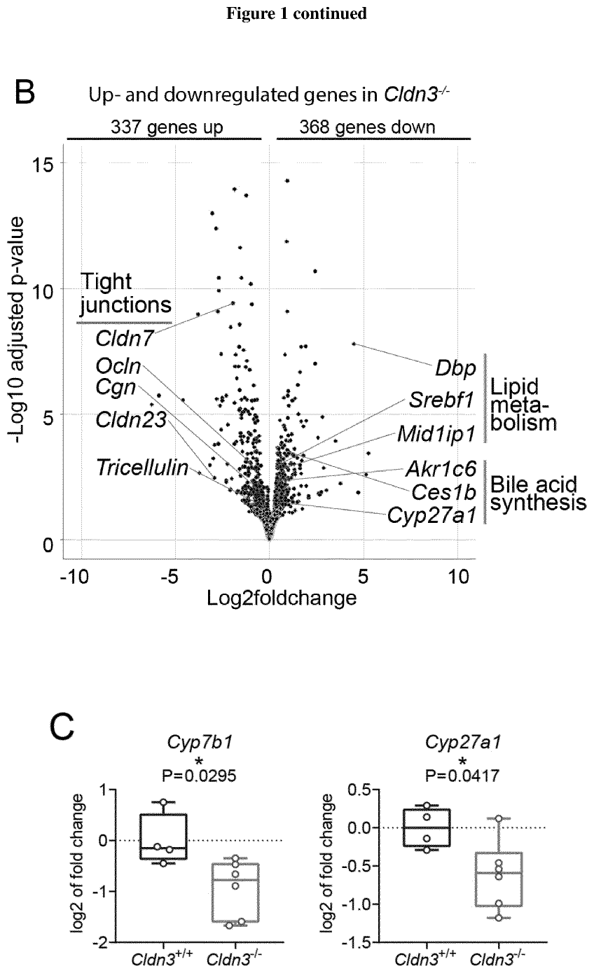

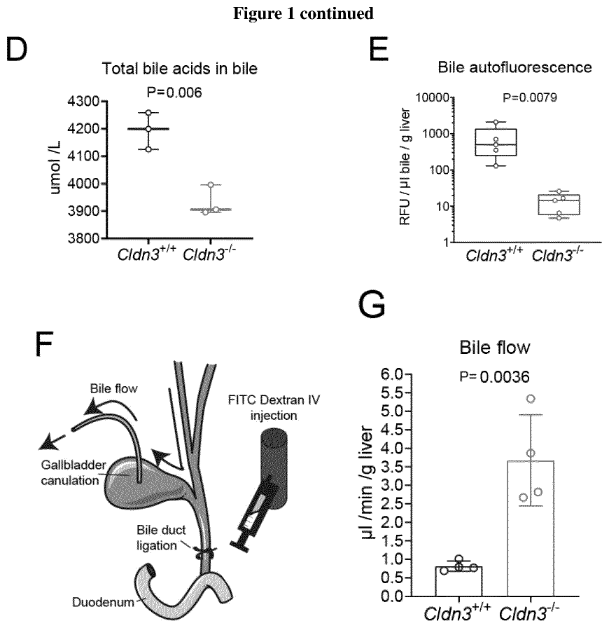

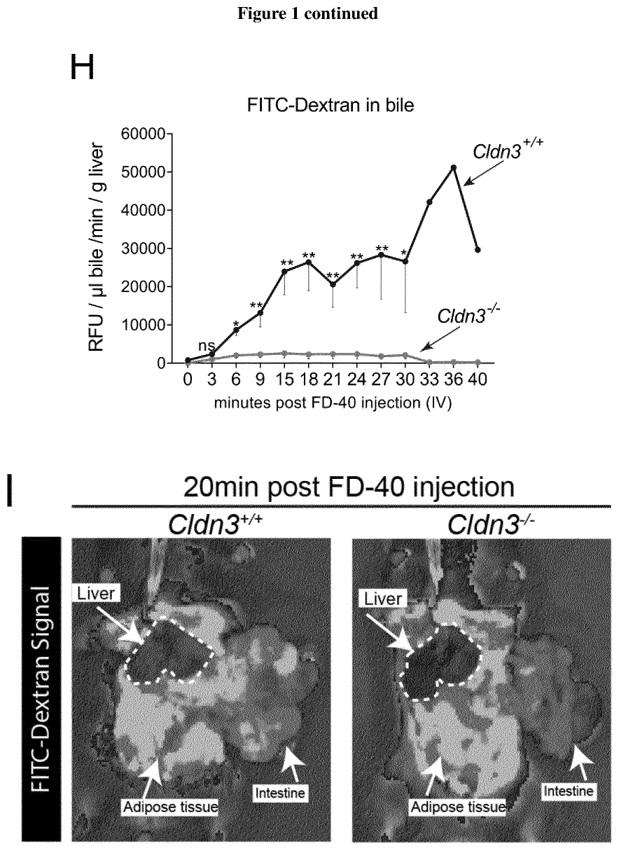

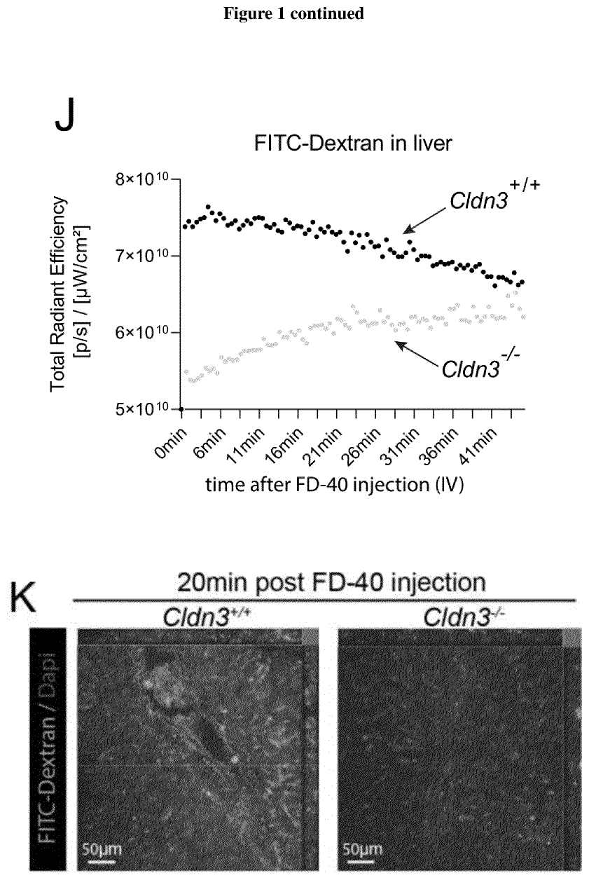

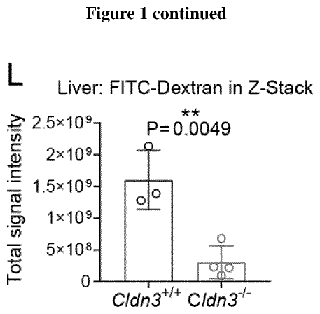

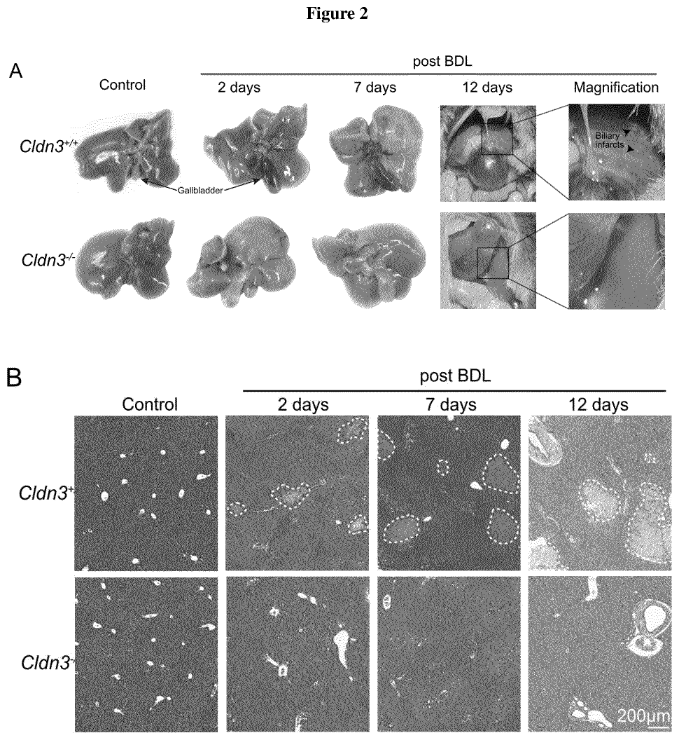

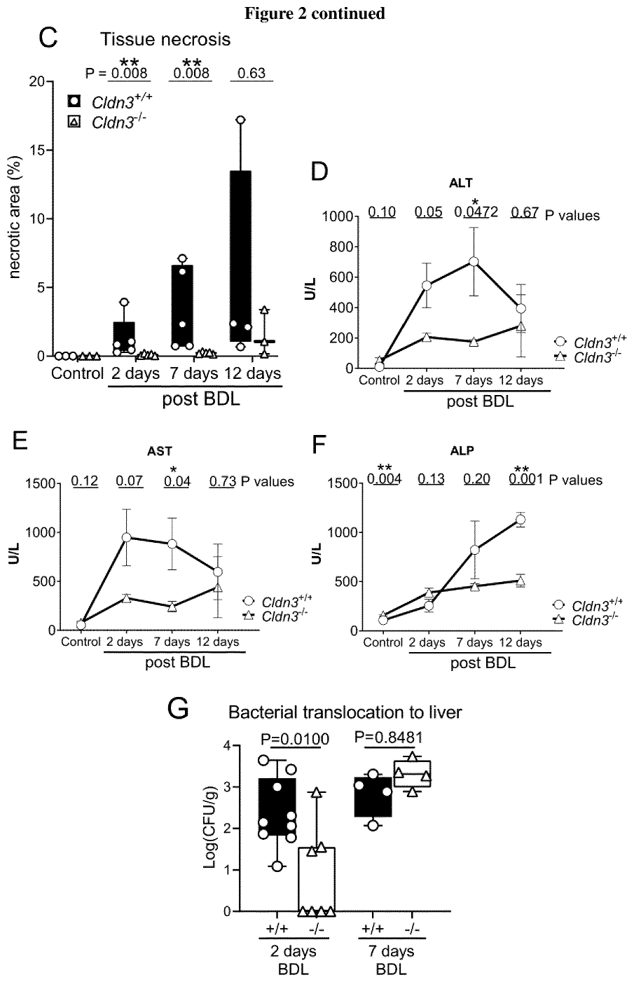

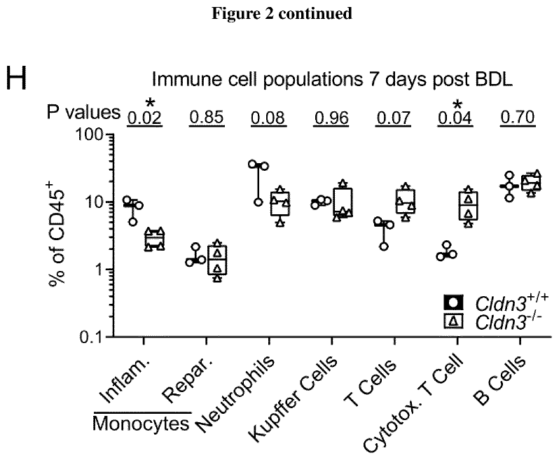

As bile acids are regarded as the main cause for pathobiology of cholestatic liver diseases²⁹, we next question the outcome of the Cldn3^{-/-} bile dilution for cholestasis. We first applied an extrahepatic obstructive cholestasis model. Cldn3^{+/+} and Cldn3^{-/-} mice were subjected to BDL for a duration of either two-, seven-, or twelve days. Macroscopic photos showed as expected no difference in the control groups. However, following BDL we observed striking differences in the liver appearance. Wildtype animals expressed the expected BDL phenotype, including dark green coloured bile and visible necrotic patches in liver tissue (FIG. 2A). These features were absent in the Cldn3^{-/-} group, indicating a different composition of the bile and significantly ameliorated necrosis.

The histological analysis of the liver tissue showed no difference among control groups, which had a normal liver morphology (FIG. 2B). Following BDL, wildtype animals developed tissue necrosis as a typical features of obstructive liver injury (FIGS. 2B and C), which gained in severity as the cholestasis progressed. Strikingly, mice lacking claudin-3 developed almost no necrosis (FIGS. 2B and C). Accordingly, the levels of clinical liver injury markers were significantly lower in absence of claudin-3. At seven days post BDL, alanine aminotransferase (ALT) levels were on average at 702 U/L in Cldn3^{+/+} mice compared to only 176 U/L in Cldn3^{-/-} mice (FIG. 2D). Aspartate transaminase (AST) levels were at 882 U/L in Cldn3^{+/+} mice and only at 242 U/L in Cldn3^{-/-} mice at 7 days post BDL (FIG. 2E). Alkaline phosphatase levels (ALP) significantly differed after 12 days post BDL with on average 822.5 U/L in Cldn3^{+/+} mice and 452.0 U/L in Cldn3^{-/-} mice (FIG. 2F).

It was previously described that BDL leads to translocation of bacteria to the liver, being an indicator of tissue injury¹⁶. We plated homogenates of livers after BDL and counted the number of colony forming units (CFU). The quantification showed that the number of bacteria that translocated to the liver was significantly lower in Cldn3^{-/-} livers at 2 days post BDL compared to wildtype livers (P=0.01) (FIG. 2G). At 7 days post BDL the amount of CFU was similar among groups.

As last indicator of inflammation and injury, we quantified the frequency of immune cells after 7 days post BDL using fluorescence-activated cell sorting (FACS). We found that the frequency of inflammatory monocytes was significantly lower; whereas cytoprotective and inflammation resolving T cells were higher in frequency in cholestatic Cldn3^{-/-} livers (FIG. 2H). There was a trend for lower neutrophil frequency in Cldn3^{-/-} livers, too. The other analysed populations did not differ, including B cells and Kupffer cells.

Collectively, our results show that loss of claudin-3 leads to a striking amelioration of inflammation and necrosis and protects the murine liver from injury in this obstructive cholestasis model.

### Reduced Bile Acid Concentration in Liver and Gallbladder Bile Following BDL

We next explored the mechanism causing of to the protective effect of claudin-3 loss further. The bile acid composition is mildly changed in Cldn3^{-/-} mice^{27,30} under normal conditions. We hypothesized that a significant reduction in bile acid levels could be explanatory for the injury amelioration. We therefor analysed the concentration and composition of hepatic bile acids in the BDL model.

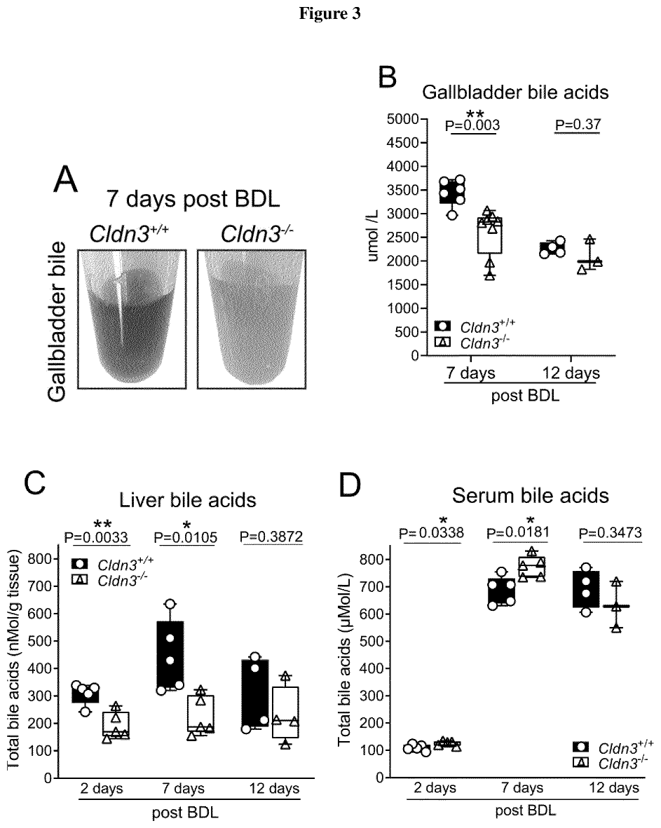

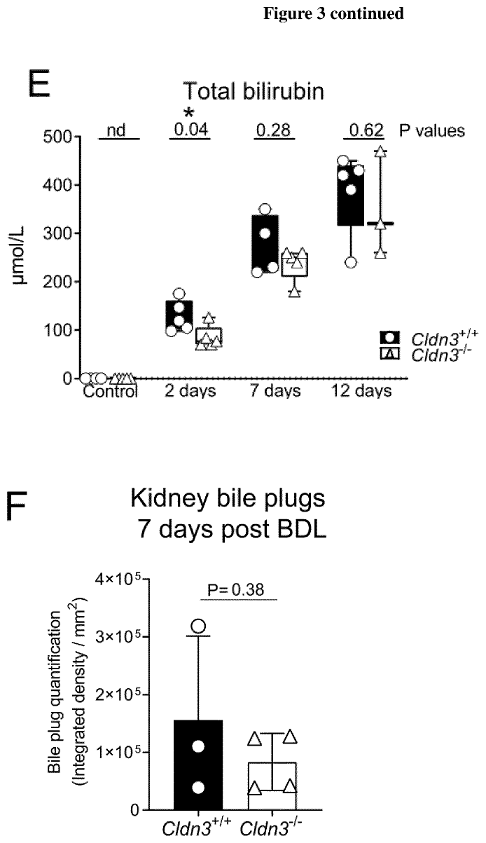

Bile retrieved from Cldn3^{+/+} mice at 7 days post BDL from the gallbladder had a yellow- or dark green colour whereas Cldn3^{-/-} bile was yellow (FIG. 3A, Cldn3^{-/-} bile appears darker in the grayscale images). TBA levels in gallbladder bile collected 7 days post BDL were 3435 μmol/L in Cldn3^{+/+} and only 2611 μmol/L in Cldn3^{-/-} (P<0.01) (FIG. 3B). Liver TBA at 2 days post BDL was on average 308.3 nmol/g in Cldn3^{+/+} and only 191.8 nmol/g in Cldn3^{-/-} liver (p<0.01) (FIG. 3C). In conjunction with the higher amount of liver injury (FIG. 2), hepatic TBA levels raised after 7 days, with an average of 447.2 nmol/g in Cldn3^{+/+} mice and significantly less in Cldn3^{-/-} mice with 226.2 nmol/g (p<0.05) (FIG. 3C).

Interestingly, the difference in TBA levels was inverted when testing the serum (FIG. 3D). Two days post BDL, average serum TBA levels were 109.1 μmol/L in Cldn3^{+/+} mice and 125.6 μmol/L in Cldn3^{-/-} (p<0.05) (FIG. 3D). Serum TBA levels increased at 7 days and the difference among groups became larger. After 7 days, Cldn3^{+/+} serum contained 689.2 μmol/L, while Cldn3^{-/-} serum contained significantly more TBA with 774.1 μmol/L (p<0.05) (FIG. 3D). Of note, there were no significant differences in either hepatic, serum or bile TBA at the 12-day time point (FIG. 3B-D). We analysed the serum further by checking bilirubin levels, which were lower in Cldn3^{-/-} mice at 2 days post BDL, but at no other time point (FIG. 3E).

We also checked for a potential negative effect of the slightly increased serum bile acid levels on kidneys of Cldn3^{-/-} mice. We quantified the number of bile plugs (also referred to as “bile casts”) in the kidneys and could not find differences between Cldn3^{+/+} and Cldn3^{-/-} mice at seven days post BDL (FIG. 3F).

Altogether, the data demonstrate that claudin-3 deficient animals have diluted bile also after induction of cholestasis. We found significantly lower levels of total bile acids in the liver and gallbladder, but higher levels in the serum. This relief of the hepato-biliary system from bile acid related cytotoxicity could potentially explain the observed amelioration in liver injury.

Claudin-3 Deletion does not Majorly Alter of the Composition of the Bile Acid Pool Under Normal- or Cholestatic Conditions

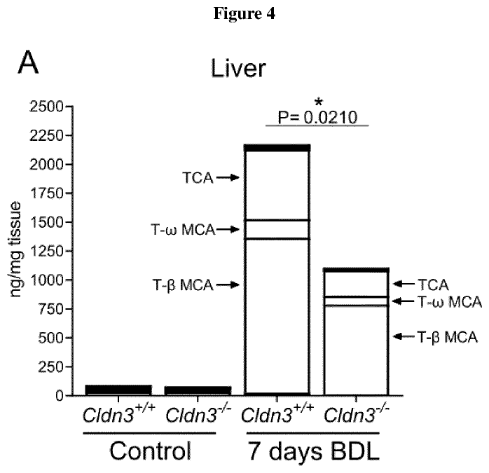

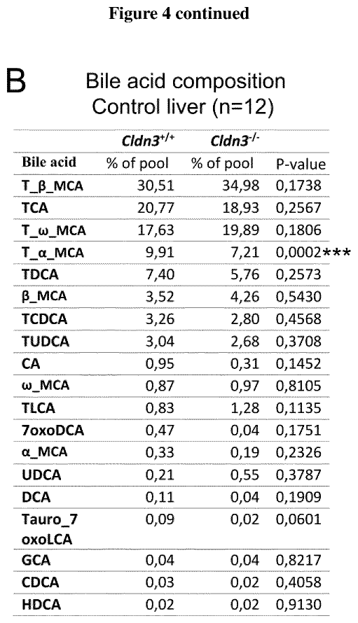

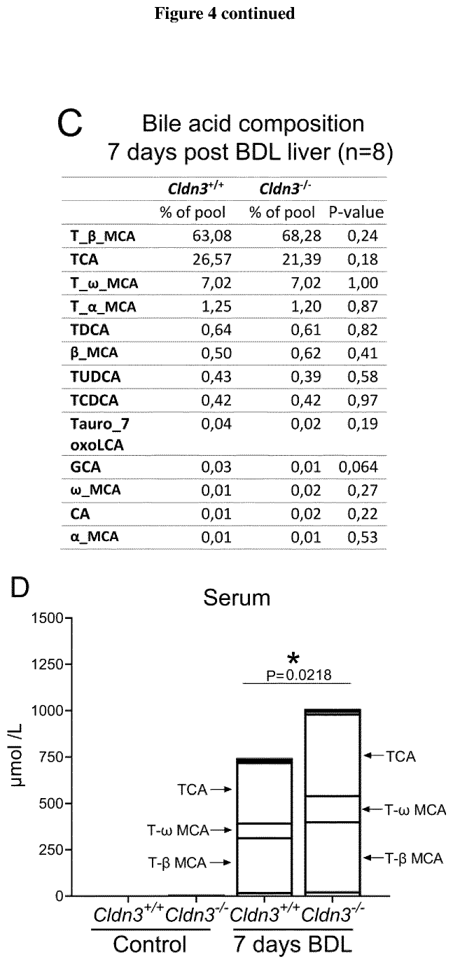

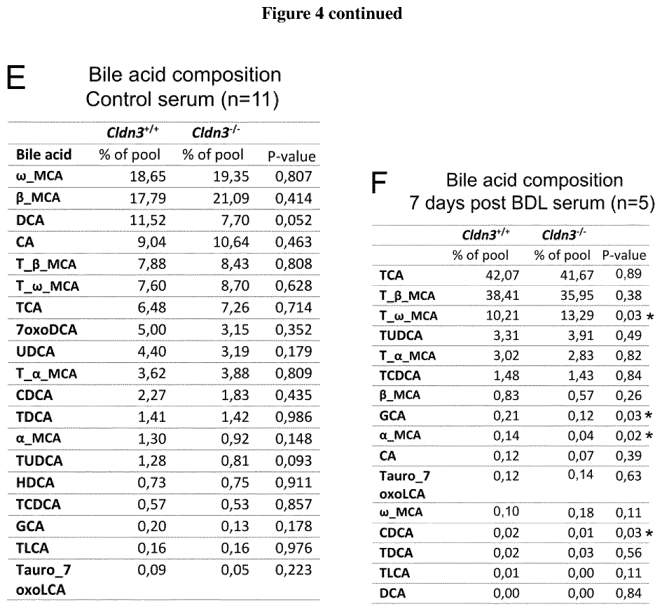

During cholestasis, increased hydrophobicity of bile can be important contributor to tissue damage, which depends on the composition of bile with individual bile acid (BA) subtypes. We questioned whether claudin-3 deficient mice have a different BA pool with—or without cholestasis. Therefore, we determined the concentration of individual BA types in the liver—and serum in control animals and at seven days post BDL, using Liquid chromatography-mass spectrometry (LC-MS/MS) (FIG. 4 and table 1). We observed that conjugated muricholic acids T-αMCA, T-βMCA, T-ωMCA and tauro-cholic acid (TCA) were by far the most abundant bile acid species in the liver bile acid pool (FIG. 4A-C and table 1). Reporting the bile acid amount as nanogram/milligram liver tissue showed that hepatic bile acid levels raise dramatically after 7 days BDL (FIG. 4A). The LC-MS/MS analysis further confirmed our previous observation of reduced liver bile acid levels in Cldn3^{-/-} livers at seven days post BDL (FIGS. 3C and 4A).

We next calculated the percentages of bile acid subtypes from the whole liver bile acid pool. Comparing the bile acid subtypes of Cldn3^{+/+} to Cldn3^{-/-}, there were only small differences, such as a slightly lower percentage of T-αMCA in control liver tissue of Cldn3^{-/-} mice (FIG. 4B and table 1). There where no differences in the liver bile acid pool composition of Cldn3+ and Cldn3^{-/-} after seven days post BDL. The data showed that the diversity of the whole hepatic bile acid pool was reduced in favor of the main bile acid subtypes, when comparing control to 7 days post BDL (FIGS. 4B and C and table 1).

We next analysed the serum bile acids. Quantification of bile acids in μmol/L showed significantly higher levels in Cldn3^{-/-} serum at seven days post BDL (FIG. 4D), confirming our previous results (FIG. 3D). Calculation of the percentages of bile acid subtypes from the entire bile acid pool revealed no differences comparing of Cldn3^{+/+} to Cldn3^{-/-} control serum (FIG. 4E and table 1). But at seven days post BDL, we observed minor differences in Cldn3−/− serum, such as slightly higher percentage in T-ωMCA and slightly lower percentages in GCA, αMCA and CDCA (FIG. 4F and table 1). The diversity of the serum bile composition was decreased after seven days BDL when compared to control (FIGS. 4E and F). We observed that the diversity of the serum bile composition changed in favor of TCA and T-βMCA, as previously seen in liver tissue (FIGS. 4 B and C).

Taken together, the analysis of liver- and blood bile acids confirmed the previous findings of reduced hepatic bile acid- and raised blood bile acid levels in Cldn3^{-/-} mice. We could not find major differences in the bile acid pool composition when comparing Cldn3^{+/+} to Cldn3^{-/-} liver or serum.

TABLE 1
Bile acid pool composition in Cldn3^{+/+} and Cldn3^{-/-} livers, pre (control)- and post BDL.

| Control liver bile acid pool | | | | Control serum bile acid pool | | | |
|---|---|---|---|---|---|---|---|
| | Cldn3^{+/+} | Cldn3^{-/-} | | | Cldn3^{+/+} | Cldn3^{-/-} | |
| Bile acid | % of pool | % of pool | t test | Bile acid | % of pool | % of pool | t test |
| T_β_MCA | 30.51 | 34.98 | 0.1738 | ω_MCA | 18.65 | 19.35 | 0.807 |
| TCA | 20.77 | 18.93 | 0.2567 | β_MCA | 17.79 | 21.09 | 0.414 |
| T_ω_MCA | 17.63 | 19.89 | 0.1806 | DCA | 11.52 | 7.70 | 0.052 |
| T_α_MCA | 9.91 | 7.21 | 0.0002 | CA | 9.04 | 10.64 | 0.463 |
| TDCA | 7.40 | 5.76 | 0.2573 | T_β_MCA | 7.88 | 8.43 | 0.808 |
| β_MCA | 3.52 | 4.26 | 0.5430 | T_ω_MCA | 7.60 | 8.70 | 0.628 |
| TCDCA | 3.26 | 2.80 | 0.4568 | TCA | 6.48 | 7.26 | 0.714 |
| TUDCA | 3.04 | 2.68 | 0.3708 | 7oxoDCA | 5.00 | 3.15 | 0.352 |
| CA | 0.95 | 0.31 | 0.1452 | UDCA | 4.40 | 3.19 | 0.179 |
| ω_MCA | 0.87 | 0.97 | 0.8105 | T_α_MCA | 3.62 | 3.88 | 0.809 |
| TLCA | 0.83 | 1.28 | 0.1135 | CDCA | 2.27 | 1.83 | 0.435 |
| 7oxoDCA | 0.47 | 0.04 | 0.1751 | TDCA | 1.41 | 1.42 | 0.986 |
| α_MCA | 0.33 | 0.19 | 0.2326 | α_MCA | 1.30 | 0.92 | 0.148 |
| UDCA | 0.21 | 0.55 | 0.3787 | TUDCA | 1.28 | 0.81 | 0.093 |
| DCA | 0.11 | 0.04 | 0.1909 | HDCA | 0.73 | 0.75 | 0.911 |
| Tauro_7oxoLCA | 0.09 | 0.02 | 0.0601 | TCDCA | 0.57 | 0.53 | 0.857 |
| GCA | 0.04 | 0.04 | 0.8217 | GCA | 0.20 | 0.13 | 0.178 |
| CDCA | 0.03 | 0.02 | 0.4058 | TLCA | 0.16 | 0.16 | 0.976 |
| HDCA | 0.02 | 0.02 | 0.9130 | Tauro_7oxoLCA | 0.09 | 0.05 | 0.223 |

| | |
|---|---|
| 7 days BDL liver bile acid pool | 7 days BDL serum bile acid pool |

| | Cldn3^{+/+} | Cldn3^{-/-} | | | Cldn3^{+/+} | Cldn3^{-/-} | |
|---|---|---|---|---|---|---|---|
| | % of pool | % of pool | t test | | % of pool | % of pool | t test |
| T_β_MCA | 63.08 | 68.28 | 0.24 | TCA | 42.07 | 41.67 | 0.89 |
| TCA | 26.57 | 21.39 | 0.18 | T_β_MCA | 38.41 | 35.95 | 0.38 |
| T_ω_MCA | 7.02 | 7.02 | 1.00 | T_ω_MCA | 10.21 | 13.29 | 0.03 |
| T_α_MCA | 1.25 | 1.20 | 0.87 | TUDCA | 3.31 | 3.91 | 0.49 |
| TDCA | 0.64 | 0.61 | 0.82 | T_α_MCA | 3.02 | 2.83 | 0.82 |
| β_MCA | 0.50 | 0.62 | 0.41 | TCDCA | 1.48 | 1.43 | 0.84 |
| TUDCA | 0.43 | 0.39 | 0.58 | β_MCA | 0.83 | 0.57 | 0.26 |
| TCDCA | 0.42 | 0.42 | 0.97 | GCA | 0.21 | 0.12 | 0.03 |
| Tauro_7oxoLCA | 0.04 | 0.02 | 0.19 | α_MCA | 0.14 | 0.04 | 0.02 |
| GCA | 0.03 | 0.01 | 0.064 | CA | 0.12 | 0.07 | 0.39 |
| ω_MCA | 0.01 | 0.02 | 0.27 | Tauro_7oxoLCA | 0.12 | 0.14 | 0.63 |
| CA | 0.01 | 0.02 | 0.22 | ω_MCA | 0.10 | 0.18 | 0.11 |
| α_MCA | 0.01 | 0.01 | 0.53 | CDCA | 0.02 | 0.01 | 0.03 |
| | | | | TDCA | 0.02 | 0.03 | 0.56 |
| | | | | TLCA | 0.01 | 0.00 | 0.11 |
| | | | | DCA | 0.00 | 0.00 | 0.84 |

### Lower Expression of BA Synthesis- and Importer Genes in Cholestatic Cldn3^{-/-} Liver

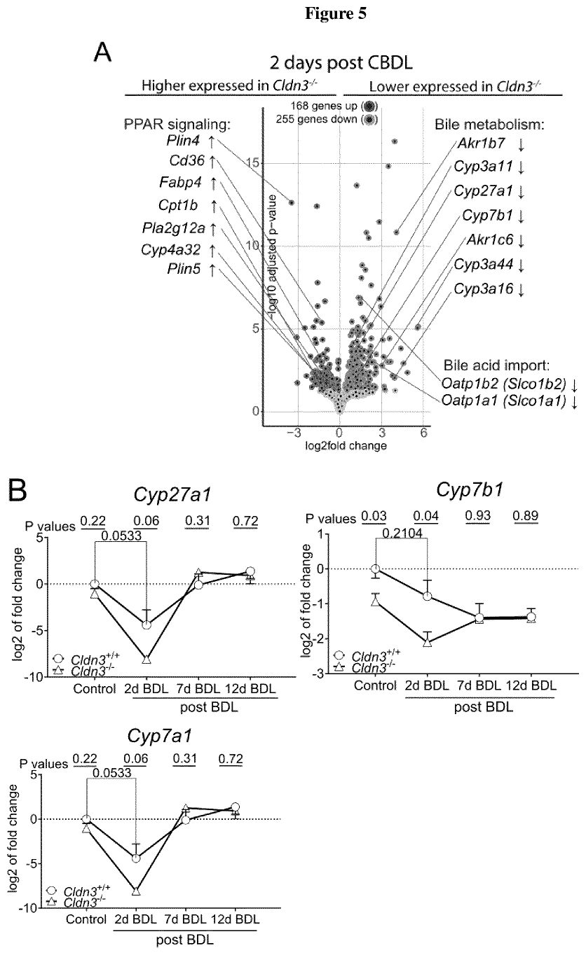

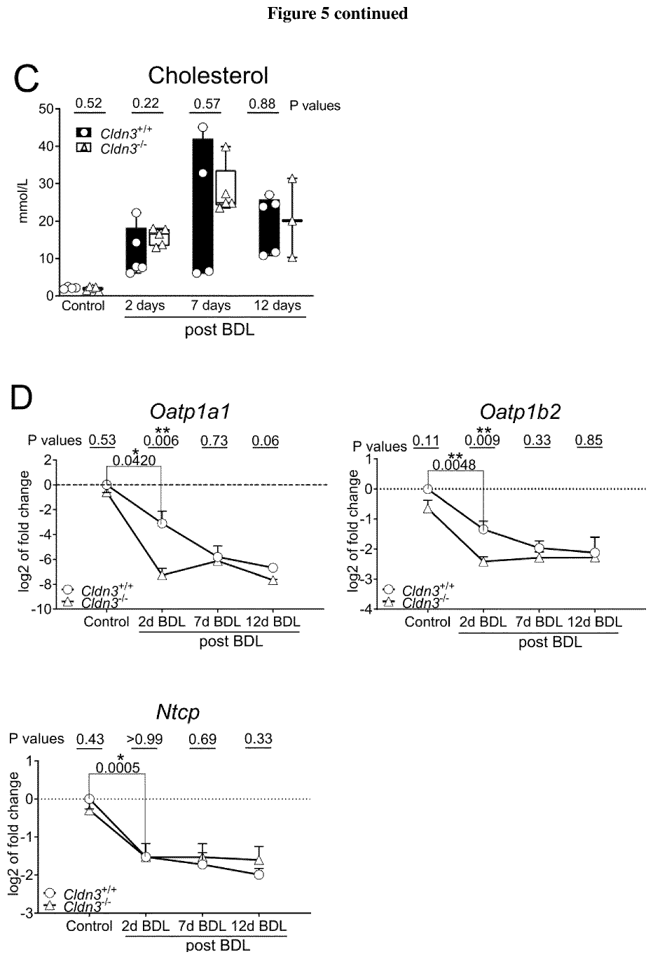

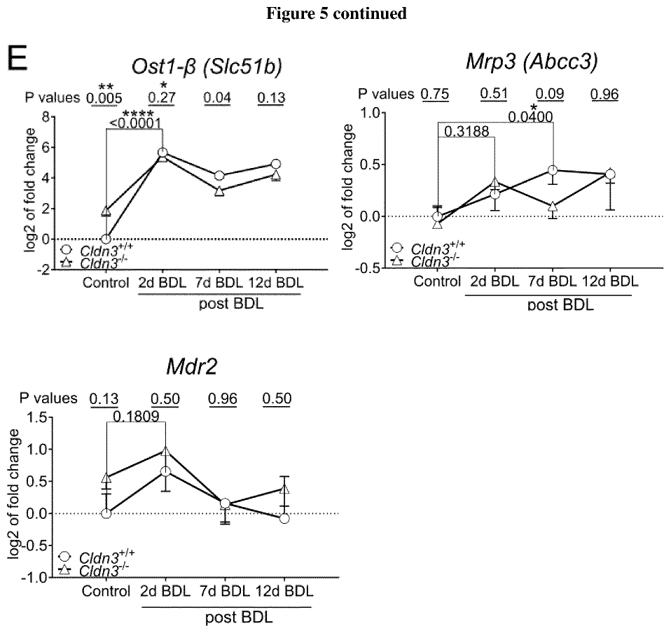

The observation that Cldn3^{-/-} bile is diluted and contains less BAs before- and after BDL raised the question if BAs synthesis and/or transport are changed. We sequenced the transcriptome of 2 days post BDL Cldn3^{-/-} and Cldn3^{+/+} liver tissue and performed a RT-qPCR on key genes to answer this question. Differential genes expression analysis showed that in Cldn3^{-/-} liver, there were 168 upregulated and 255 downregulated genes (P<0.05) (FIG. 5A).

We first performed a metascape analysis to obtain an overview on the gene pathways that differ most significantly when comparing Cldn3^{-/-} and Cldn3^{+/+} post BDL. (Table 3 and 4). Higher expressed in Cldn3^{-/-} liver were metabolism related pathways including amino acid catabolism fatty acid transport, carnitine metabolism. Interestingly, the cholestasis protective PPAR signalling pathway^{2,11,13} was also higher expressed in Cldn3^{-/-} liver (Table 3). Confirming the lower amount tissue injury and immune cell infiltration (FIG. 2B-H), we found several gene pathways related to inflammation and immune cell reaction to be significantly less expressed in Cldn3^{-/-} liver post BDL. This included leukocyte migration- and adhesion, reactive-oxygen-species biosynthesis, tumor necrosis factor biosynthesis and neutrophil degranulation that were lower expressed in absence of claudin-3 (Table 4). Importantly, the analysis showed that bile acid- and bile salt metabolism was among the top pathways that were less expressed in Cldn3^{-/-} mice (Table 4). The key lower expressed bile synthesis genes included Cyp7b1, Cyp27a1, Cyp3a11, Akr1b7, Akr1c6 and others (FIG. 5A). We confirmed this finding by qPCR for Cyp7b1 and Cyp27a1, which were significantly less expressed in control and at 2 days post BDL Cldn3^{-/-} liver (FIG. 5B). Cyp7a1 only showed a trend of lower expression at 2 days post BDL. Cholesterol as the precursor of bile acids did not differ in its blood levels in Cldn3^{-/-} mice (FIG. 5C).

TABLE 3
Upregulated gene pathways in Cldn3^{-/-}
liver, at two days post BDL

| Term | Pathway | LOG10(P) |
|---|---|---|
| GO: 0009068 | Aspartate family amino acid catabolic | −5.8236643 |
| | process | |
| GO: 0045646 | Regulation of erythrocyte | −4.465800406 |
| | differentiation | |
| mmu03320 | PPAR signaling pathway | −4.337144379 |
| GO: 0015908 | Fatty acid transport | −4.308436831 |
| GO: 0009437 | Carnitine metabolic process | −4.048398673 |
| GO: 0055080 | Cation homeostasis | −3.40397852 |
| GO: 0050873 | Brown fat cell differentiation | −3.321326758 |
| GO: 0051346 | Negative regulation of hydrolase | −3.069296423 |
| | activity | |
| GO: 0042136 | Neurotransmitter biosynthetic process | −2.965272306 |
| GO: 0045926 | Negative regulation of growth | −2.905887479 |
| GO: 0060393 | Regulation of pathway-restricted SMAD | −2.899551204 |
| | protein phosphorylation | |
| GO: 0010942 | Positive regulation of cell death | −2.731542462 |
| GO: 0043277 | Apoptotic cell clearance | −2.595002343 |
| GO: 0008277 | Regulation of G protein-coupled | −2.560984921 |
| | receptor signaling pathway | |
| R-MMU- | O-glycosylation of TSR domain- | −2.560680595 |
| 5173214 | containing proteins | |

TABLE 4
Downregulated gene pathways in Cldn3^{-/-} liver,
at two days post BDL

| Term | Pathway | LOG10(P) |
|---|---|---|
| mmu05204 | Chemical carcinogenesis | −22.63113735 |
| GO: 0032787 | monocarboxylic acid metabolic process | −11.83431298 |
| mmu00982 | Drug metabolism - cytochrome P450 | −10.26435367 |
| mmu00380 | Tryptophan metabolism | −6.920345124 |
| GO: 0008610 | lipid biosynthetic process | −6.831374008 |
| GO: 0050900 | leukocyte migration | −5.882886828 |
| GO: 1903409 | reactive oxygen species biosynthetic | −5.706864744 |
| | process | |
| GO: 0007159 | leukocyte cell-cell adhesion | −5.511383797 |
| mmu00983 | Drug metabolism - other enzymes | −5.399776811 |
| R-MMU- | Bile acid and bile salt metabolism | −5.399776811 |
| 194068 | | |
| mmu04145 | Phagosome | −5.284454092 |
| R-MMU- | RA biosynthesis pathway | −5.280601012 |
| 5365859 | | |
| GO: 0042533 | tumor necrosis factor biosynthetic | −5.222788774 |
| | process | |
| GO: 0035634 | response to stilbenoid | −5.115412514 |
| R-MMU- | Hemostasis | −4.974591528 |
| 109582 | | |

We also checked the expression of BA transporters. As expected, the expression of basolateral BA importers Oatp1a1, Oatp1b2 and Ntcp was downregulated in response to the BDL in wildtype animals, however Oatp1a1/Oatp1b2 decreased significantly more in the Cldn3^{-/-} livers at 2 days post BDL (FIG. 5D). The basolateral BA exporter Ost1-β was higher expressed in control Cldn3^{-/-} liver which confirmed our previous findings²⁷. Ost1-β expression was strongly upregulated in response to the BDL, which happened similarly in Cldn3^{+/+} and Cldn3^{-/-} livers (FIG. 5E). The apical anion and bile acid exporter Mpr3 was slightly upregulated at 7 days post BDL in Cldn3^{+/+} but not in Cldn3^{-/-} livers (FIG. 5E). Lastly, the apical biliary phospholipid exporter Mdr2 was overall not significantly regulated following BDL (FIG. 5E).

Overall, the gene expression analysis confirmed the lower inflammation and showed that BA synthesis genes are less expressed. In agreement with our observation of impaired bile acid-sized tracer uptake (FIG. 1H-L), we found an impaired expression of bile acid importers in Cldn3^{-/-} livers.

Cldn3^{-/-} mice have Increased Periductal Oedema but not Change in Ductular Reaction

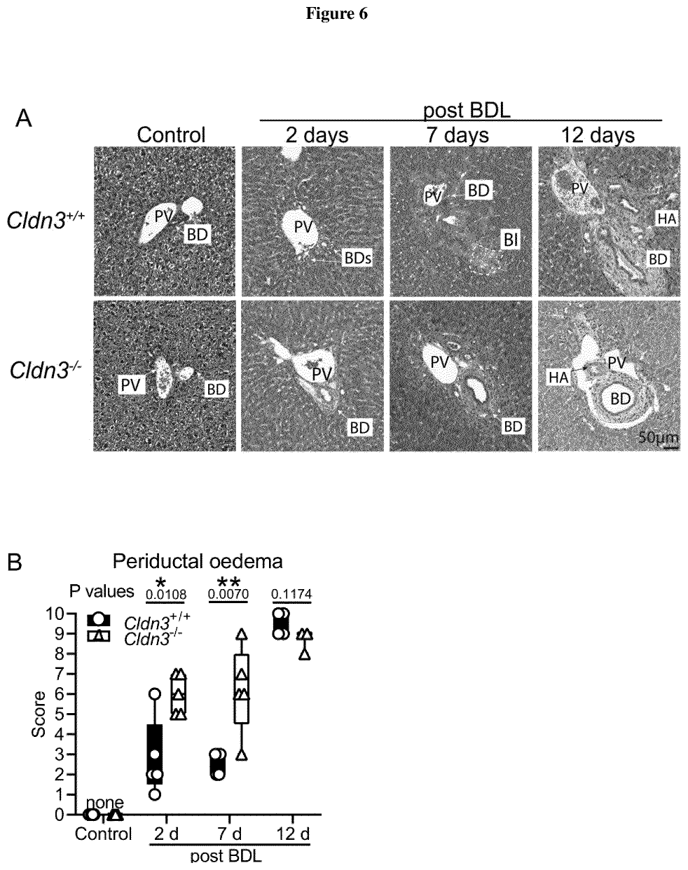

To understand the amelioration of cholestatic injury in Cldn3^{-/-} mice we described the tissue morphology of wildtype and claudin-3 deficient mice post BDL in more detail. Since claudin-3 is highly expressed in cholangiocytes²⁷ and loss of claudin-3 has been previously associated with an impaired of the paracellular barrier for water^{30,31}, we scored hematoxylin and eosin stained liver sections for oedema in periductal zones. Average periductal oedema scores in Cldn3^{+/+} animals were 2.8 compared to 6 in Cldn3^{-/-} (P<0.05) at 2 days post BDL (FIG. 6A, B). We also observed higher ductal oedema in knockout mice at the seven-day time point. Oedema scores were highest after 12 days BDL but did not differ significantly among groups.

Taken together, the histological analysis showed that Cldn3^{-/-} livers have increased periductal oedema following BDL as the possible result of a leaky water barrier.

Cldn12′-Animals are not Protected from Cholestatic Liver Injury

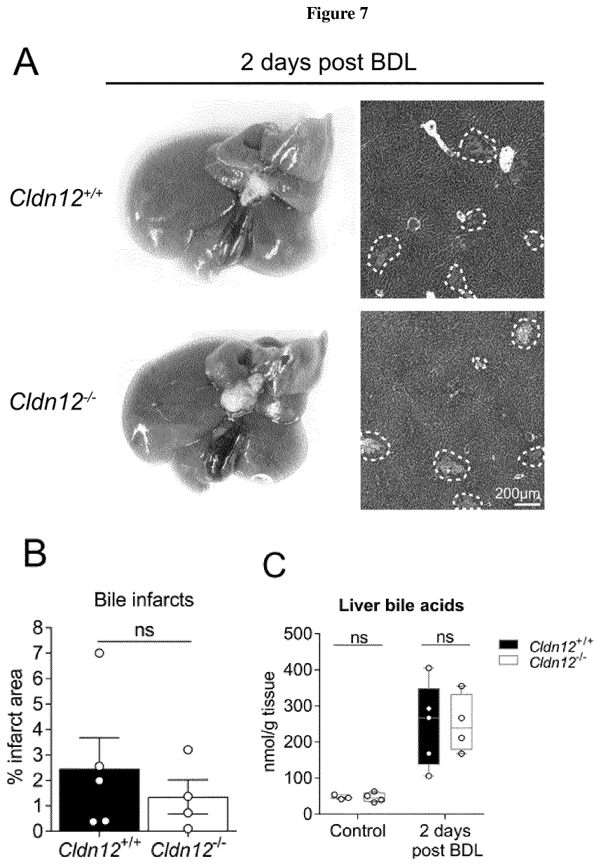

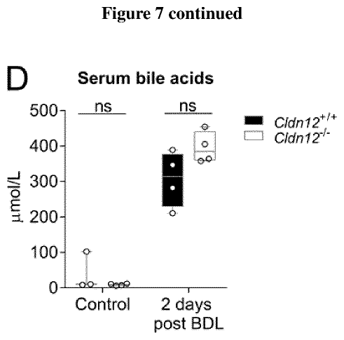

We next asked the question whether cholestasis protection could also be achieved by knocking out another claudin family tight junction protein. We recently described the cell type specific expression of hepatic tight junction genes and found that Cldn12 is expressed in murine hepatocytes²⁷. Therefore, we subjected C57BL/6 mice with global claudin-12 loss to BDL. Cldn12^{+/+} and Cldn12^{-/-} livers similarly developed enlarged and -dark coloured gallbladders 2 days post BDL (FIG. 7A). Quantification of the tissue necrosis area showed that both groups had similar levels of tissue necrosis (FIG. 7A, B). Finally, the concentrations of liver- or serum TBAs were not altered in Cldn12^{-/-} mice (FIGS. 7C and D). These results show that loss of claudin-12 does not lead to changed bile acid levels and does not ameliorate of cholestatic liver injury.

Claudin-3 Loss Protects from Intrahepatic Cholestasis

The promising results from the BDL model raised the question if loss of claudin-3 could also be of benefit in intrahepatic cholestasis diseases. We therefor challenged Cldn3^{-/-} mice with the well-established model of oral α-Naphthylisothiocyanate (ANIT) administration³².

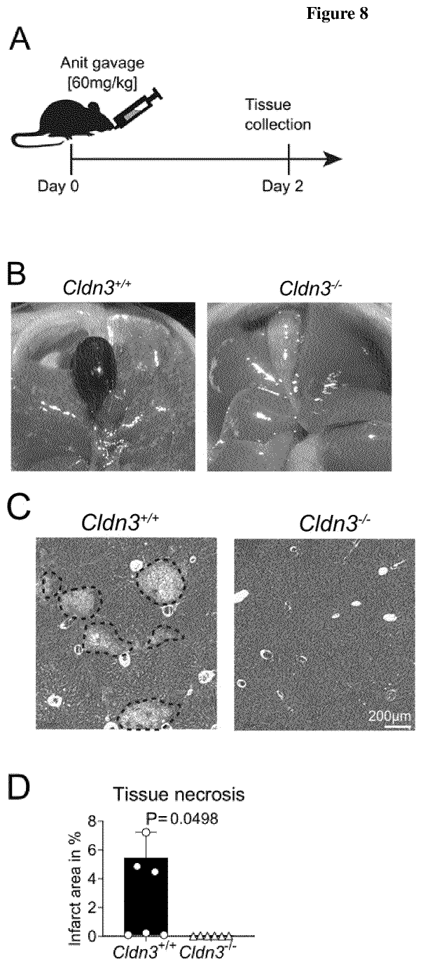

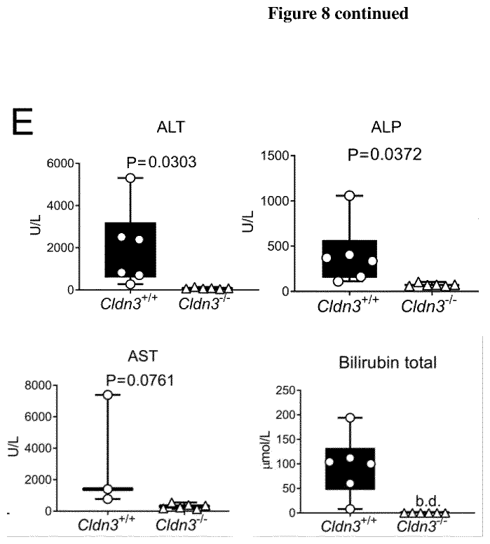

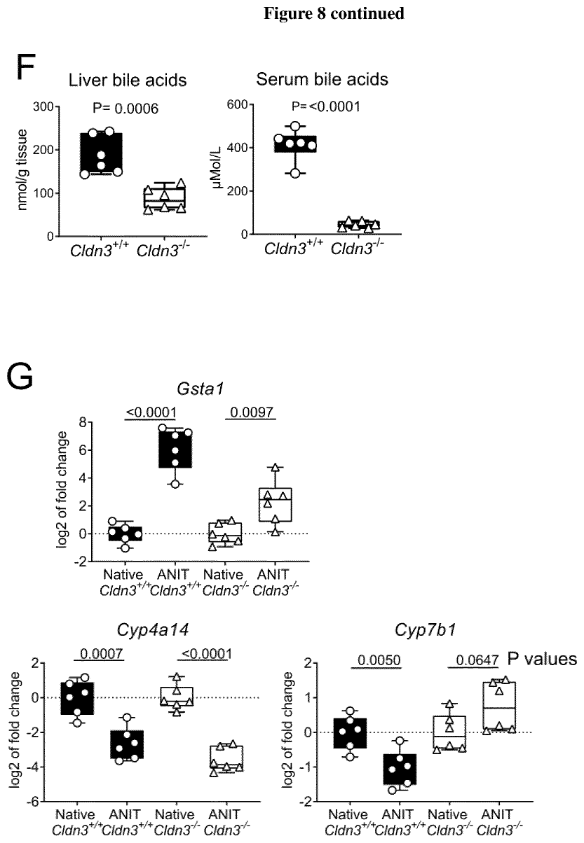

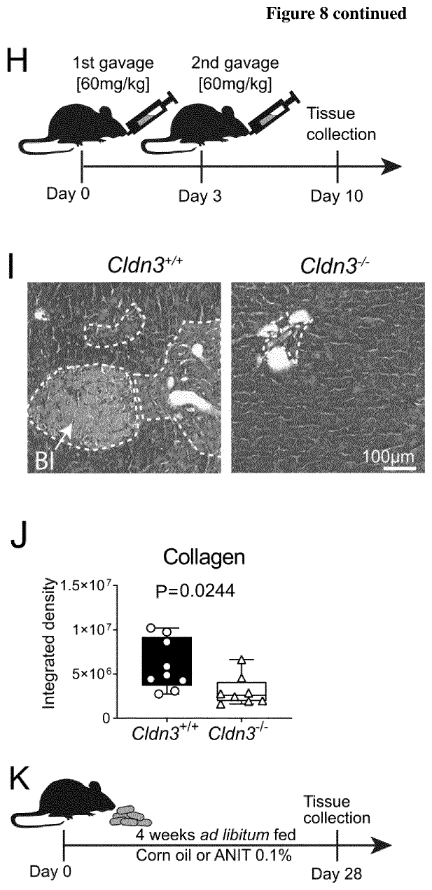

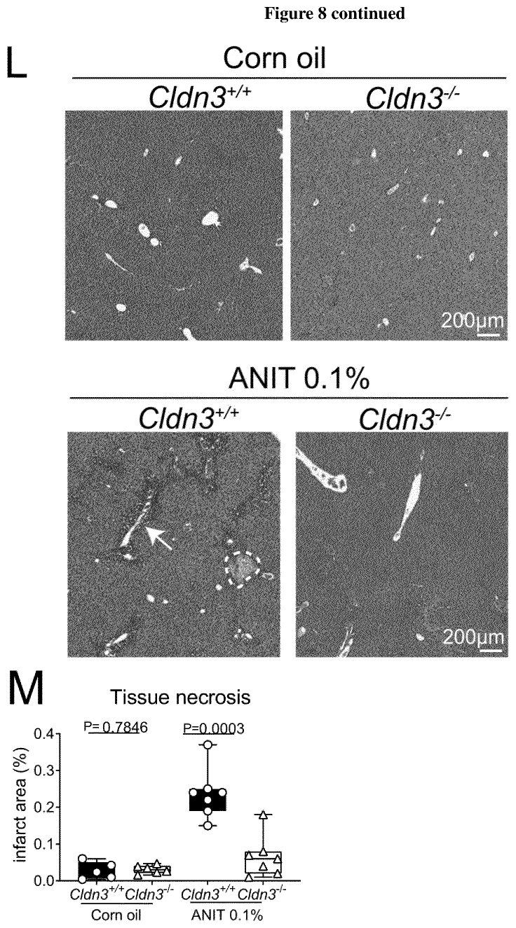

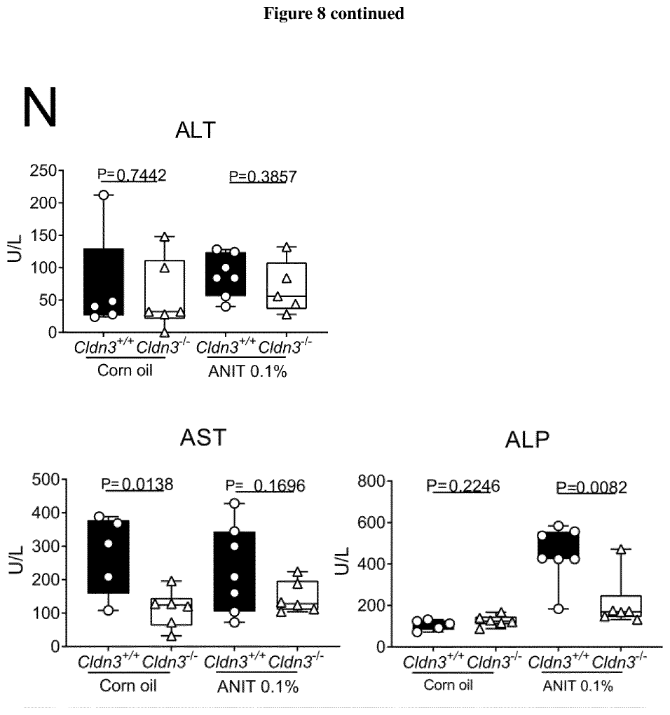

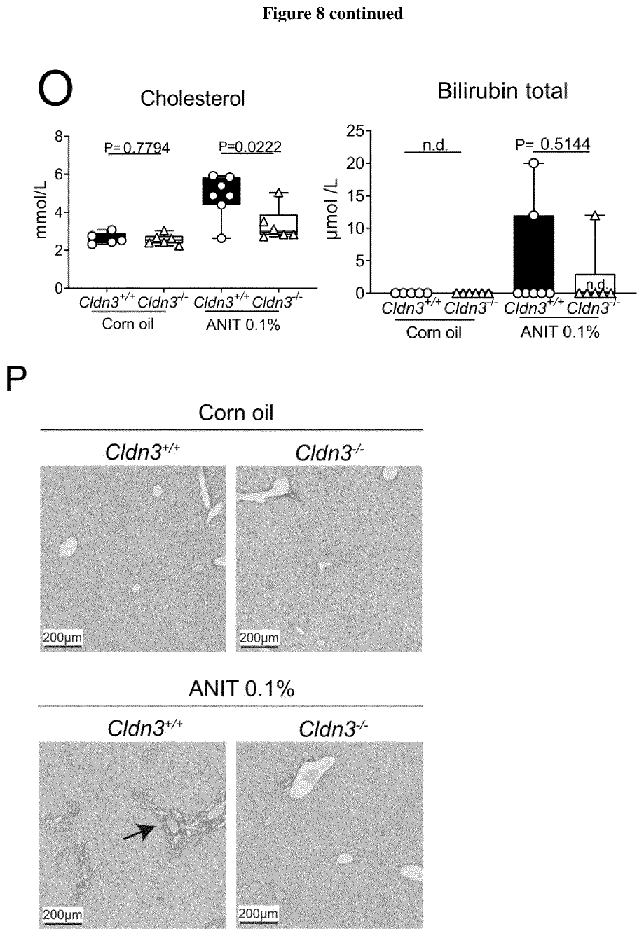

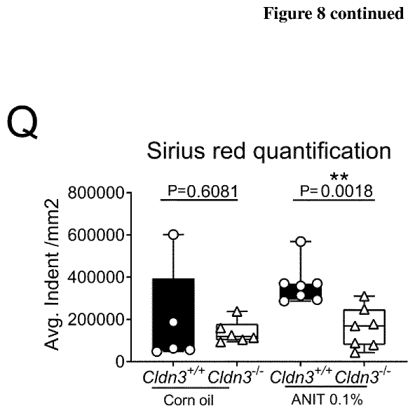

First, we tested a model of acute intrahepatic cholestasis. We challenged the mice with a single dose of ANIT [60 mg/KG bodyweight] and analysed the tissues after two days (FIG. 8A). In wildtype mice, the expected phenotype pf cholestatic liver injury was induced, with macro- and microscopic visible tissue necrosis (FIG. 8B-D), high levels of clinical liver injury markers (FIG. 8E) as well as elevated bilirubin and bile acid levels in tissue and serum (FIGS. 8E and F). Strikingly, there was a complete absence of tissue necrosis in Cldn3^{-/-} livers (FIG. 8B-D). The bile contained in the gallbladder had a normal light-yellow colour (appears lighter/white in the grayscale figure) in Cldn3^{-/-} mice, in contrast to the dark green bile in ANIT challenged Cldn3^{+/+} (FIG. 8B, appears dark/black in the grayscale figure). Blood ALT ALP and bilirubin levels were significantly lower in Cldn3^{-/-} animals (P<0.05) and there was a trend for lower AST levels as well (FIG. 8E). Evidentiary for the absence of cholestatic injury were also the significantly lower BA levels. Liver total BAs were on average 188 nmol/g in Cldn3^{+/+} and only 87 nmol/g in Cldn3^{-/-} livers (P<0.001) (FIG. 8F). Striking was also the difference in serum BA levels, with a mean of 412 μmol/L in Cldn3^{+/+} and only 46 μmol/L in Cldn3^{-/-} mice (P<0.0001) (FIG. 8F).

We questioned whether ANIT is metabolised similarly by Cldn3^{+/+} and Cldn3^{-/-} livers and therefor checked the expression of glutathione metabolism and drug processing involved genes glutathione S-transferase alpha 1 (Gsta1) and Cyp4a12. Gsta1 as expected was upregulated 2 days post ANIT, however less so in Cldn3^{-/-} livers (FIG. 8G). Cyp4a14 was downregulated similarly in both groups in response to ANIT. In representation of a negative feedback to cholestasis, the key BA synthesis enzyme Cyp7b1 was downregulated as expected in Cldn3^{+/+} mice. However this was not the case in absence in claudin-3, indicating less bile acid induced feedback and showing the protection from of cholestasis (FIG. 8G). To check if loss of claudin-3 also protects from the fibrosis that prolonged intrahepatic cholestasis can induce, we subjected mice to two subsequent doses of ANIT for a total duration of 10 days (FIG. 8H). Quantification of the collagen intensity of masson-trichrome stained liver sections showed that Cldn3^{-/-} livers contain significantly less collagen than wildtype mice (FIGS. 8I and J).

Lastly, we tested the protective effect of claudin-3 loss during chronic intrahepatic cholestasis using a model of 4 week ad libitum feeding of 0.1% ANIT (FIG. 8K). Mice of the control group were fed with chow containing same volumes of corn oil instead. As expected, the control group mice never raised any tissue necrosis, clinical injury markers and bilirubin/cholesterol levels remained normal (FIG. 8L-O). At 4 weeks post ANIT feeding, Cldn3^{+/+} mice developed clear signs of ductular reaction and tissue necrosis, which was not the case in Cldn3^{-/-} tissue (FIG. 8L). The quantification of tissue necrosis showed that Cldn3^{+/+} livers had a mean of 0.24% total necrosis area, whereas Cldn3^{-/-} livers only had 0.066% (P<0.001) (FIG. 8M). ALP levels as clinical markers of cholestatic liver injury were 448 U/L in ANIT fed Cldn3^{+/+} mice but only only 211 U/L in the corresponding Cldn3^{-/-} group (P<0.01) (FIG. 8N). AST and ALT levels did not differ among the ANIT fed groups (FIG. 8N). While bilirubin levels did not differ, cholesterol levels were significantly lower in Cldn3^{-/-} serum (P<0.05) (FIG. 8O).

We next quantified the amount of fibrotic liver tissue by staining with Sirius red. Mice that were fed with corn oil showed no fibrosis as indicated by the minimal Sirius red stained tissue (FIG. 8P). Cldn3^{+/+} mice fed for 4 weeks with 0.1% ANIT developed fibrosis as shown by the profound amount of collagen deposition in the Sirius red stained sections, which was not the case for Cldn3^{-/-} mice (FIG. 8P). Quantification of the Sirius red signal (FIG. 8Q) confirmed that ANIT fed Cldn3^{-/-} mice did not develop any liver fibrosis as shown by the significantly lower amount of Sirius red signal, when compared to ANIT fed Cldn3^{+/+} mice (P=0.0018, students t test).

Collectively, the experiments using the acute- and chronic intrahepatic cholestasis models show that Cldn3^{-/-} are protected from cholestatic liver injury and fibrosis associated with cholestasis.

GalNAc siRNA Targeting of Claudin-3 Achieves Modest Amelioration of Cholestatic Injury

The promising results that we obtained with the Cldn3^{-/-} mice suggest that claudin-3 targeting could be beneficial for cholestasis therapy. To proof this concept, we knocked down (KD) claudin-3 using GalNAc siRNAs. The advantages of this technique are that it produces a stable KD of the target, can be administered subcutaneously, only targets liver hepatocytes and is already in used in clinics³³. We first screened 12-candidate siRNAs in-vitro using primary mouse hepatocytes. A siRNA (SEQ ID NO: 3, sense strand) targeting activator of heat shock protein ATPase 1 (AHSA1) was used as a negative control in the subsequent experiments. The western blot showed that candidate 9- and 12 produce a strong KD of claudin-3 when we treated the cells at a concentration of 50 nmol. We therefor continued to use candidate 9 (from here on termed “siRNA1”; SEQ ID NO:1, sense strand) and candidate 12 (from here on termed “siRNA2”; SEQ ID NO: 2, sense strand) in a model of obstructive cholestasis, using the BDL technique.

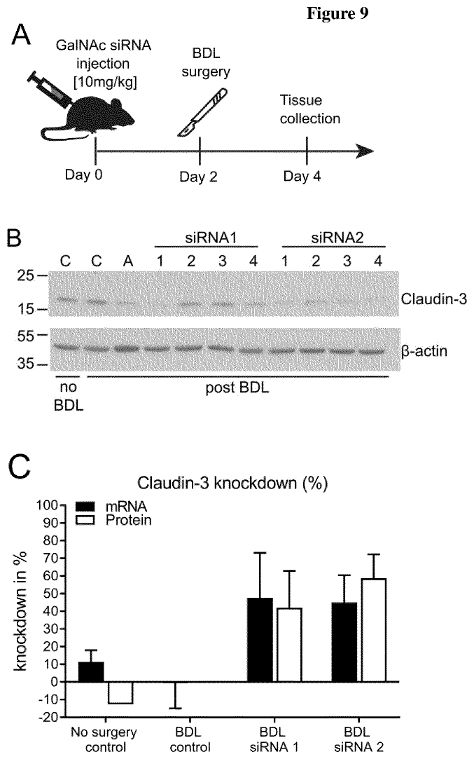

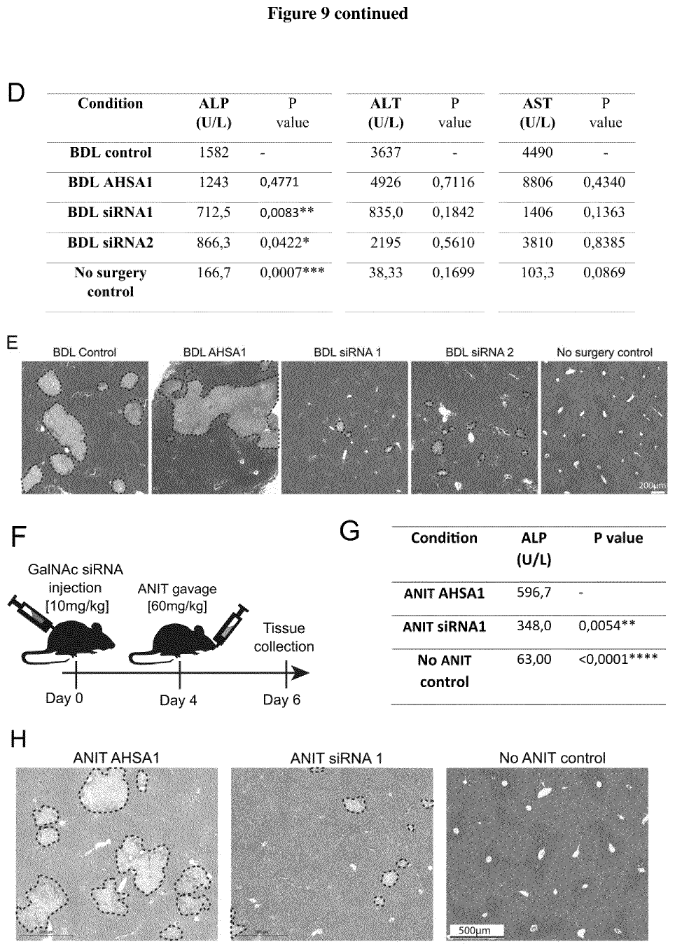

C57BL/6 wildtype mice were subcutaneously injected with GalNAc siRNA1- or siRNA2 at 10 mg/KG bodyweight. Two days post injection, BDL was performed. Then two days post-surgery, tissues were collected and analysed for claudin-3 levels and markers of cholestatic liver injury (FIG. 9A). The in-vivo claudin-3 KD efficacy of the siRNAs was about 40-50% on mRNA and proteins levels, however the individual differences in efficacy were substantial (FIGS. 9B and C). Nevertheless, the claudin-3 KD resulted in significantly reduced ALP (FIG. 9D and Table 5). ALP levels of control mice with BDL had a mean of 1582 U/L, whereas the mean of siRNA1 or siRNA2 receiving animals was at 713 and 866 U/L, respectively (P<0.01 and P<0.05, respectively) (FIG. 9D and Table 5). Similarity ALT and AST levels were reduced compared to control groups (FIG. 9D and Table 5). Representative H&E-stained liver tissue sections confirmed the amelioration in tissue injury in the claudin-3 knockdown groups, however the effects were less striking and the variability among individuals remained high (FIG. 9E).

TABLE 5
Serum markers of cholestasis- and liver injury after 2 days BDL. P values were calculated
comparing the corresponding condition to the BDL control group in the first row (n =
3 for BDL-control and no-surgery control, n = 4 for BDL plus siRNA groups, student's t test)

| Condition | ALP (U/L) | P value | ALT (U/L) | P value | AST (U/L) | P value |
|---|---|---|---|---|---|---|
| BDL control | 1582 | — | 3637 | — | 4490 | — |
| BDL AHSA1 | 1243 | 0.4771 | 4926 | 0.7116 | 8806 | 0.4340 |
| BDL siRNA1 | 712.5 | 0.0083** | 835.0 | 0.1842 | 1406 | 0.1363 |
| BDL siRNA2 | 866.3 | 0.0422* | 2195 | 0.5610 | 3810 | 0.8385 |
| No surgery control | 166.7 | 0.0007*** | 38.33 | 0.1699 | 103.3 | 0.0869 |

Next, we tested our most promising siRNA candidate, siRNA1, in the intrahepatic cholestasis model using ANIT. We injected either control siRNA AHSA1 or claudin-3 targeting siRNA1 subcutaneously at four days prior to the oral gavage of ANIT [60 mg/KG bodyweight] (FIG. 9F). Two days after ANIT gavage, tissues and serum were collected to quantify the liver damage. We found that animals treated with anti claudin-3 siRNA1 had significantly lower levels of the main clinical blood marker for cholestasis, that is ALP (FIG. 9G and Table 6). Accordingly, the liver tissue histology showed less necrosis in the animals that received anti claudin-3 siRNA1, when compared to the AHSA1 control siRNA (FIG. 9H).

TABLE 6
Serum markers of cholestasis- and liver injury after 2
days ANIT. P values were calculated comparing the corresponding
condition to the ANIT AHSA1 control siRNA group in the
first row (n = 3 for ASHA-control, n = 5 for
ANIT siRNA1, n = 4 for No ANIT control, student's t test)

| | ALP | |
|---|---|---|
| Condition | (U/L) | P value |
| ANIT AHSA1 | 596.7 | — |
| ANIT siRNA1 | 348.0 | 0.0054** |
| No ANIT | 63.00 | <0.0001**** |
| control | | |

Taken together, we show that targeted GalNAc siRNA knockdown of claudin-3 leads to an amelioration of extra- or intrahepatic cholestatic liver injury.

Screening, Synthesis and Evaluation of siRNAs Targeting Human Claudin-3

As as first step to translate these findings to human patients, we next screened for siRNA sequences that efficiently knockdown human claudin-3. In the bionformatic pre-selection (Axolabs, Kulmbach, Germany), siRNAs were scored for target specificity, intra- and inter-species cross-reactivity and activity. The best siRNA candidates were then synthetized (Axolabs, Kulmbach, Germany). siRNA sequences contained chemically stabilising additions of 2′-fluoro- or 2′O-methyl modifications, or phosphorothioate linkers at the positions indicated in table 6.

Next, the synthetized siRNA candidates were evaluated for their efficiency to knockdown human claudin-3, in cultures of the human liver cell line PLC (ATCC Cat. No CRL-8024). The human cell cultures were treated with a final concentration of 50 nm siRNA, and the RNA was extracted after two days of treatment. Finally, RT-qPCR was used to quantify the amount of remaining human claudin-3 mRNA after siRNA treatment. Table 6 lists all human siRNA sequences that knocked down equal- or more than 50% of human claudin-3 mRNA. Table 7 displays the same identified human claudin-3 siRNA sequences, without their chemically stabilizing modifications.

TABLE 6
Sequences of siRNAs that knockdown 50% or more of human claudin-3 mRNA,
identified within the in vitro cell culture experiment. The experiment
was done twice, resulting in comparable results.

| | | | Seq | |
|---|---|---|---|---|
| SIRNA | Seq | | ID | |
| ID | ID No. | sense strand sequence (5′-3′) | No | antisense strand sequence (5′-3′) |
| 1 | 33 | csasacauCfaUfCfAfcgucgcagaa | 67 | usUfscugCfgacgugaUfgAfuguugscsu |
| 2 | 34 | ascscaacUfgCfGfUfgcaggacgaa | 68 | usUfscguCfcugcacgCfaGfuuggusgsc |
| 3 | 35 | gsgsccaaCfaCfCfAfuuauccggga | 69 | usCfsccgGfauaauggUfgUfuggccsgsa |
| 4 | 36 | csgscagaAfgCfGfCfgagaugggca | 70 | usGfscccAfucucgcgCfuUfcugcgscsc |
| 5 | 37 | csasgaagCfgCfGfAfgaugggcgca | 71 | usGfscgcCfcaucucgCfgCfuucugscsg |
| 6 | 38 | csgscgagAfaGfAfAfguacacggca | 72 | usGfsccgUfguacuucUfuCfucgcgsusg |
| 7 | 39 | ascscaagGfuCfGfUfcuacuccgca | 73 | usGfscggAfguagacgAfcCfuuggusgsg |
| 8 | 40 | asasggucGfuCfUfAfcuccgcgcca | 74 | usGfsgcgCfggaguagAfcGfaccuusgsg |
| 9 | 41 | asgsgucgUfcUfAfCfuccgcgccga | 75 | usCfsggcGfcggaguaGfaCfgaccususg |
| 10 | 42 | gsgsucguCfuAfCfUfccgcgccgca | 76 | usGfscggCfgcggaguAfgAfcgaccsusu |
| 11 | 43 | csusgggcAfcAfGfGfcuacgaccga | 77 | usCfsgguCfguagccuGfuGfcccagsgsc |
| 12 | 44 | usgsggcaCfaGfGfCfuacgaccgca | 78 | usGfscggUfcguagccUfgUfgcccasgsg |
| 13 | 45 | gsgscacaGfgCfUfAfcgaccgcaaa | 79 | usUfsugcGfgucguagCfcUfgugccscsa |
| 14 | 46 | gscsacagGfcUfAfCfgaccgcaaga | 80 | usCfsuugCfggucguaGfcCfugugcscsc |
| 15 | 47 | csusacgaCfcGfCfAfaggacuacga | 81 | usCfsguaGfuccuugcGfgUfcguagscsc |
| 16 | 48 | ascsgaccGfcAfAfGfgacuacguca | 82 | usGfsacgUfaguccuuGfcGfgucgusasg |
| 17 | 49 | csgsaccgCfaAfGfGfacuacgucua | 83 | usAfsgacGfuaguccuUfgCfggucgsusa |
| 18 | 50 | cscsgcaaGfgAfCfUfacgucuaaga | 84 | usCfsuuaGfacguaguCfcUfugcggsusc |
| 19 | 51 | csusacguCfuAfAfGfggacagacga | 85 | usCfsgucUfgucccuuAfgAfcguagsusc |
| 20 | 52 | ascsgucuAfaGfGfGfacagacgcaa | 86 | usUfsgcgUfcugucccUfuAfgacgusasg |
| 21 | 53 | csgsucuaAfgGfGfAfcagacgcaga | 87 | usCfsugcGfucuguccCfuUfagacgsusa |
| 22 | 54 | csusggagCfgCfGfCfaccaggccaa | 88 | usUfsggcCfuggugcgCfgCfuccagscsu |
| 23 | 55 | ususgcggGfcCfGfGfgcagucgaca | 89 | usGfsucgAfcugcccgGfcCfcgcaasasg |
| 24 | 56 | gscscgggCfaGfUfCfgacuucggga | 90 | usCfsccgAfagucgacUfgCfccggcscsc |
| 25 | 57 | cscsagggAfcCfAfAfccugcaugga | 91 | usCfscauGfcagguugGfuCfccuggsgsc |
| 26 | 58 | asusggacUfgUfGfAfaaccucacca | 92 | usGfsgugAfgguuucaCfaGfuccausgsc |
| 27 | 59 | gsusgaccGfcCfAfAfuacuugacca | 93 | usGfsgucAfaguauugGfcGfgucacscsc |
| 28 | 60 | gsasccgcCfaAfUfAfcuugaccaca | 94 | usGfsuggUfcaaguauUfgGfcggucsasc |
| 29 | 61 | cscsaauaCfuUfGfAfccaccccgua | 95 | usAfscggGfguggucaAfgUfauuggscsg |
| 30 | 62 | asasuacuUfgAfCfCfaccccgucga | 96 | usCfsgacGfgggugguCfaAfguauusgsg |
| 31 | 63 | asusacuuGfaCfCfAfccccgucgaa | 97 | usUfscgaCfgggguggUfcAfaguaususg |
| 32 | 64 | ascsuugaCfcAfCfCfccgucgagca | 98 | usGfscucGfacgggguGfgUfcaagusasu |
| 33 | 65 | ascscccgUfcGfAfGfccccaucgga | 99 | usCfscgaUfggggcucGfaCfggggusgsg |
| 34 | 66 | cscscgucGfaGfCfCfccaucgggca | 100 | usGfscccGfauggggcUfcGfacgggsgsu |

Legend:
n = 2′O-methyl RNA
Nf = 2′-fluoro RNA
s = phosphorothioate

TABLE 7
Sequences of siRNAs that knockdown 50% or more of human claudin-3 mRNA,
displayed without modifications.

| | Seq | | | |
|---|---|---|---|---|
| siRNA | ID | sense strand sequence | Seq ID | antisense strand |
| ID | No. | (5′-3′) | No. | sequence (5′-3′) |
| 1 | 101 | CAACAUCAUCACGUCGCAGAA | 135 | UUCUGCGACGUGAUGAUGUUGCU |
| 2 | 102 | ACCAACUGCGUGCAGGACGAA | 136 | UUCGUCCUGCACGCAGUUGGUGC |
| 3 | 103 | GGCCAACACCAUUAUCCGGGA | 137 | UCCCGGAUAAUGGUGUUGGCCGA |
| 4 | 104 | CGCAGAAGCGCGAGAUGGGCA | 138 | UGCCCAUCUCGCGCUUCUGCGCC |
| 5 | 105 | CAGAAGCGCGAGAUGGGCGCA | 139 | UGCGCCCAUCUCGCGCUUCUGCG |
| 6 | 106 | CGCGAGAAGAAGUACACGGCA | 140 | UGCCGUGUACUUCUUCUCGCGUG |
| 7 | 107 | ACCAAGGUCGUCUACUCCGCA | 141 | UGCGGAGUAGACGACCUUGGUGG |
| 8 | 108 | AAGGUCGUCUACUCCGCGCCA | 142 | UGGCGCGGAGUAGACGACCUUGG |
| 9 | 109 | AGGUCGUCUACUCCGCGCCGA | 143 | UCGGCGCGGAGUAGACGACCUUG |
| 10 | 110 | GGUCGUCUACUCCGCGCCGCA | 144 | UGCGGCGCGGAGUAGACGACCUU |
| 11 | 111 | CUGGGCACAGGCUACGACCGA | 145 | UCGGUCGUAGCCUGUGCCCAGGC |
| 12 | 112 | UGGGCACAGGCUACGACCGCA | 146 | UGCGGUCGUAGCCUGUGCCCAGG |
| 13 | 113 | GGCACAGGCUACGACCGCAAA | 147 | UUUGCGGUCGUAGCCUGUGCCCA |
| 14 | 114 | GCACAGGCUACGACCGCAAGA | 148 | UCUUGCGGUCGUAGCCUGUGCCC |
| 15 | 115 | CUACGACCGCAAGGACUACGA | 149 | UCGUAGUCCUUGCGGUCGUAGCC |
| 16 | 116 | ACGACCGCAAGGACUACGUCA | 150 | UGACGUAGUCCUUGCGGUCGUAG |
| 17 | 117 | CGACCGCAAGGACUACGUCUA | 151 | UAGACGUAGUCCUUGCGGUCGUA |
| 18 | 118 | CCGCAAGGACUACGUCUAAGA | 152 | UCUUAGACGUAGUCCUUGCGGUC |
| 19 | 119 | CUACGUCUAAGGGACAGACGA | 153 | UCGUCUGUCCCUUAGACGUAGUC |
| 20 | 120 | ACGUCUAAGGGACAGACGCAA | 154 | UUGCGUCUGUCCCUUAGACGUAG |
| 21 | 121 | CGUCUAAGGGACAGACGCAGA | 155 | UCUGCGUCUGUCCCUUAGACGUA |
| 22 | 122 | CUGGAGCGCGCACCAGGCCAA | 156 | UUGGCCUGGUGCGCGCUCCAGCU |
| 23 | 123 | UUGCGGGCCGGGCAGUCGACA | 157 | UGUCGACUGCCCGGCCCGCAAAG |
| 24 | 124 | GCCGGGCAGUCGACUUCGGGA | 158 | UCCCGAAGUCGACUGCCCGGCCC |
| 25 | 125 | CCAGGGACCAACCUGCAUGGA | 159 | UCCAUGCAGGUUGGUCCCUGGGC |
| 26 | 126 | AUGGACUGUGAAACCUCACCA | 160 | UGGUGAGGUUUCACAGUCCAUGC |
| 27 | 127 | GUGACCGCCAAUACUUGACCA | 161 | UGGUCAAGUAUUGGCGGUCACCC |
| 28 | 128 | GACCGCCAAUACUUGACCACA | 162 | UGUGGUCAAGUAUUGGCGGUCAC |
| 29 | 129 | CCAAUACUUGACCACCCCGUA | 163 | UACGGGGUGGUCAAGUAUUGGCG |
| 30 | 130 | AAUACUUGACCACCCCGUCGA | 164 | UCGACGGGGUGGUCAAGUAUUGG |
| 31 | 131 | AUACUUGACCACCCCGUCGAA | 165 | UUCGACGGGGUGGUCAAGUAUUG |
| 32 | 132 | ACUUGACCACCCCGUCGAGCA | 166 | UGCUCGACGGGGUGGUCAAGUAU |
| 33 | 133 | ACCCCGUCGAGCCCCAUCGGA | 167 | UCCGAUGGGGCUCGACGGGGUGG |
| 34 | 134 | CCCGUCGAGCCCCAUCGGGCA | 168 | UGCCCGAUGGGGCUCGACGGGGU |

The experiment was done twice, resulting in comparable results. In summary, we identified 34 siRNA sequences that knocked down human claudin-3 with an efficacy of equal- or greater than 50%. The best performing siRNAs will be now used further for confirming- and de-risking studies, following a typical drug development process.

### Depletion of Human Claudin-3 Protein Using a Monoclonal Antibody

To prove that human claudin-3 inhibition can be achieved with another pharmaceutic modality, we generated- and functionalized a human claudin-3 antibody (light chain as shown in SEQ ID NO: 169 and heavy chain as shown in SEQ ID NO: 170) bound to GalNAc. Our claudin-3 antibody bound efficiently to human claudin-3 in a liver cell line and inhibited the target in a dose-dependent manner, which is described in more detail in the following.

### Binding of Anti Claudin-3 Antibody UB-VS003 Against Human and Mouse Cells

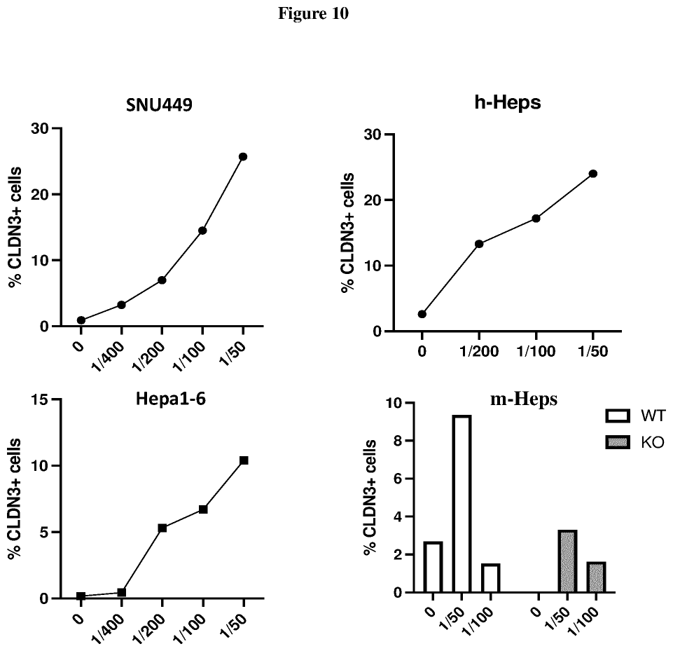

FIG. 10 presents the binding analysis of the anti claudin-3 antibody UB-VS003 against Human SNU449 cells and hepatocytes, as well as mouse Hepa1-6 cells. The results demonstrate a consistent trend in antibody binding for all cell types.

For Human SNU449 cells and hepatocytes, the control samples showed no detectable binding (0%). However, as the antibody concentration increased, a progressive increase in binding was observed. At a dilution of 1/400, the binding percentage was approximately 3%. This increased to 7% at a dilution of 1/200, 12% at 1/100, and reached a maximum of 25% at 1/50 dilution. In the case of mouse Hepa1-6 cells, no binding was observed in the control samples or at a dilution of 1/400. However, a significant increase in binding was observed with increasing antibody concentration. At a dilution of 1/200, approximately 5% of cells showed binding, which further increased to 7% at 1/100 dilution, and reached a maximum of 10% at a dilution of 1/50.

Furthermore, a comparison was made between mouse hepatocytes from wild-type (WT) and claudin-3 knockout (KO) mice. The background signal (without the antibody) was similar between WT and KO hepatocytes, accounting for approximately 2/3% of the total cells. Upon addition of the anti-claudin-3 antibody, the background signal increased to 3% in KO hepatocytes, while in WT hepatocytes, the signal increased to 10%.

Overall, these results demonstrate the specific binding of the anti-claudin-3 antibody to both human and mouse cells, with a dose-dependent increase in binding. Additionally, the comparison between WT and KO hepatocytes suggests a claudin-3-dependent binding of the UB-VS003 antibody.

### GalNAc Functionalized UB-VS003 Induce Claudin-3 Knockdown.

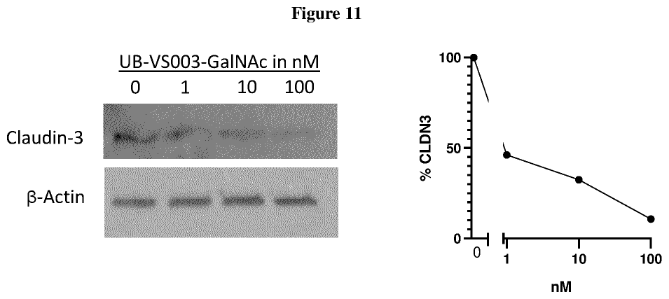

FIG. 11 displays a Western blot analysis depicting the expression levels of claudin-3 and beta-actin as a loading control. The ratio of claudin-3 to beta-actin was quantified to assess the relative abundance of claudin-3 protein.

In the control sample, the ratio of claudin-3 to beta-actin was set at 100%, indicating that claudin-3 was fully expressed under normal conditions. Following treatment with the GalNAc-anti claudin-3 antibody for 24 hours, a dose-dependent decrease in the claudin-3/beta-actin ratio was observed, reflecting a reduction in claudin-3 protein levels.

At a concentration of 1 nM of the GalNAc-anti claudin-3 antibody, the claudin-3/beta-actin ratio decreased to approximately 45% compared to the control sample. This reduction became more pronounced as the antibody concentration increased further. At 30 nM, the ratio dropped to around 30%, and at the highest concentration tested, 100 nM, the ratio decreased to approximately 10%.

These findings demonstrate that the addition of the GalNAc-anti claudin-3 antibody led to a dose-dependent decrease in the expression levels of claudin-3 protein. It suggests that the functionalized antibody efficiently triggered the lysosomal degradation of claudin-3 in human hepatocytes.

Taken together, human claudin-3 GalNAc siRNA's, as well as the human claudin-3 GalNAc antibody UB-VS003, could be efficiently used to deplete human claudin-3. Both modalities will now be used further in pre-clinical and clinical studies, with the aim to treat cholestasis and/or fibrosis associated with cholestasis.

### REFERENCES

1. EASL Hepahealth report. Risk Factors and the Burden of Liver Disease in Europe and Selected Central Asian Countries. Epub ahead of print 2018. DOI: https://ilc-congress.eu/wp-content/uploads/2018/hepahealth/EASL-HEPAHEALTH-Report.pdf.
2. Santiago P, Scheinberg A R, Levy C. Cholestatic liver diseases: new targets, new therapies. Ther Adv Gastroenterol 2018; 11:1756284818787400.
3. Hirschfield G M, Gershwin M E, Strauss R, Mayo M J, Levy C, Zou B, Johanns J, Nnane I P, Dasgupta B, Li K, Selmi C, Marschall H-U, Jones D, Lindor K, PURIFI Study Group. Ustekinumab for patients with primary biliary cholangitis who have an inadequate response to ursodeoxycholic acid: A proof-of-concept study. Hepatol Baltim Md 2016; 64:189-199.
4. Myers R P, Swain M G, Lee S S, Shaheen AAM, Burak K W. B-cell depletion with rituximab in patients with primary biliary cirrhosis refractory to ursodeoxycholic acid. Am J Gastroenterol 2013; 108:933-941.
5. Tsuda M, Moritoki Y, Lian Z-X, Zhang W, Yoshida K, Wakabayashi K, Yang G-X, Nakatani T, Vierling J, Lindor K, Gershwin M E, Bowlus C L. Biochemical and immunologic effects of rituximab in patients with primary biliary cirrhosis and an incomplete response to ursodeoxycholic acid. Hepatol Baltim Md 2012; 55:512-521.
6. Tabibian J H, Gossard A, El-Youssef M, Eaton J E, Petz J, Jorgensen R, Enders F B, Tabibian A, Lindor K D. Prospective Clinical Trial of Rifaximin Therapy for Patients With Primary Sclerosing Cholangitis. Am J Ther 2017; 24: e56-e63.
7. Rahimpour S, Nasiri-Toosi M, Khalili H, Ebrahimi-Daryani N, Nouri-Taromlou M K, Azizi Z. A Triple Blinded, Randomized, Placebo-Controlled Clinical Trial to Evaluate the Efficacy and Safety of Oral Vancomycin in Primary Sclerosing Cholangitis: a Pilot Study. J Gastrointest Liver Dis JGLD 2016; 25:457-464.
8. Mayo M J, Wigg A J, Leggett B A, Arnold H, Thompson A J, Weltman M, Carey E J, Muir A J, Ling L, Rossi S J, DePaoli A M. NGM282 for Treatment of Patients With Primary Biliary Cholangitis: A Multicenter, Randomized, Double-Blind, Placebo-Controlled Trial. Hepatol Commun 2018; 2:1037-1050.
9. Comeglio P, Filippi S, Sarchielli E, Morelli A, Cellai I, Corcetto F, Corno C, Maneschi E, Pini A, Adorini L, Vannelli G B, Maggi M, Vignozzi L. Anti-fibrotic effects of chronic treatment with the selective FXR agonist obeticholic acid in the bleomycin-induced rat model of pulmonary fibrosis. J Steroid Biochem Mol Biol 2017; 168:26-37.
10. Nevens F, Andreone P, Mazzella G, Strasser S I, Bowlus C, Invernizzi P, Drenth J P H, Pockros P J, Regula J, Beuers U, Trauner M, Jones D E, Floreani A, Hohenester S, Luketic V, Shiffman M, van Erpecum K J, Vargas V, Vincent C, Hirschfield G M, Shah H, Hansen B, Lindor K D, Marschall H-U, Kowdley K V, Hooshmand-Rad R, Marmon T, Sheeron S, Pencek R, MacConell L, Pruzanski M, Shapiro D. A Placebo-Controlled Trial of Obeticholic Acid in Primary Biliary Cholangitis. N Engl J Med 2016; 375:631-643.
11. Levy C, Peter J A, Nelson D R, Keach J, Petz J, Cabrera R, Clark V, Firpi R J, Morelli G, Soldevila-Pico C, Lindor K. Pilot study: fenofibrate for patients with primary biliary cirrhosis and an incomplete response to ursodeoxycholic acid: Pilot study: fenofibrate with UDCA for primary biliary cirrhosis. Aliment Pharmacol Ther 2011; 33:235-242.
12. Jones D, Boudes P F, Swain M G, Bowlus C L, Galambos M R, Bacon B R, Doerffel Y, Gitlin N, Gordon S C, Odin J A, Sheridan D, Wörns M-A, Clark V, Corless L, Hartmann H, Jonas M E, Kremer A E, Mells G F, Buggisch P, Freilich B L, Levy C, Vierling J M, Bernstein D E, Hartleb M, Janczewska E, Rochling F, Shah H, Shiffman M L, Smith J H, Choi Y-J, Steinberg A, Varga M, Chera H, Martin R, McWherter C A, Hirschfield G M. Seladelpar (MBX-8025), a selective PPAR-8 agonist, in patients with primary biliary cholangitis with an inadequate response to ursodeoxycholic acid: a double-blind, randomised, placebo-controlled, phase 2, proof-of-concept study. Lancet Gastroenterol Hepatol 2017; 2:716-726.
13. Corpechot C, Chazouillères O, Rousseau A, Le Gruyer A, Habersetzer F, Mathurin P, Goria O, Potier P, Minello A, Silvain C, Abergel A, Debette-Gratien M, Larrey D, Roux O, Bronowicki J-P, Boursier J, de Ledinghen V, Heurgue-Berlot A, Nguyen-Khac E, Zoulim F, Ollivier-Hourmand I, Zarski J-P, Nkontchou G, Lemoinne S, Humbert L, Rainteau D, Lefèvre G, de Chaisemartin L, Chollet-Martin S, Gaouar F, Admane F-H, Simon T, Poupon R. A Placebo-Controlled Trial of Bezafibrate in Primary Biliary Cholangitis. N Engl J Med 2018; 378:2171-2181.
14. Merlen G, Kahale N, Ursic-Bedoya J, Bidault-Jourdainne V, Simerabet H, Doignon I, Tanfin Z, Garcin I, Péan N, Gautherot J, Davit-Spraul A, Guettier C, Humbert L, Rainteau D, Ebnet K, Ullmer C, Cassio D, Tordjmann T. TGR5-dependent hepatoprotection through the regulation of biliary epithelium barrier function. Gut 2020; 69:146-157.
15. Georgiev P, Navarini A A, Eloranta J J, Lang K S, Kullak-Ublick G A, Nocito A, Dahm F, Jochum W, Graf R, Clavien P-A. Cholestasis protects the liver from ischaemic injury and post-ischaemic inflammation in the mouse. Gut 2007; 56:121-128.
16. Moghadamrad S, McCoy K D, Geuking M B, Sägesser H, Kirundi J, Macpherson A J, De Gottardi A. Attenuated portal hypertension in germ-free mice: Function of bacterial flora on the development of mesenteric lymphatic and blood vessels: HEPATOLOGY, Vol. XX, NO. X, 2015. Hepatology 2015; 61:1685-1695.
17. Gómez C, Stücheli S, Kratschmar D V, Bouitbir J, Odermatt A. Development and Validation of a Highly Sensitive LC-MS/MS Method for the Analysis of Bile Acids in Serum, Plasma, and Liver Tissue Samples. Metabolites; 10. Epub ahead of print Jul. 9, 2020. DOI: 10.3390/metabo10070282.
18. Penno C A, Arsenijevic D, Da Cunha T, Kullak-Ublick G A, Montani J-P, Odermatt A. Quantification of multiple bile acids in uninephrectomized rats using ultra-performance liquid chromatography-tandem mass spectrometry. Anal Methods 2013; 5:1155.
19. Joshi N, Ray J L, Kopec A K, Luyendyk J P. Dose-dependent effects of alpha-naphthylisothiocyanate disconnect biliary fibrosis from hepatocellular necrosis. J Biochem Mol Toxicol 2017; 31:1-7.
20. Chang M-L. Comparison of murine cirrhosis models induced by hepatotoxin administration and common bile duct ligation. World J Gastroenterol 2005; 11:4167.
21. Chihara M, Ikebuchi R, Otsuka S, Ichii O, Hashimoto Y, Suzuki A, Saga Y, Kon Y. Mice Stage-Specific Claudin 3 Expression Regulates Progression of Meiosis in Early Stage Spermatocytes1. Biol Reprod; 89. Epub ahead of print Jul. 1, 2013. DOI: 10.1095/biolreprod. 113.107847.
22. Zhang T, Yu F, Guo L, Chen M, Yuan X, Wu B. Small Heterodimer Partner Regulates Circadian Cytochromes p450 and Drug-Induced Hepatotoxicity. Theranostics 2018; 8:5246-5258.
23. Di-Luoffo M, Brousseau C, Bergeron F, Tremblay J J. The Transcription Factor MEF2 Is a Novel Regulator of Gsta Gene Class in Mouse MA-10 Leydig Cells. Endocrinology 2015; 156:4695-4706.
24. Ramos Pittol J M, Milona A, Morris I, Willemsen E C L, van der Veen S W, Kalkhoven E, van Mil S W C. FXR Isoforms Control Different Metabolic Functions in Liver Cells via Binding to Specific DNA Motifs. Gastroenterology 2020; 159:1853-1865.e10.
25. Merrell M D, Nyagode B A, Clarke J D, Cherrington N J, Morgan E T. Selective and Cytokine-Dependent Regulation of Hepatic Transporters and Bile Acid Homeostasis during Infectious Colitis in Mice. Drug Metab Dispos 2014; 42:596-602.
26. Liu J, He Y-Y, Chignell C F, Clark J, Myers P, Saavedra J E, Waalkes M P. Limited protective role of V-PYRRO/NO against cholestasis produced by alpha-naphthylisothiocyanate in mice. Biochem Pharmacol 2005; 70:144-151.
27. Baier F A, Sánchez-Taltavull D, Yarahmadov T, Castellà C G, Jebbawi F, Keogh A, Tombolini R, Odriozola A, Dias M C, Deutsch U, Furuse M, Engelhardt B, Zuber B, Odermatt A, Candinas D, Stroka D. Loss of Claudin-3 Impairs Hepatic Metabolism, Biliary Barrier Function, and Cell Proliferation in the Murine Liver. Cell Mol Gastroenterol Hepatol 2021; S2352345X21000746.
28. Croce A C, Ferrigno A, Santin G, Piccolini V M, Bottiroli G, Vairetti M. Autofluorescence of liver tissue and bile: Organ functionality monitoring during ischemia and reoxygenation: AUTOFLUORESCENCE OF LIVER TISSUE AND BILE. Lasers Surg Med 2014; 46:412-421.
29. Cai S-Y, Boyer J L. The role of bile acids in cholestatic liver injury. Ann Transl Med 2021; 9:737-737.
30. Tanaka H, Imasato M, Yamazaki Y, Matsumoto K, Kunimoto K, Delpierre J, Meyer K, Zerial M, Kitamura N, Watanabe M, Tamura A, Tsukita S. Claudin-3 regulates bile canalicular paracellular barrier and cholesterol gallstone core formation in mice. J Hepatol 2018; 69:1308-1316.
31. Yamaga K, Murota H, Tamura A, Miyata H, Ohmi M, Kikuta J, Ishii M, Tsukita S, Katayama I. Claudin-3 Loss Causes Leakage of Sweat from the Sweat Gland to Contribute to the Pathogenesis of Atopic Dermatitis. J Invest Dermatol 2018; 138:1279-1287.
32. Fickert P, Pollheimer M J, Österreicher C H, Trauner M. Animal Models of Cholestasis. In: Animal Models for the Study of Human Disease. Elsevier; pp. 331-349.
33. Springer A D, Dowdy S F. GalNAc-siRNA Conjugates: Leading the Way for Delivery of RNAi Therapeutics. Nucleic Acid Ther 2018; 28:109-118.

1. An agent which inhibits the expression and/or activity of Claudin-3 for use in a method of prevention, delay of progression or treatment of cholestasis and/or fibrosis associated with cholestasis.

2. The agent for use according to claim 1, wherein the agent which inhibits the expression and/or activity of Claudin-3 is a siRNA targeting Claudin-3 or an antibody or a fragment thereof which binds to Claudin-3.

3. The agent for use according to claim 1, wherein the agent inhibits the expression of Claudin-3.

4. The agent for use according to claim 3, wherein the agent which inhibits the expression of Claudin-3 is a nucleic acid targeting a gene or mRNA coding for Claudin-3.

5. The agent for use according to claim 3, wherein the agent which inhibits the expression of Claudin-3 is a siRNA targeting Claudin-3.

6. The agent for use according to claim 2 or 5, wherein the siRNA targeting Claudin-3 is conjugated to a compound which facilitates delivery of the siRNA to the liver.

7. The agent for use according to claim 2 or 5, wherein the siRNA targeting Claudin-3 is a N-acetylgalactosamine (GalNAc) siRNA conjugate.

8. The agent for use according to any one of claims 5-7, wherein the siRNA is selected from the group consisting of siRNA comprising the sequence as shown in any of SEQ ID NOs: 1, 2, 5, 6 or 33-168 or siRNA which is at least 95% identical, more preferably 96%, 97%, 98%, 99% or 100% identical to the siRNA comprising the sequence as shown in any of SEQ ID NO: 1, 2, 5, 6 or 33-168.

9. The agent for use according to any one of claims 5-7, wherein the siRNA is selected from the group consisting of siRNA comprising the sequence as shown in any of SEQ ID NOs: 33-168 or siRNA which is at least 95% identical, more preferably 96%, 97%, 98%, 99% or 100% identical to the siRNA comprising the sequence as shown in any of SEQ ID NO: 33-168.

10. The agent for use according to any one of claims 5-7, wherein the siRNA is selected from the group consisting of siRNA comprising the sequence as shown in any of SEQ ID NOs: 33-100 or siRNA which is at least 95% identical, more preferably 96%, 97%, 98%, 99% or 100% identical to the siRNA comprising the sequence as shown in any of SEQ ID NO: 33-100.

11. The agent for use according to any one of claims 5-7, wherein the siRNA is selected from the group consisting of siRNA comprising the sequence as shown in SEQ ID NO:1, siRNA comprising the sequence as shown in SEQ ID NO: 2, siRNA which is at least 95% identical, more preferably 96%, 97%, 98%, 99% or 100% identical to the siRNA comprising the sequence as shown in SEQ ID NO:1 and siRNA which is at least 95% identical, more preferably 96%, 97%, 98%, 99% or 100% identical to the siRNA comprising the sequence as shown in SEQ ID NO: 2.

12. The agent for use according to claim 1, wherein the agent inhibits the activity of Claudin-3.

13. The agent for use according to claim 12, wherein the agent which inhibits the activity of Claudin-3 is selected from the group consisting of an antibody or a fragment thereof which binds to Claudin-3 and a toxin which binds to Claudin-3

14. The agent for use according to claim 13, wherein the toxin which binds to Claudin-3 is the Clostridium perfringens enterotoxin (CPE) or a fragment or variant thereof.

15. The agent for use according to claim 12, wherein the agent which inhibits the activity of Claudin-3 is an antibody or a fragment thereof which binds to Claudin-3.

16. The agent for use according to claim 15, wherein the antibody or a fragment thereof which binds to Claudin-3 comprises a light chain comprising the amino acid sequence as shown in SEQ ID NO: 169 and/or a heavy chain comprising the amino acid sequence as shown in SEQ ID NO: 170.

17. The agent for use according to claim 15 or 16, wherein the antibody or a fragment thereof which binds to Claudin-3 is conjugated to a compound which facilitates delivery of the antibody to the liver.

18. The agent for use according to claim 15 or 16, wherein the antibody or a fragment thereof which binds to Claudin-3 is a N-acetylgalactosamine (GalNAc) antibody conjugate.

19. The agent for use according to any one of claims 1-18, wherein cholestasis is a disease selected from the group consisting of obstructive cholestasis, nonobstructive cholestasis, bile duct diseases and defects in biliary function.

20. A composition comprising an agent according to any one of claims 1-18 and optionally a pharmaceutically acceptable carrier for use in a method of prevention, delay of progression or treatment of cholestasis and/or fibrosis associated with cholestasis.

21. A dosage form for the prevention, delay of progression or treatment of cholestasis and/or fibrosis associated with cholestasis, comprising an agent according to any one of claims 1-18 or a composition comprising said agent according to claim 20, and optionally a pharmaceutically acceptable carrier.

22. A siRNA targeting Claudin-3.

23. A siRNA targeting Claudin-3 selected from the group consisting of siRNA comprising the sequence as shown in any of SEQ ID NOs: 1, 2, 5, 6 or 33-168 or siRNA which is at least 95% identical, more preferably 96%, 97%, 98%, 99% or 100% identical to the siRNA comprising the sequence as shown in any of SEQ ID NO: 1, 2, 5, 6 or 33-168.

24. A siRNA targeting Claudin-3 selected from the group consisting of siRNA comprising the sequence as shown in any of SEQ ID NOs: 33-168 or siRNA which is at least 95% identical, more preferably 96%, 97%, 98%, 99% or 100% identical to the siRNA comprising the sequence as shown in any of SEQ ID NO: 33-168.

25. A siRNA targeting Claudin-3 selected from the group consisting of siRNA comprising the sequence as shown in any of SEQ ID NOs: 33-100 or siRNA which is at least 95% identical, more preferably 96%, 97%, 98%, 99% or 100% identical to the siRNA comprising the sequence as shown in any of SEQ ID NO: 33-100.

26. A siRNA targeting Claudin-3 selected from the group consisting of siRNA comprising the sequence as shown in SEQ ID NO:1, siRNA comprising the sequence as shown in SEQ ID NO: 2, siRNA which is at least 95% identical, more preferably 96%, 97%, 98%, 99% or 100% identical to the siRNA comprising the sequence as shown in SEQ ID NO:1 and siRNA which is at least 95% identical, more preferably 96%, 97%, 98%, 99% or 100% identical to the siRNA comprising the sequence as shown in SEQ ID NO: 2.

27. An antibody or a fragment thereof which binds to Claudin-3.

28. The antibody or a fragment thereof according to claim 27, wherein the antibody or a fragment thereof which binds to Claudin-3 is a human antibody.

29. The antibody or a fragment thereof according to claim 27 or 28, wherein the antibody or a fragment thereof which binds to Claudin-3 comprises a light chain comprising the amino acid sequence as shown in SEQ ID NO: 169 and/or a heavy chain comprising the amino acid sequence as shown in SEQ ID NO: 170.

30. The antibody or a fragment thereof to any one of claims 27-29, wherein the antibody or a fragment thereof which binds to Claudin-3 is conjugated to a compound which facilitates delivery of the antibody to the liver.

31. The antibody or a fragment thereof according to any one of claims 27-29, wherein the antibody or a fragment thereof which binds to Claudin-3 is a N-acetylgalactosamine (GalNAc) antibody conjugate.
